(19) United States
(12) Patent Application Publication
(10) Pub. No.: US 2023/0065143 A1
(43) Pub. Date: Mar. 2, 2023

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE AND DISPLAY DEVICE

(71) Applicant: SAMSUNG SDI CO., LTD., Yongin-si (KR)

(72) Inventors: Mijin LEE, Suwon-si (KR); Ho Kuk JUNG, Suwon-si (KR); Youngkyoung JO, Suwon-si (KR); Jonghoon KIM, Suwon-si (KR); Jinhyun LUI, Suwon-si (KR); Jongwoo WON, Suwon-si (KR); Youngmook LIM, Suwon-si (KR); Hyungyu LEE, Suwon-si (KR); Sung-Hyun JUNG, Suwon-si (KR)

(21) Appl. No.: 17/824,175

(22) Filed: May 25, 2022

(30) Foreign Application Priority Data
Jul. 6, 2021 (KR) 10-2021-0088621
May 16, 2022 (KR) 10-2022-0059689

(51) Int. Cl. C07D 209/82; C07D 405/14; C07D 405/04; C07D 493/04; C07D 409/14; C07D 409/10; C07D 405/10; C07D 495/04; C07D 491/048; C07D 403/10; C07D 403/14; C07D 403/04; C07D 487/04; C07D 307/91; C07D 333/76; C07F 15/00; C07C 211/54; H01L 51/00

(52) U.S. Cl. CPC C07D 209/82; C07D 405/14; C07D 405/04; C07D 493/04; C07D 409/14; C07D 409/10; C07D 405/10; C07D 495/04; C07D 491/048; C07D 403/10; C07D 403/14; C07D 403/04; C07D 487/04; C07D 307/91; C07D 333/76; C07F 15/0086; C07F 15/0033; C07C 211/54; H01L 51/0087; H01L 51/0085; H01L 51/0074; H01L 51/0073; H01L 51/0072; H01L 51/0067; H01L 51/0054; H01L 51/0056; H01L 51/0071; H01L 51/0059; C07B 2200/05; H01L 51/5012

(57) ABSTRACT

A compound for an organic optoelectronic device, a composition for an organic optoelectronic device including the same, an organic optoelectronic device, and a display device, the compound being represented by Chemical Formula 1:

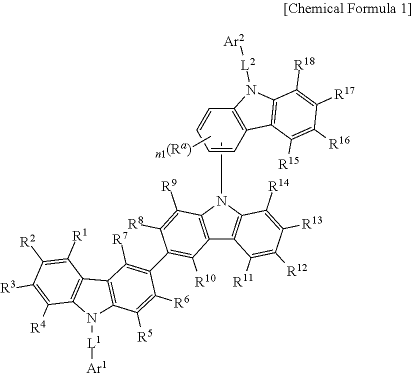

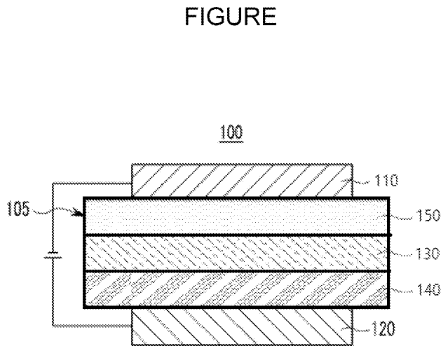

### CROSS-REFERENCE TO RELATED APPLICATION

This application claims priority to and the benefit of Korean Patent Application No. 10-2021-0088621 filed in the Korean Intellectual Property Office on Jul. 6, 2021, and Korean Patent Application No. 10-2022-0059689 filed in the Korean Intellectual Property Office on May 16, 2022, the entire contents of which are incorporated herein by reference.

### BACKGROUND

### 1. Field

Embodiments relate to a compound for an organic optoelectronic device, a composition for an organic optoelectronic device, an organic optoelectronic device, and a display device.

### 2. Description of the Related Art

An organic optoelectronic device (e.g., an organic optoelectronic diode) is a device capable of converting electrical energy and optical energy to each other.

An organic optoelectronic device may be classified as follows in accordance with its driving principles. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device may include an organic photoelectric element, an organic light emitting diode, an organic solar cell, and an organic photo conductor drum.

Of these, an organic light emitting diode (OLED) has recently drawn attention due to an increase in demand for flat panel displays. The organic light emitting diode is a device that converts electrical energy into light, and the performance of the organic light emitting diode is greatly influenced by an organic material between electrodes.

### SUMMARY

The embodiments may be realized by providing a compound for an organic optoelectronic device, the compound being represented by Chemical Formula 1:

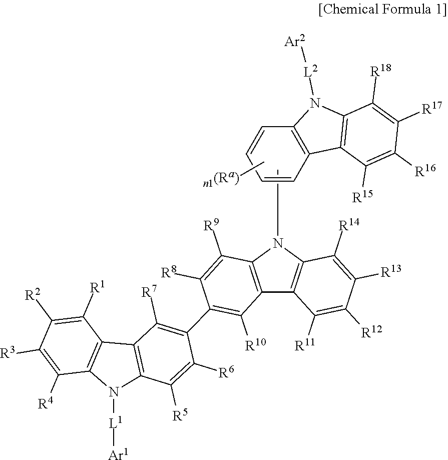

wherein, in Chemical Formula 1, R^{a} and R¹ to R¹⁸ are each independently hydrogen, deuterium, a halogen, a substituted or unsubstituted C1 to C30 alkyl group, or a substituted or unsubstituted C6 to C30 aryl group, n1 is an integer of 1 to 3, Ar¹ and Ar² are each independently a substituted or unsubstituted C1 to C30 alkyl group, a substituted or unsubstituted C6 to C30 aryl group, or a substituted or unsubstituted C2 to C30 heterocyclic group, and L¹ and L² are each independently a single bond, a substituted or unsubstituted C6 to C30 arylene group, or a substituted or unsubstituted C2 to C30 heterocyclic group, provided that said compound meets one or more of the following:

at least one of R^{a} and R¹ to R¹⁸ is deuterium;

at least one of R^{a} and R¹ to R¹⁸ is a C1 to C30 alkyl group substituted with at least one deuterium or a C6 to C30 aryl group substituted with at least one deuterium; or

at least one of Ar¹ and Ar² is a C1 to C30 alkyl group substituted with at least one deuterium, a C6 to C30 aryl group substituted with at least one deuterium, or a C2 to C30 heterocyclic group substituted with at least one deuterium.

The embodiments may be realized by providing a composition for an organic optoelectronic device, the composition including a first compound; and a second compound, wherein the first compound is the compound for an organic optoelectronic device according to an embodiment, and the second compound is a compound represented by Chemical Formula 2:

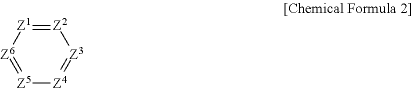

in Chemical Formula 2, Z¹ is N or C-L³-R¹⁹, Z² is N or C-L⁴-R²⁰, Z³ is N or C-L⁵-R²¹, Z⁴ is N or C-L⁶-R²², Z⁵ is N or C-L⁷-R²³, Z⁶ is N or C-L⁸-R²⁴, provided that at least two of Z¹ to Z⁶ are N, L³ to L⁸ are each independently a single bond, a substituted or unsubstituted C6 to C20 arylene group, a substituted or unsubstituted C2 to C20 heterocyclic group, or a combination thereof, R¹⁹ to R²⁴ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof, R¹⁹ to R²⁴ are each separately present or adjacent ones thereof are linked to each other to provide a substituted or unsubstituted aliphatic monocyclic ring, a substituted or unsubstituted aliphatic polycyclic ring, a substituted or unsubstituted aromatic monocyclic ring, a substituted or unsubstituted to form an aromatic polycyclic ring, a substituted or unsubstituted heteroaromatic monocyclic ring, or a substituted or unsubstituted heteroaromatic polycyclic ring, and when R¹⁹ to R²⁴ are each separately present, at least one of R¹⁹ to R²⁴ is a substituted or unsubstituted C10 to C30 aryl group or a substituted or unsubstituted C2 to C30 heterocyclic group.

The embodiments may be realized by providing an organic optoelectronic device including an anode and a cathode facing each other, at least one organic layer between the anode and the cathode, wherein the at least one organic layer includes the compound for an organic optoelectronic device according to an embodiment.

The embodiments may be realized by providing an organic optoelectronic device including an anode and a cathode facing each other, at least one organic layer between the anode and the cathode, wherein the at least one organic layer includes the composition for an organic optoelectronic device according to an embodiment.

The embodiments may be realized by providing a display device including the organic optoelectronic device according to an embodiment.

### BRIEF DESCRIPTION OF THE DRAWING

Features will be apparent to those of skill in the art by describing in detail exemplary embodiments with reference to the attached drawing in which:

the FIGURE is a cross-sectional view illustrating an organic light emitting diode according to an embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. It will also be understood that when a layer or element is referred to as being “on” another layer or element, it can be directly on the other layer or element, or intervening layers may also be present. In addition, it will also be understood that when a layer is referred to as being “between” two layers, it can be the only layer between the two layers, or one or more intervening layers may also be present. Like reference numerals refer to like elements throughout.

In one example of the present disclosure, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In specific example of the present disclosure, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In specific example of the present disclosure, the “substituted” refers to replacement of at least one hydrogen of a substituent or a compound by deuterium, a C1 to C20 alkyl group, a C6 to C30 aryl group, or a cyano group. In specific example of the present disclosure, the “substituted” refers to replacement of at least one hydrogen of a substituent or a compound by deuterium, a C1 to C5 alkyl group, a C6 to C18 aryl group, or a cyano group. In specific example of the present disclosure, the “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen substitution (—H)” may include deuterium substitution (-D) or “tritium substitution (-T).

As used herein, when a definition is not otherwise provided, “hetero” refers to one including one to three heteroatoms selected from N, O, S, P, and Si, and remaining carbons in one functional group.

As used herein, “an aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

As used herein, “a heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “a heteroaryl group” may refer to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, a substituted or unsubstituted furanyl group, or a combination thereof, but is not limited thereto.

More specifically, the substituted or unsubstituted C2 to C30 heterocyclic group may be a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a compound for an organic optoelectronic device according to an embodiment is described.

A compound for an organic optoelectronic device according to an embodiment may be represented by, e.g., Chemical Formula 1.

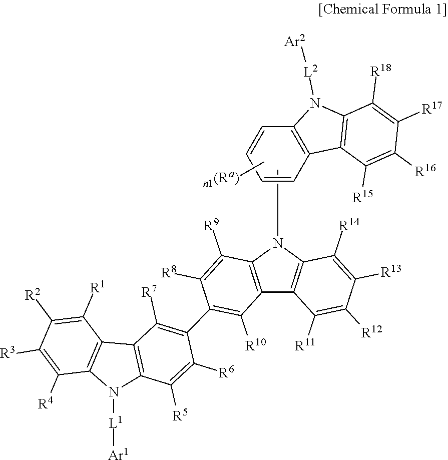

In Chemical Formula 1, R^{a} and R¹ to R¹⁸ may each independently be or include, e.g., hydrogen, deuterium, a halogen, a substituted or unsubstituted C1 to C30 alkyl group, or a substituted or unsubstituted C6 to C30 aryl group. As used herein, the term “or” is not an exclusive term, e.g., “A or B” would include A, B, or A and B.

n1 may be, e.g., an integer of, e.g., 1 to 3.

Ar¹ and Ar² may each independently be or include, e.g., a substituted or unsubstituted C1 to C30 alkyl group, a substituted or unsubstituted C6 to C30 aryl group, or a substituted or unsubstituted C2 to C30 heterocyclic group.

L¹ and L² may each independently be or include, e.g., a single bond, a substituted or unsubstituted C6 to C30 arylene group, or a substituted or unsubstituted C2 to C30 heterocyclic group.

In an implementation, one or more of the following conditions of Chemical Formula 1 may be met:

at least one of R^{a} and R¹ to R¹⁸ may be deuterium;

at least one of R^{a} and R¹ to R¹⁸ may be a C1 to C30 alkyl group substituted with at least one deuterium, or a C6 to C30 aryl group substituted with at least one deuterium;

or

at least one of Ar¹ and Ar² may be a C1 to C30 alkyl group substituted with at least one deuterium, a C6 to C30 aryl group substituted with at least one deuterium, or a C2 to C30 heterocyclic group substituted with at least one deuterium.

The compound substituted with deuterium, compared with a compound only including hydrogen, may have lower ground state energy due to lower zero-point energy and lower vibration energy and reduced intermolecular interaction and thus make a thin film into an amorphous state, further improving heat resistance and effectively improving a life-span of an organic light emitting diode manufactured by applying the same. Accordingly, a driving voltage of the organic light emitting diode may be effectively lowered by greatly improving the life-span through the deuterium substitution and also, improving hole injection and transport characteristics through a structure in which carbazole is directly bonded on a nitrogen atom of a biscarbazole moiety.

In an implementation, Chemical Formula 1 may be represented by, e.g., one of Chemical Formula 1-I to Chemical Formula 1-IV.

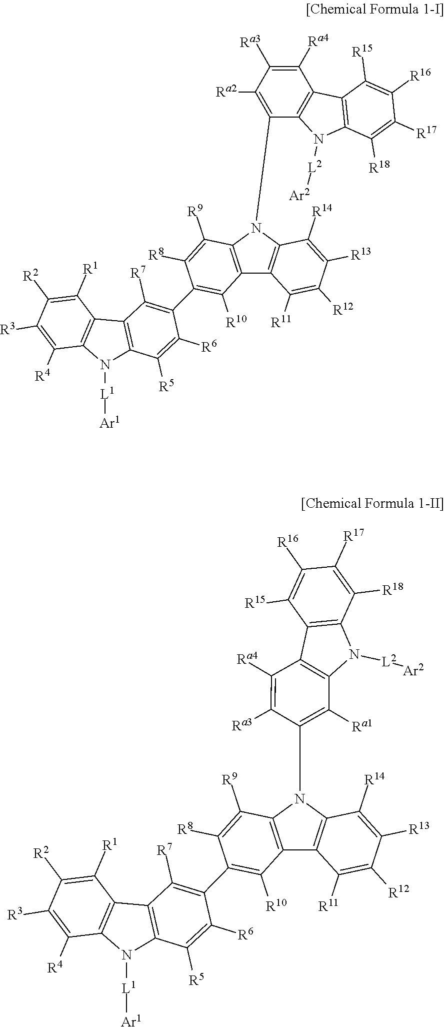

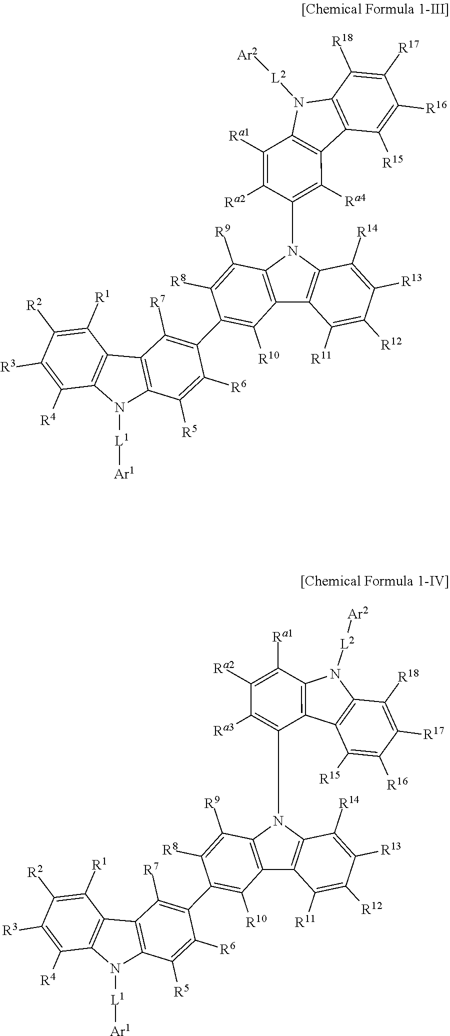

In Chemical Formula 1-I to Chemical Formula 1-IV, R¹ to R¹⁸, Ar¹, Ar², L¹ and L² may be defined the same as those described above, and R^{a1} to R^{a4} may each independently be defined the same as R^{a}.

In an implementation, Chemical Formula 1 may be represented by, e.g., Chemical Formula 1-II or Chemical Formula 1-III.

In an implementation, Chemical Formula 1 may be represented by, e.g., Chemical Formula 1A.

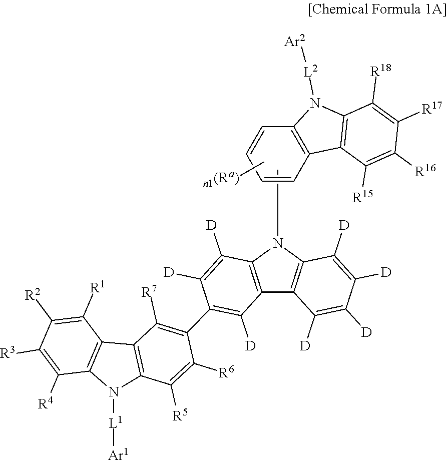

In Chemical Formula 1A, R^{a}, R¹ to R⁷, and R¹⁸ to R¹⁸ may each independently be, e.g., hydrogen, deuterium, a halogen, a substituted or unsubstituted C1 to C30 alkyl group, or a substituted or unsubstituted C6 to C30 aryl group.

n1 may be an integer of, e.g., 1 to 3.

Ar¹ and Ar² may each independently be, e.g., a substituted or unsubstituted C1 to C30 alkyl group or a substituted or unsubstituted C6 to C30 aryl group.

L¹ and L² may each independently be, e.g., a single bond or a substituted or unsubstituted C6 to C30 arylene group.

In an implementation, Chemical Formula 1 may be represented by, e.g., Chemical Formula 1B.

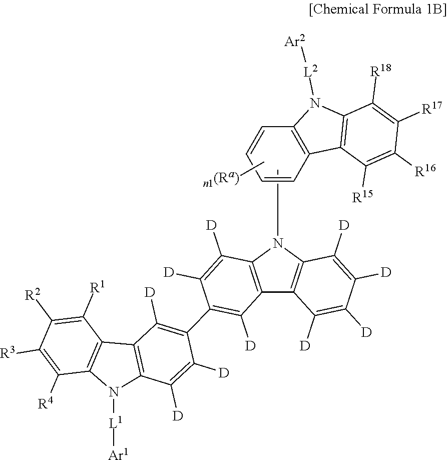

In Chemical Formula 1B, R^{a}, R¹ to R⁴, and R¹⁵ to R¹⁸ may each independently be, e.g., hydrogen, deuterium, a halogen, a substituted or unsubstituted C1 to C30 alkyl group, or a substituted or unsubstituted C6 to C30 aryl group.

n1 may be an integer of, e.g., 1 to 3.

Ar¹ and Ar² may each independently be, e.g., a substituted or unsubstituted C1 to C30 alkyl group or a substituted or unsubstituted C6 to C30 aryl group.

L¹ and L² may each independently be, e.g., a single bond or a substituted or unsubstituted C6 to C30 arylene group.

In an implementation, Chemical Formula 1 may be represented by, e.g., Chemical Formula 1C.

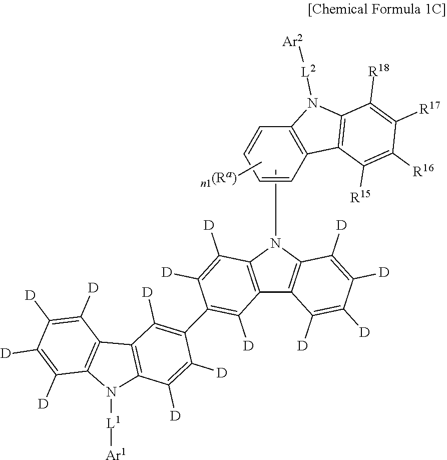

In Chemical Formula 1C, R^{a} and R¹⁵ to R¹⁸ may each independently be, e.g., hydrogen, deuterium, a halogen, a substituted or unsubstituted C1 to C30 alkyl group, or a substituted or unsubstituted C6 to C30 aryl group.

n1 may be an integer of, e.g., 1 to 3.

Ar¹ and Ar² may each independently be, e.g., a substituted or unsubstituted C1 to C30 alkyl group or a substituted or unsubstituted C6 to C30 aryl group.

L¹ and L² may each independently be, e.g., a single bond or a substituted or unsubstituted C6 to C30 arylene group.

In an implementation, at least one of R^{a} and R¹ to R¹⁸ may be deuterium; at least one of R^{a} and R¹ to R¹⁸ may be a C1 to C10 alkyl group substituted with at least one deuterium, or a C6 to C20 aryl group substituted with at least one deuterium; or at least one of Ar¹ and Ar² may be a C1 to C10 alkyl group substituted with at least one deuterium, or a C6 to C20 aryl group substituted with at least one deuterium.

In an implementation, the C1 to C10 alkyl group substituted with at least one deuterium may include, e.g., a methyl group substituted with at least one deuterium, an ethyl group substituted with at least one deuterium, an n-propyl group substituted with at least one deuterium, an iso-propyl group substituted with at least one deuterium, an n-butyl group substituted with at least one deuterium, an iso-butyl group substituted with at least one deuterium, a neo-butyl group substituted with at least one deuterium, a pentyl group substituted with at least one deuterium, a heptyl group substituted with at least one deuterium, an octyl group substituted with at least one deuterium, a nonyl group substituted with at least one deuterium, a decyl group substituted with at least one deuterium, or the like.

In an implementation, the C6 to C20 aryl group substituted with at least one deuterium may include, e.g., a phenyl group substituted with at least one deuterium, a biphenyl group substituted with at least one deuterium, a terphenyl group substituted with at least one deuterium, a naphthyl group substituted with at least one deuterium, a phenanthrenyl group substituted with at least one deuterium, an anthracenyl group substituted with at least one deuterium, a triphenylene group substituted with at least one deuterium, a fluorenyl group substituted with at least one deuterium, or the like.

In an implementation, the C6 to C20 aryl group substituted with at least one deuterium may be, e.g., a phenyl group substituted with at least one deuterium, or a biphenyl group substituted with at least one deuterium.

In an implementation, the C6 to C20 aryl group substituted with at least one deuterium may be, e.g., a group of Group I.

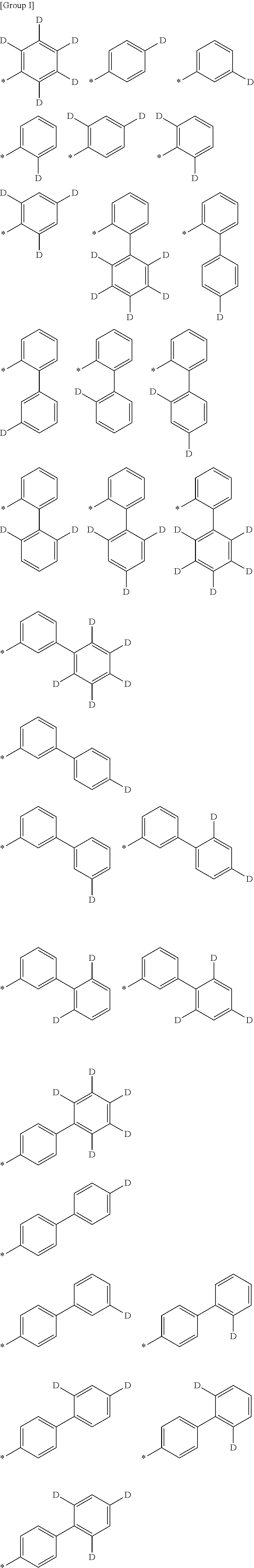

In Group I, * is a linking point.

In an implementation, the compound for an organic optoelectronic device represented by Chemical Formula 1 may include, e.g., a compound of Group 1.

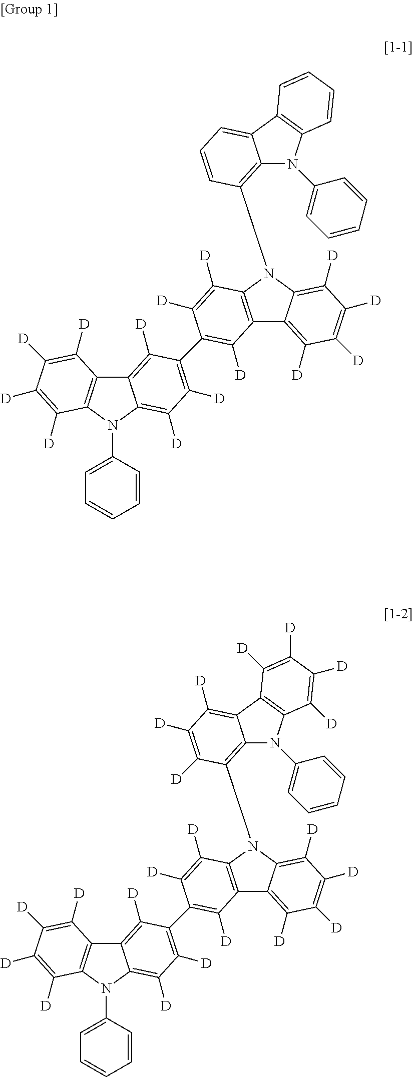 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c5ccccc5n(-c5ccccc5)c34)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c5c([2H])c([2H])c([2H])c([2H])c5n(-c5ccccc5)c34)c([2H])c([2H])c1n2-c1ccccc1]

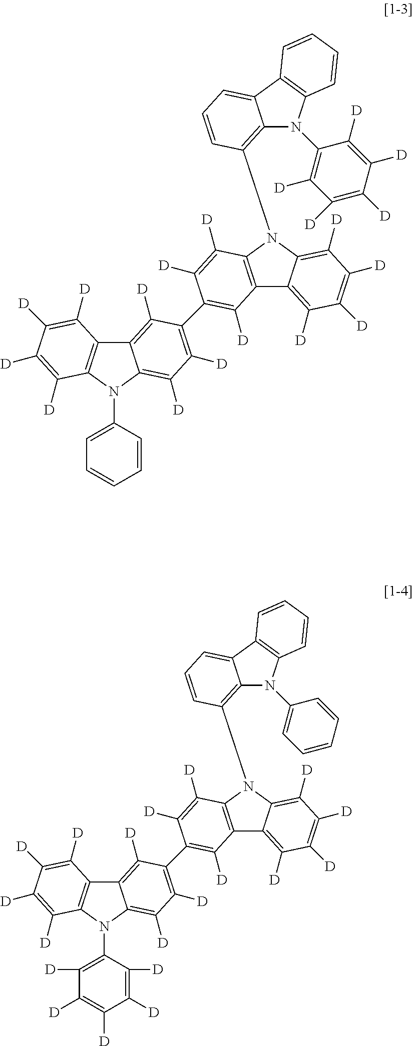 [SMILES: [2H]c1c([2H])c([2H])c(-n2c3ccccc3c3cccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c6ccccc6n(-c6ccccc6)c45)c([2H])c([2H])c32)c([2H])c1[2H]]

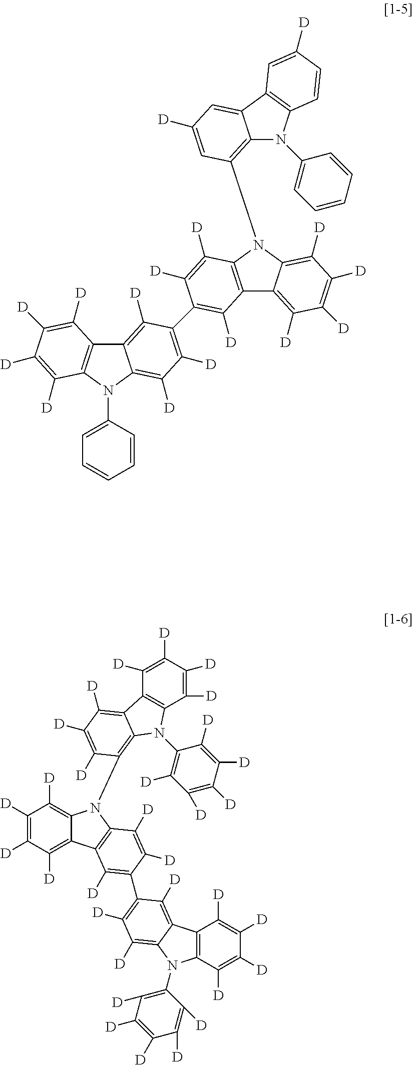 [SMILES: [2H]c1ccc2c(c1)c1cc([2H])cc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5)c([2H])c([2H])c43)c1n2-c1ccccc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c6c([2H])c([2H])c([2H])c([2H])c6n(-c6c([2H])c([2H])c([2H])c([2H])c6[2H])c45)c([2H])c([2H])c32)c([2H])c1[2H]]

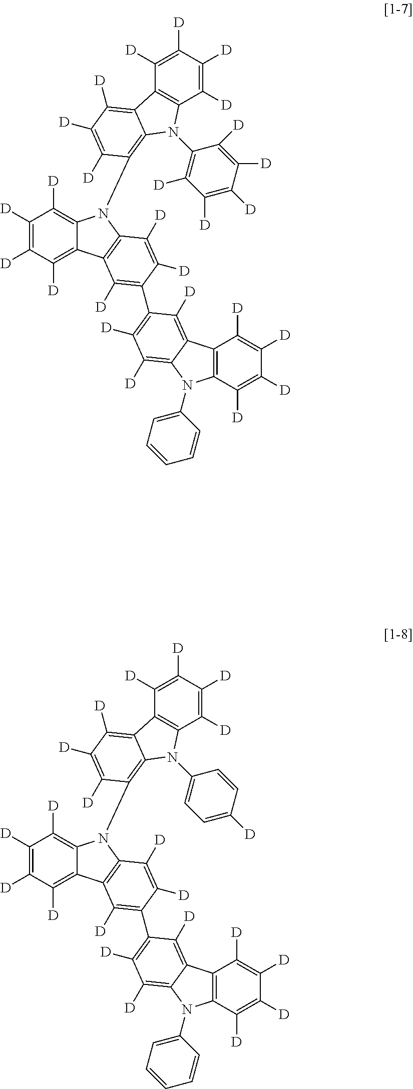 [SMILES: [2H]c1c([2H])c([2H])c(-n2c3c([2H])c([2H])c([2H])c([2H])c3c3c([2H])c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c32)c([2H])c1[2H] ; [2H]c1ccc(-n2c3c([2H])c([2H])c([2H])c([2H])c3c3c([2H])c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c32)cc1]

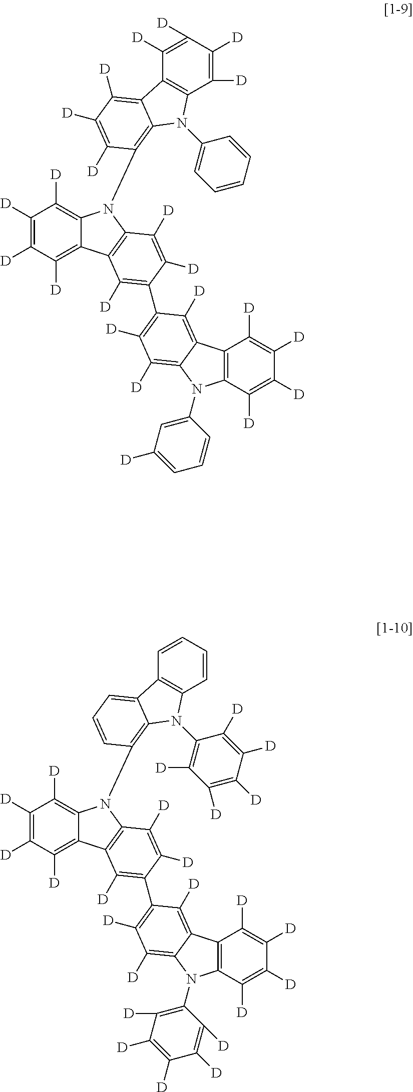 [SMILES: [2H]c1cccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c6c([2H])c([2H])c([2H])c([2H])c6n(-c6ccccc6)c45)c([2H])c([2H])c32)c1 ; [2H]c1c([2H])c([2H])c(-n2c3ccccc3c3cccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c32)c([2H])c1[2H]]

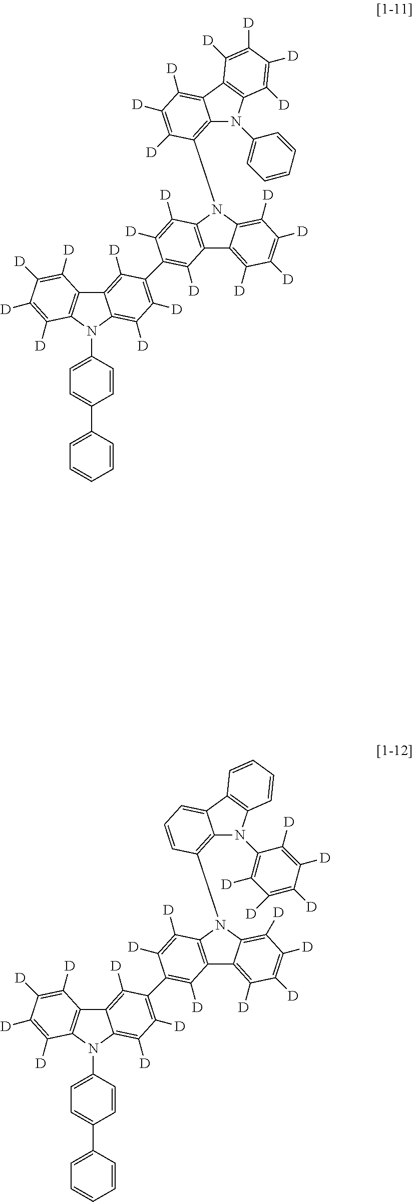 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c5c([2H])c([2H])c([2H])c([2H])c5n(-c5ccccc5)c34)c([2H])c([2H])c1n2-c1ccc(-c2ccccc2)cc1 ; [2H]c1c([2H])c([2H])c(-n2c3ccccc3c3cccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccc(-c7ccccc7)cc6)c([2H])c([2H])c54)c32)c([2H])c1[2H]]

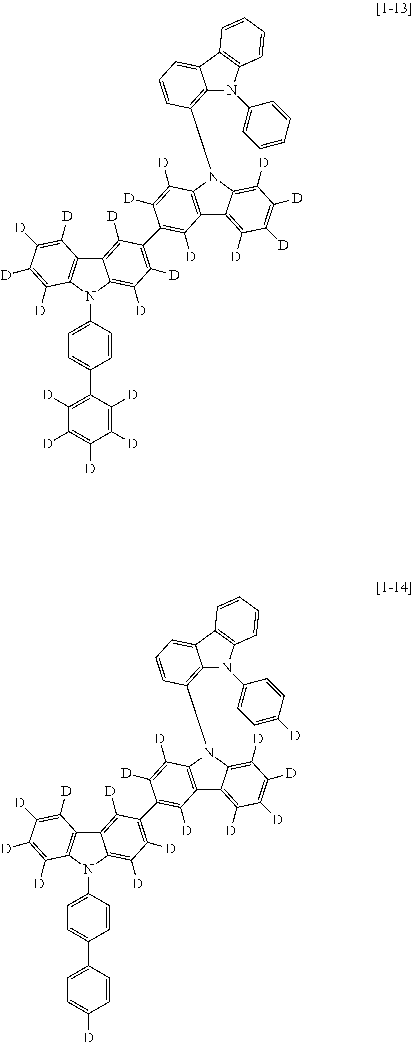 [SMILES: [2H]c1c([2H])c([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c7ccccc7n(-c7ccccc7)c56)c([2H])c([2H])c43)cc2)c([2H])c1[2H] ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c7ccccc7n(-c7ccc([2H])cc7)c56)c([2H])c([2H])c43)cc2)cc1]

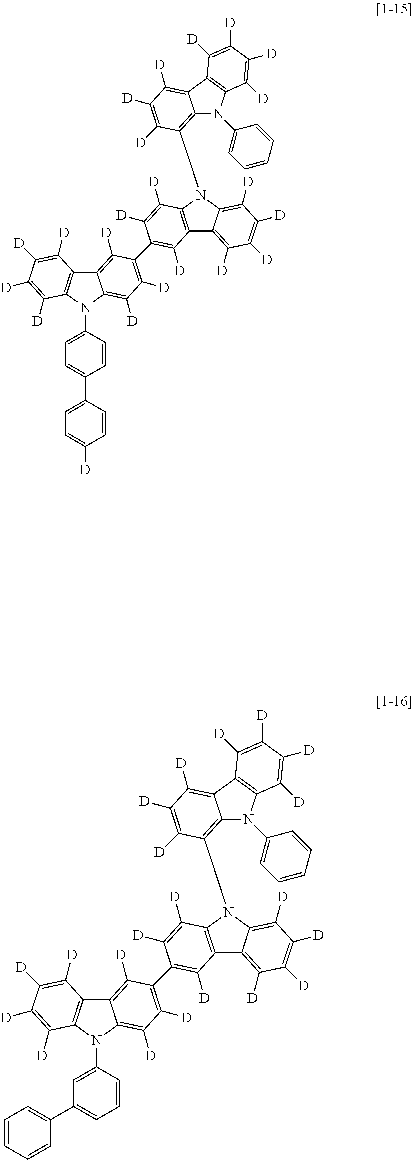 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c7c([2H])c([2H])c([2H])c([2H])c7n(-c7ccccc7)c56)c([2H])c([2H])c43)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c5c([2H])c([2H])c([2H])c([2H])c5n(-c5ccccc5)c34)c([2H])c([2H])c1n2-c1cccc(-c2ccccc2)c1]

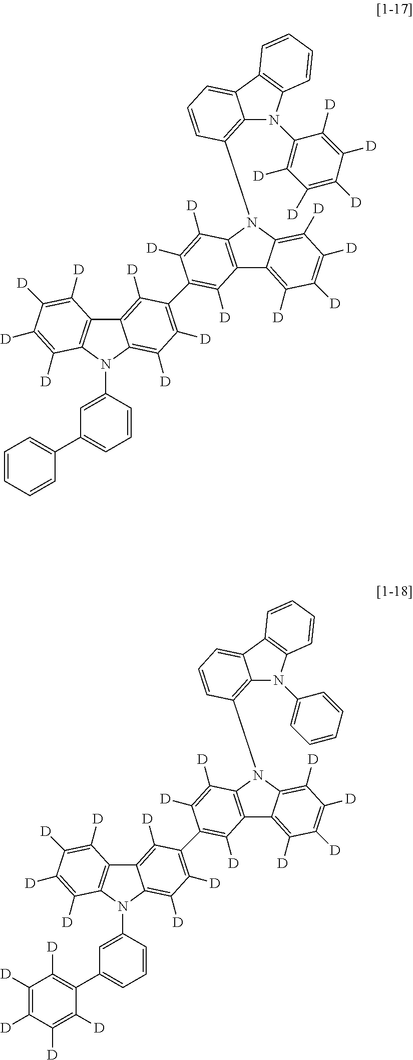 [SMILES: [2H]c1c([2H])c([2H])c(-n2c3ccccc3c3cccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6cccc(-c7ccccc7)c6)c([2H])c([2H])c54)c32)c([2H])c1[2H] ; [2H]c1c([2H])c([2H])c(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c7ccccc7n(-c7ccccc7)c56)c([2H])c([2H])c43)c2)c([2H])c1[2H]]

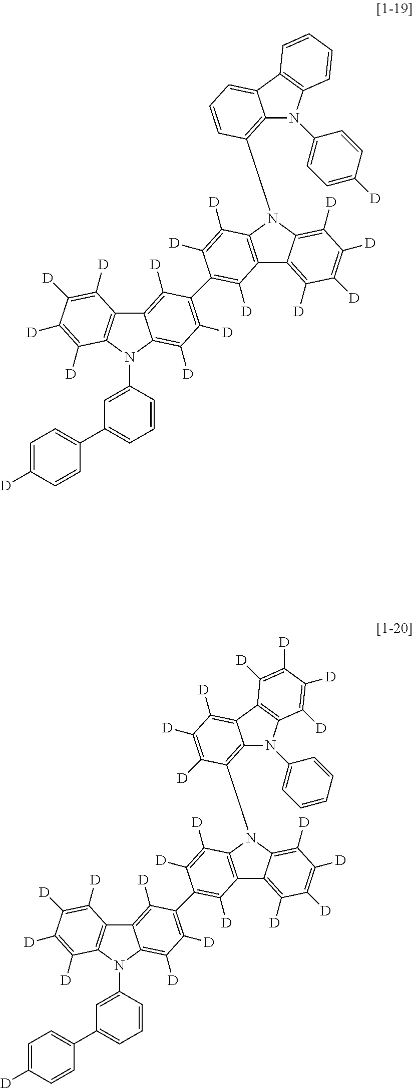 [SMILES: [2H]c1ccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c7ccccc7n(-c7ccc([2H])cc7)c56)c([2H])c([2H])c43)c2)cc1 ; [2H]c1ccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c7c([2H])c([2H])c([2H])c([2H])c7n(-c7ccccc7)c56)c([2H])c([2H])c43)c2)cc1]

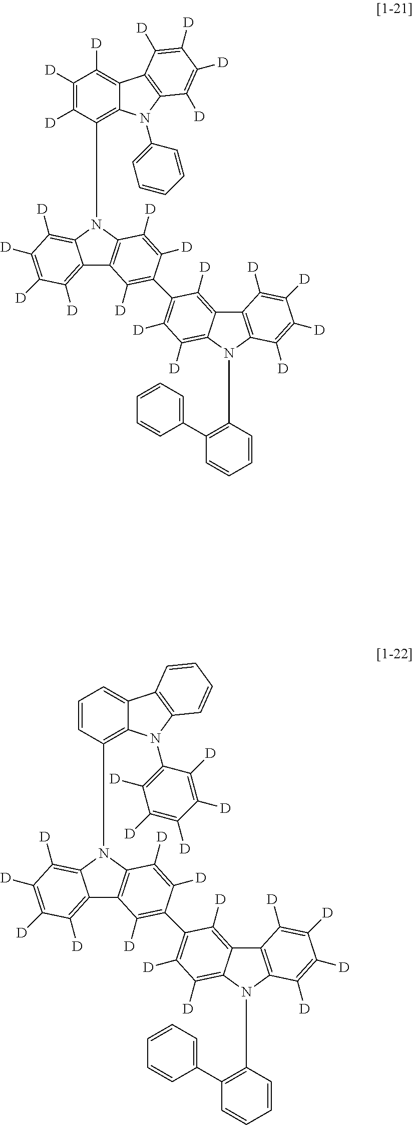 [SMILES: [2H]c1c([2H])c([2H])c2c(c1[2H])c1c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5-c5ccccc5)c([2H])c([2H])c43)c1n2-c1ccccc1 ; [2H]c1c([2H])c([2H])c(-n2c3ccccc3c3cccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6-c6ccccc6)c([2H])c([2H])c54)c32)c([2H])c1[2H]]

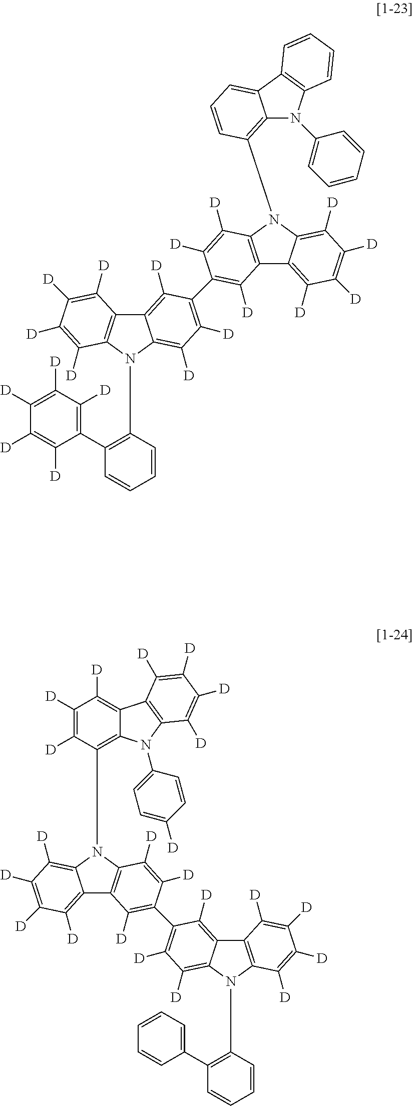 [SMILES: [2H]c1c([2H])c([2H])c(-c2ccccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c6ccccc6n(-c6ccccc6)c45)c([2H])c([2H])c32)c([2H])c1[2H] ; [2H]c1ccc(-n2c3c([2H])c([2H])c([2H])c([2H])c3c3c([2H])c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6-c6ccccc6)c([2H])c([2H])c54)c32)cc1]

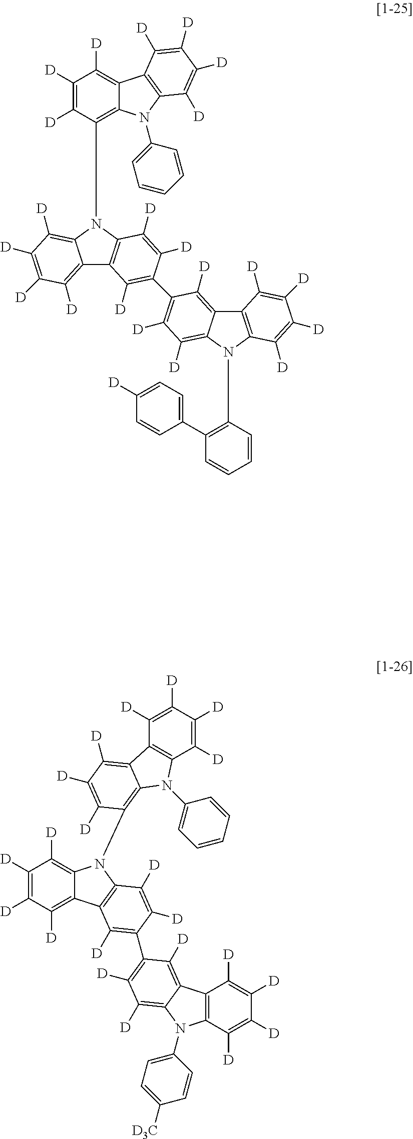 [SMILES: [2H]c1ccc(-c2ccccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c6c([2H])c([2H])c([2H])c([2H])c6n(-c6ccccc6)c45)c([2H])c([2H])c32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c5c([2H])c([2H])c([2H])c([2H])c5n(-c5ccccc5)c34)c([2H])c([2H])c1n2-c1ccc(C([2H])([2H])[2H])cc1]

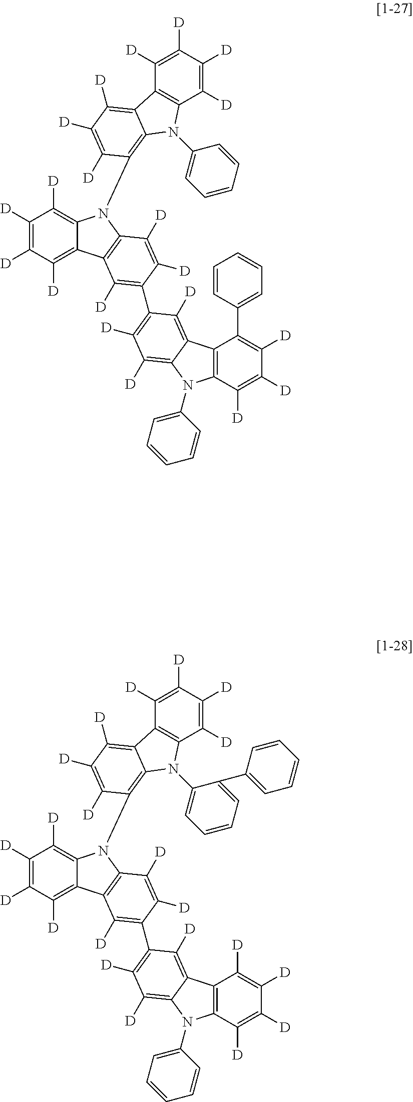 [SMILES: [2H]c1c([2H])c([2H])c2c(c1[2H])c1c([2H])c([2H])c([2H])c(-n3c4c([2H])c([2H])c([2H])c([2H])c4c4c([2H])c(-c5c([2H])c([2H])c6c(c5[2H])c5c(-c7ccccc7)c([2H])c([2H])c([2H])c5n6-c5ccccc5)c([2H])c([2H])c43)c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c5c([2H])c([2H])c([2H])c([2H])c5n(-c5ccccc5-c5ccccc5)c34)c([2H])c([2H])c1n2-c1ccccc1]

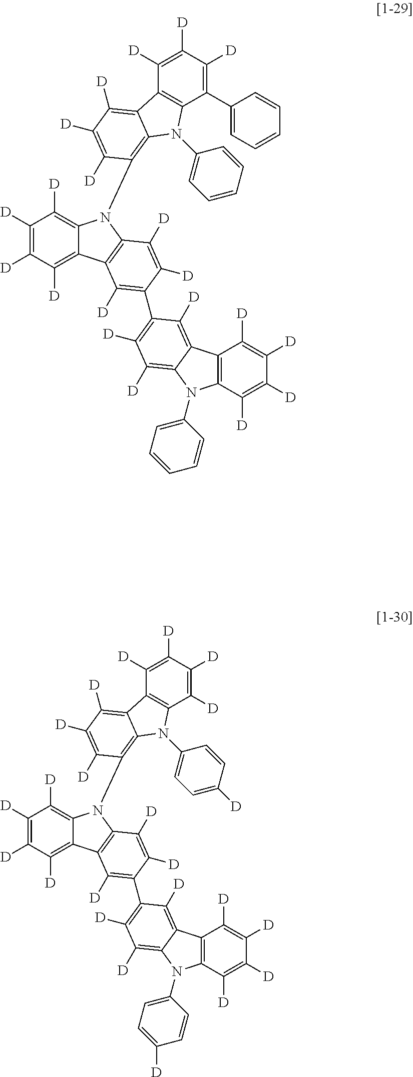 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c5c([2H])c([2H])c([2H])c(-c6ccccc6)c5n(-c5ccccc5)c34)c([2H])c([2H])c1n2-c1ccccc1 ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c6c([2H])c([2H])c([2H])c([2H])c6n(-c6ccc([2H])cc6)c45)c([2H])c([2H])c32)cc1]

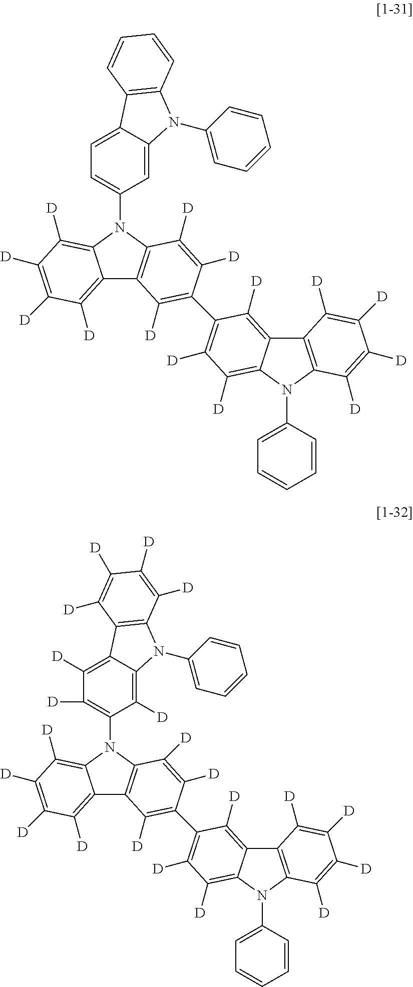 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c5ccccc5n(-c5ccccc5)c4c3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccccc1]

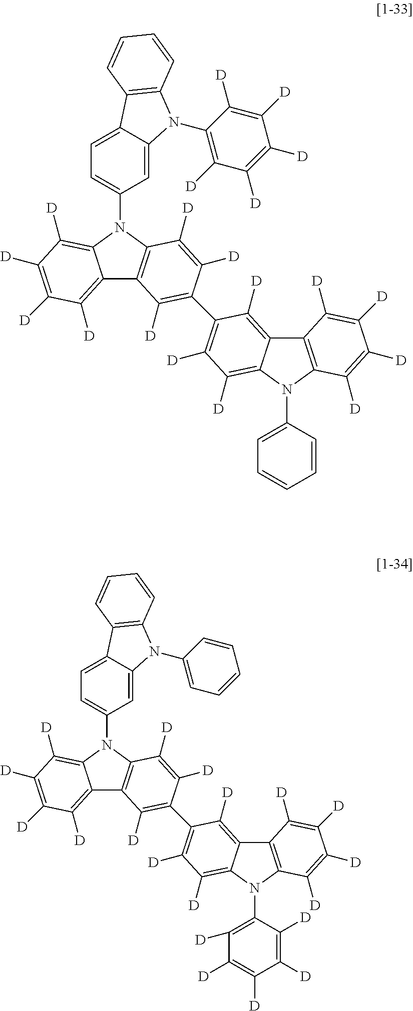 [SMILES: [2H]c1c([2H])c([2H])c(-n2c3ccccc3c3ccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)cc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccccc6n(-c6ccccc6)c5c4)c([2H])c([2H])c32)c([2H])c1[2H]]

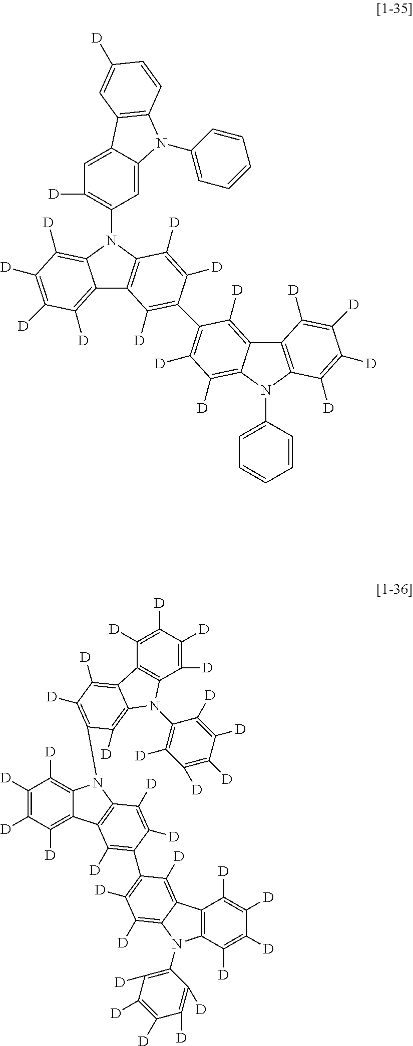 [SMILES: [2H]c1ccc2c(c1)c1cc([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5)c([2H])c([2H])c43)cc1n2-c1ccccc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c6c([2H])c([2H])c([2H])c([2H])c6n(-c6c([2H])c([2H])c([2H])c([2H])c6[2H])c5c4[2H])c([2H])c([2H])c32)c([2H])c1[2H]]

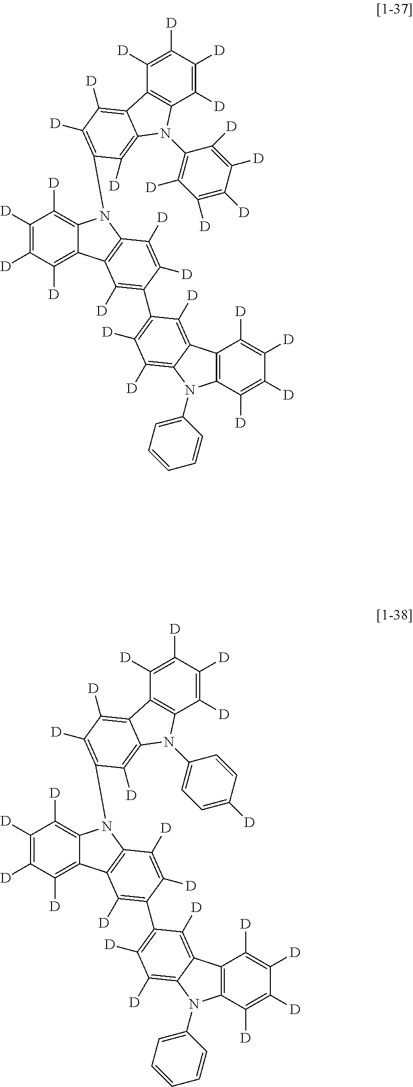 [SMILES: [2H]c1c([2H])c([2H])c(-n2c3c([2H])c([2H])c([2H])c([2H])c3c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c([2H])c32)c([2H])c1[2H] ; [2H]c1ccc(-n2c3c([2H])c([2H])c([2H])c([2H])c3c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c([2H])c32)cc1]

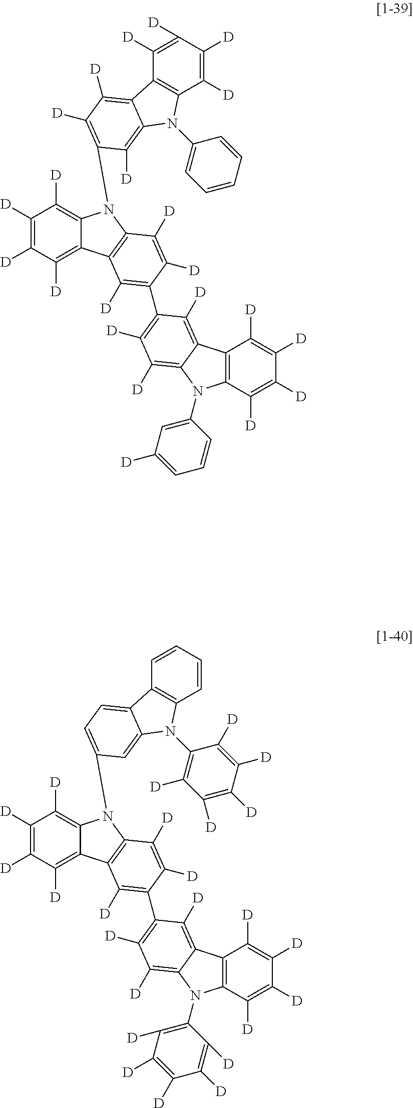 [SMILES: [2H]c1cccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c6c([2H])c([2H])c([2H])c([2H])c6n(-c6ccccc6)c5c4[2H])c([2H])c([2H])c32)c1 ; [2H]c1c([2H])c([2H])c(-n2c3ccccc3c3ccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)cc32)c([2H])c1[2H]]

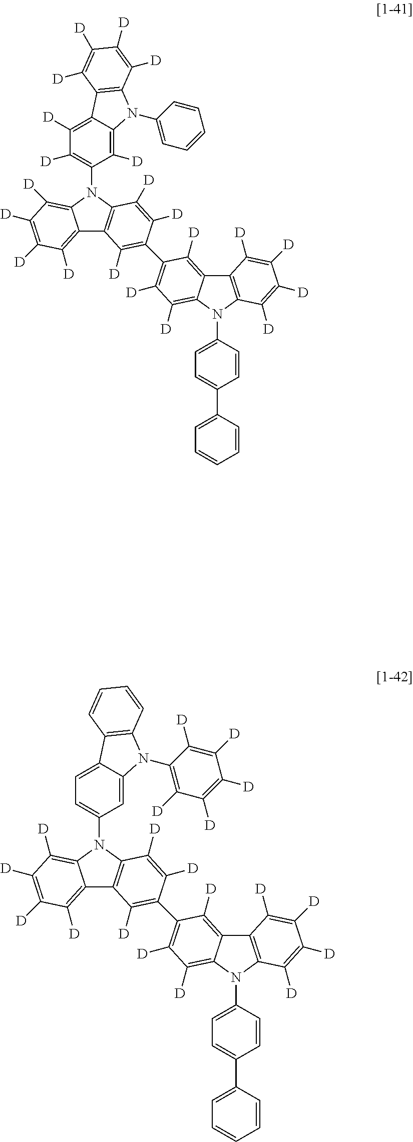 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccc(-c2ccccc2)cc1 ; [2H]c1c([2H])c([2H])c(-n2c3ccccc3c3ccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccc(-c7ccccc7)cc6)c([2H])c([2H])c54)cc32)c([2H])c1[2H]]

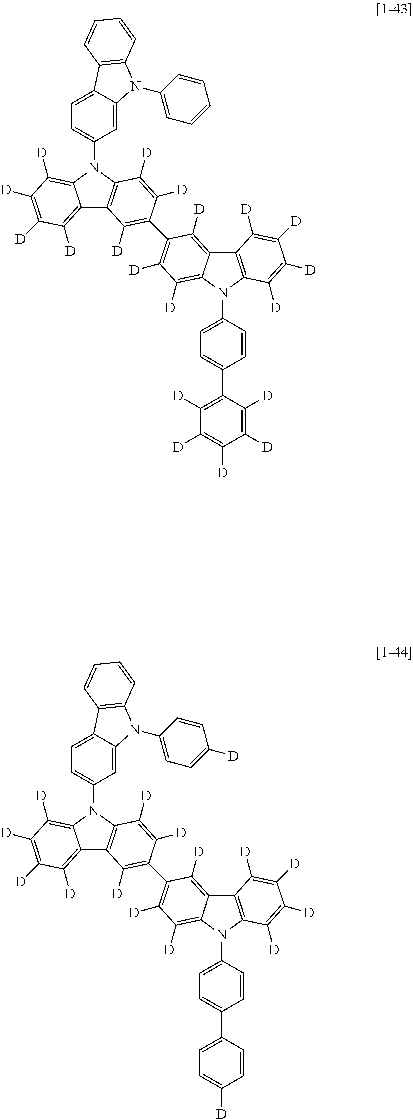 [SMILES: [2H]c1c([2H])c([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c7ccccc7n(-c7ccccc7)c6c5)c([2H])c([2H])c43)cc2)c([2H])c1[2H] ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c7ccccc7n(-c7ccc([2H])cc7)c6c5)c([2H])c([2H])c43)cc2)cc1]

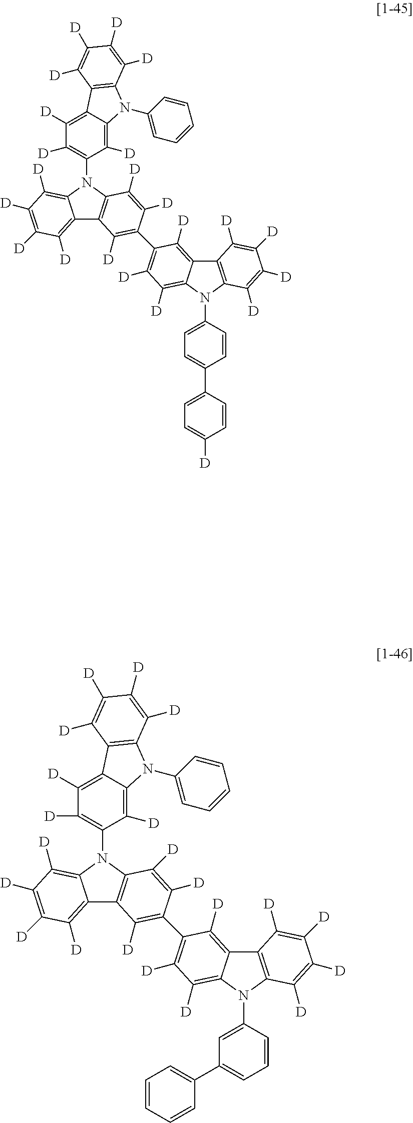 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c7c([2H])c([2H])c([2H])c([2H])c7n(-c7ccccc7)c6c5[2H])c([2H])c([2H])c43)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1cccc(-c2ccccc2)c1]

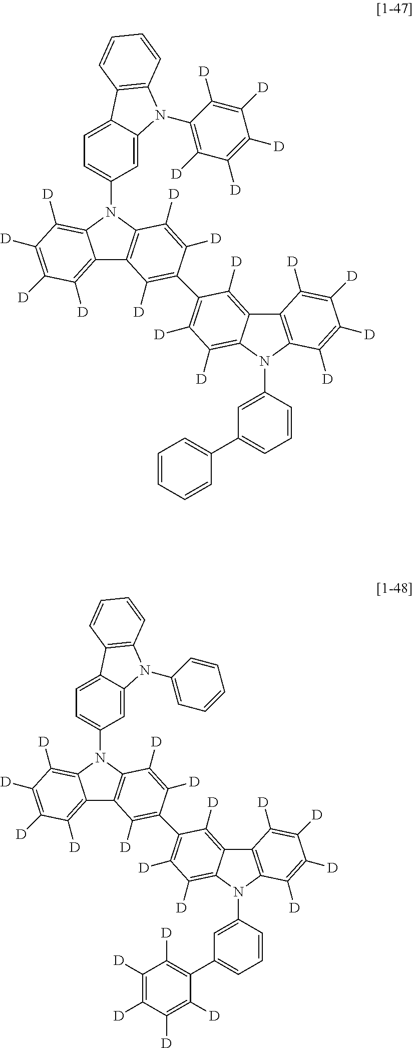 [SMILES: [2H]c1c([2H])c([2H])c(-n2c3ccccc3c3ccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6cccc(-c7ccccc7)c6)c([2H])c([2H])c54)cc32)c([2H])c1[2H] ; [2H]c1c([2H])c([2H])c(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c7ccccc7n(-c7ccccc7)c6c5)c([2H])c([2H])c43)c2)c([2H])c1[2H]]

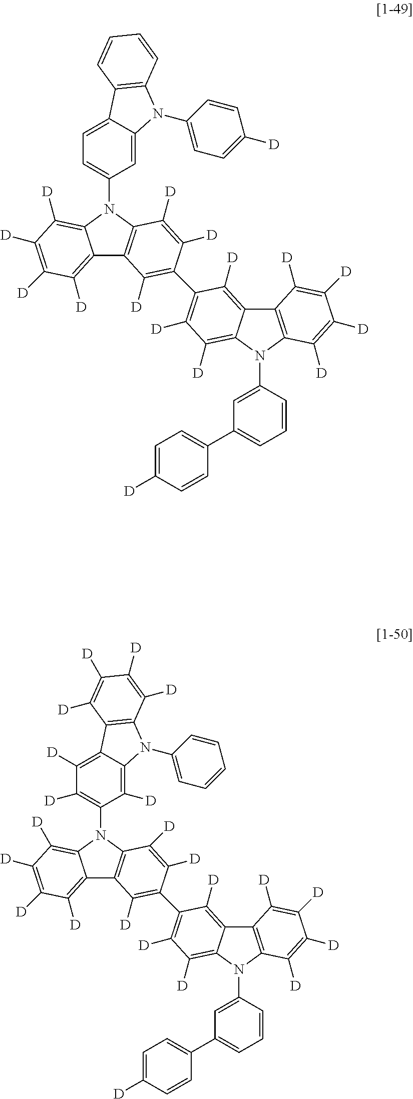 [SMILES: [2H]c1ccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c7ccccc7n(-c7ccc([2H])cc7)c6c5)c([2H])c([2H])c43)c2)cc1 ; [2H]c1ccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c7c([2H])c([2H])c([2H])c([2H])c7n(-c7ccccc7)c6c5[2H])c([2H])c([2H])c43)c2)cc1]

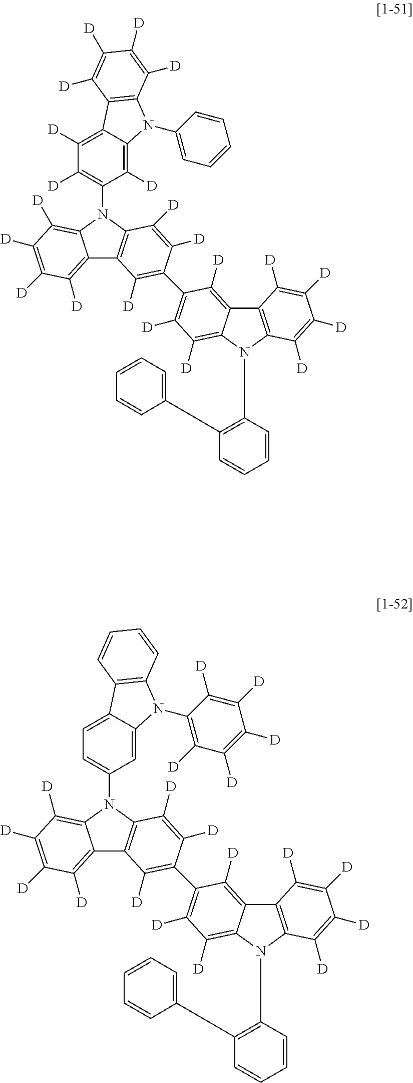 [SMILES: [2H]c1c([2H])c([2H])c2c(c1[2H])c1c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5-c5ccccc5)c([2H])c([2H])c43)c([2H])c1n2-c1ccccc1 ; [2H]c1c([2H])c([2H])c(-n2c3ccccc3c3ccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6-c6ccccc6)c([2H])c([2H])c54)cc32)c([2H])c1[2H]]

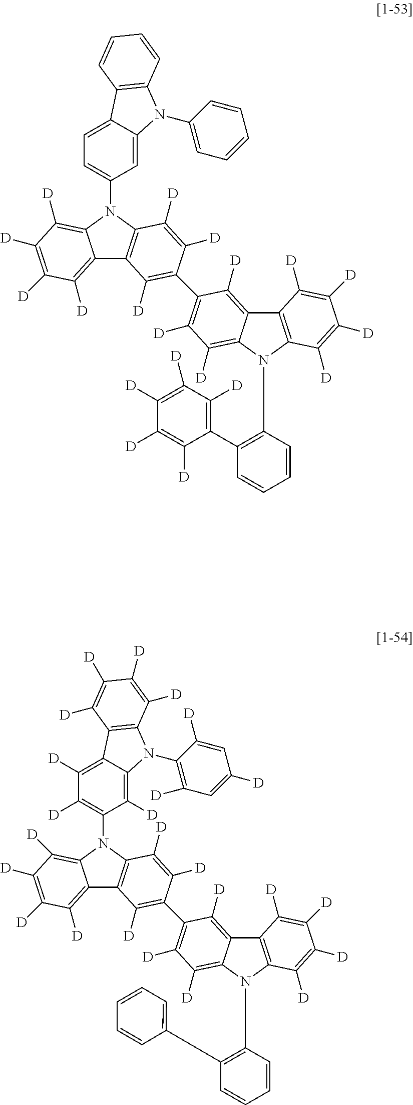 [SMILES: [2H]c1c([2H])c([2H])c(-c2ccccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c6ccccc6n(-c6ccccc6)c5c4)c([2H])c([2H])c32)c([2H])c1[2H] ; [2H]c1cc([2H])c(-n2c3c([2H])c([2H])c([2H])c([2H])c3c3c([2H])c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6-c6ccccc6)c([2H])c([2H])c54)c([2H])c32)c([2H])c1]

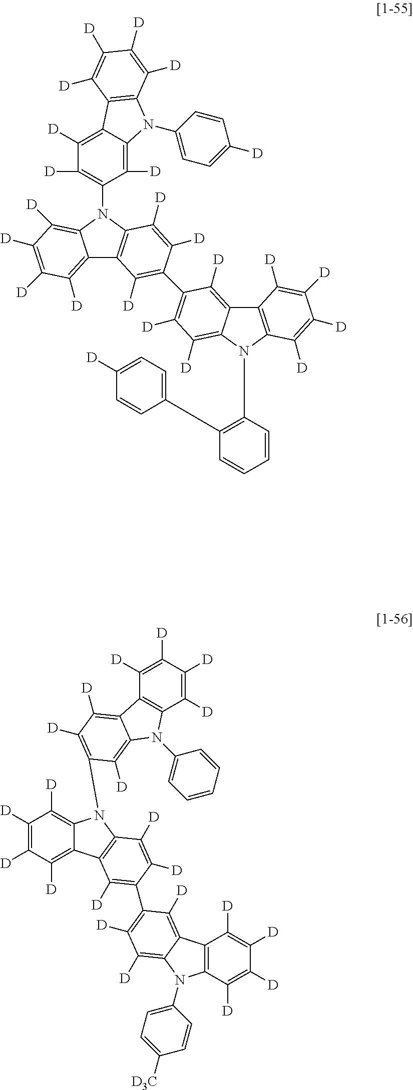 [SMILES: [2H]c1ccc(-c2ccccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c6c([2H])c([2H])c([2H])c([2H])c6n(-c6ccc([2H])cc6)c5c4[2H])c([2H])c([2H])c32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5)c4c3[2H])c([2H])c([2H])c1n2-c1ccc(C([2H])([2H])[2H])cc1]

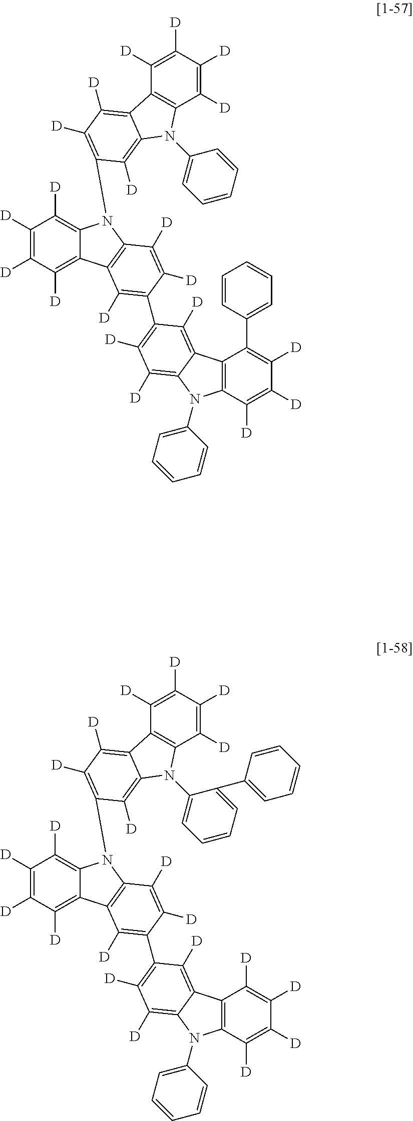 [SMILES: [2H]c1c([2H])c([2H])c2c(c1[2H])c1c([2H])c([2H])c(-n3c4c([2H])c([2H])c([2H])c([2H])c4c4c([2H])c(-c5c([2H])c([2H])c6c(c5[2H])c5c(-c7ccccc7)c([2H])c([2H])c([2H])c5n6-c5ccccc5)c([2H])c([2H])c43)c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c([2H])c5n(-c5ccccc5-c5ccccc5)c4c3[2H])c([2H])c([2H])c1n2-c1ccccc1]

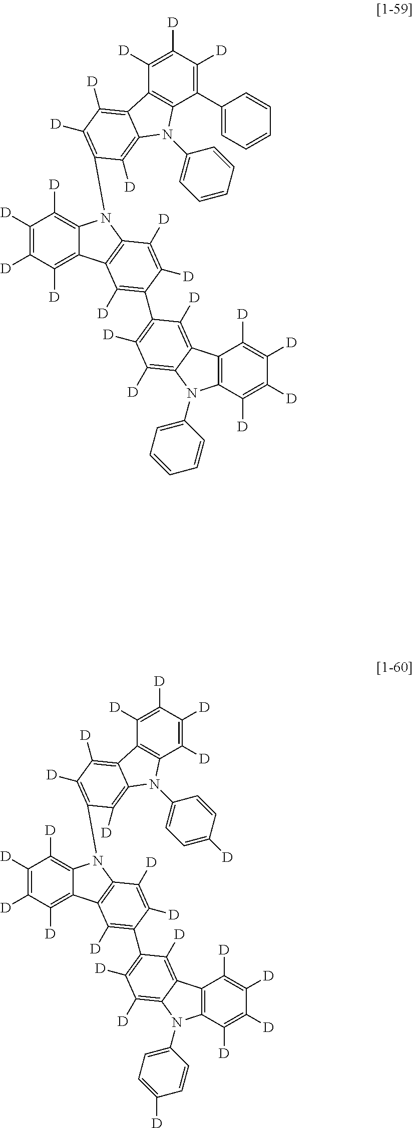 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c5c([2H])c([2H])c([2H])c(-c6ccccc6)c5n(-c5ccccc5)c4c3[2H])c([2H])c([2H])c1n2-c1ccccc1 ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c6c([2H])c([2H])c([2H])c([2H])c6n(-c6ccc([2H])cc6)c5c4[2H])c([2H])c([2H])c32)cc1]

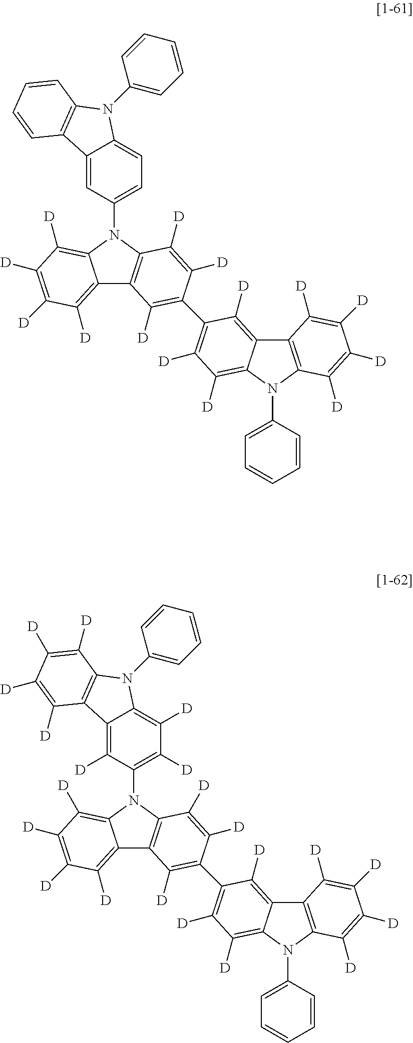 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)c3ccccc3n4-c3ccccc3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccccc1]

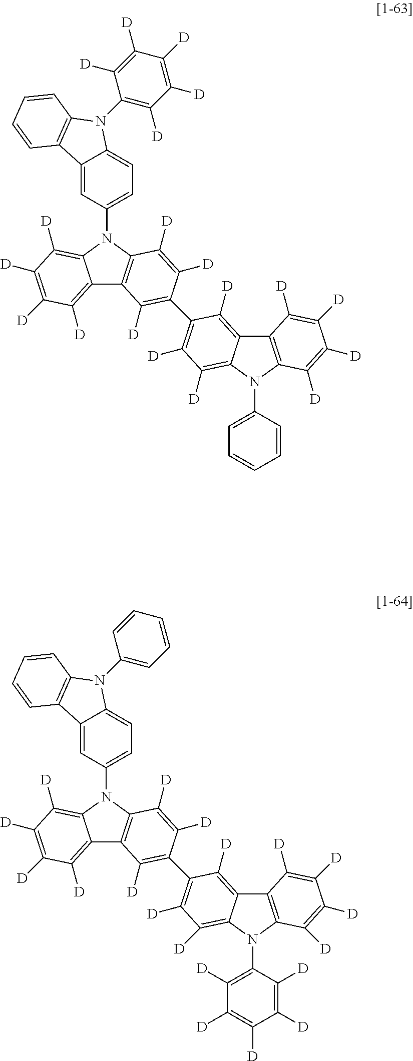 [SMILES: [2H]c1c([2H])c([2H])c(-n2c3ccccc3c3cc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)c4ccccc4n5-c4ccccc4)c([2H])c([2H])c32)c([2H])c1[2H]]

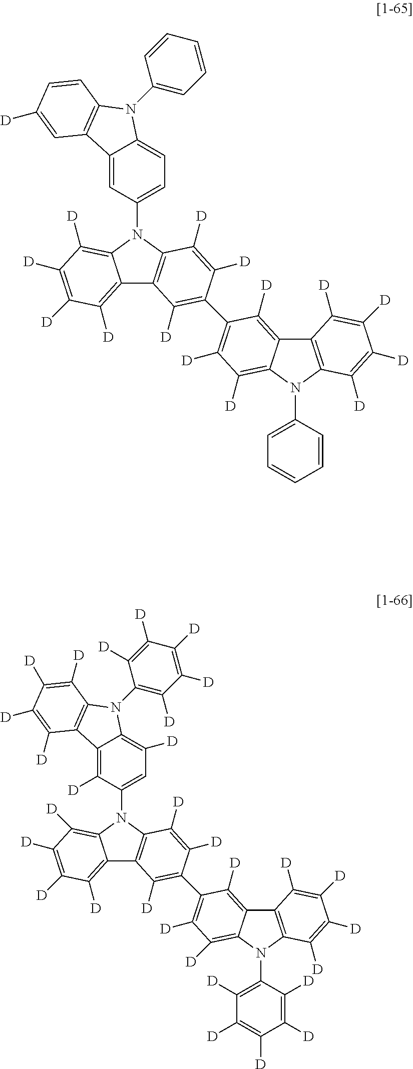 [SMILES: [2H]c1ccc2c(c1)c1cc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5)c([2H])c([2H])c43)ccc1n2-c1ccccc1 ; [2H]c1c([2H])c([2H])c(-n2c3c([2H])cc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)c([2H])c3c3c([2H])c([2H])c([2H])c([2H])c32)c([2H])c1[2H]]

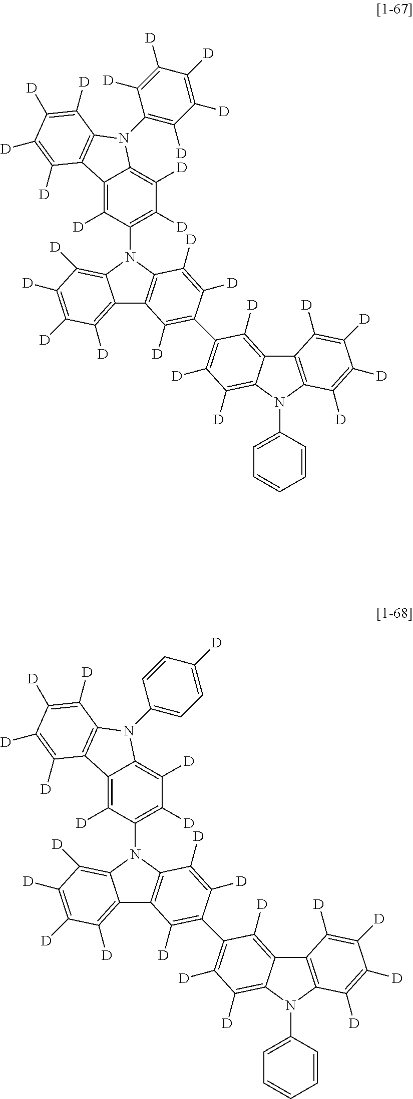 [SMILES: [2H]c1c([2H])c([2H])c(-n2c3c([2H])c([2H])c([2H])c([2H])c3c3c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c([2H])c([2H])c32)c([2H])c1[2H] ; [2H]c1ccc(-n2c3c([2H])c([2H])c([2H])c([2H])c3c3c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c([2H])c([2H])c32)cc1]

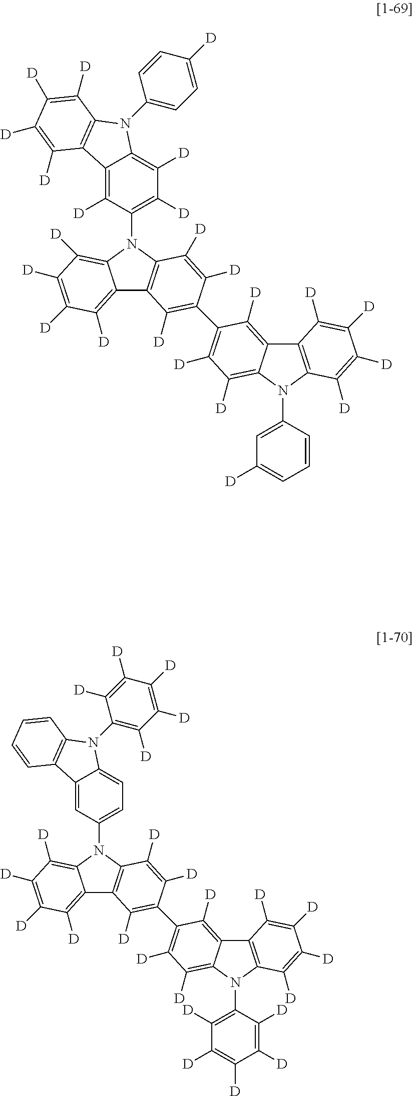 [SMILES: [2H]c1ccc(-n2c3c([2H])c([2H])c([2H])c([2H])c3c3c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6cccc([2H])c6)c([2H])c([2H])c54)c([2H])c([2H])c32)cc1 ; [2H]c1c([2H])c([2H])c(-n2c3ccccc3c3cc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)ccc32)c([2H])c1[2H]]

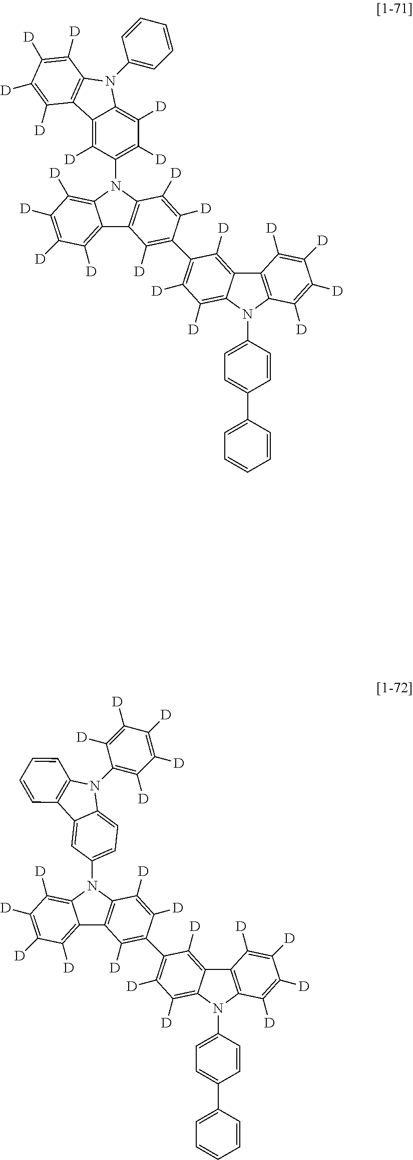 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccc(-c2ccccc2)cc1 ; [2H]c1c([2H])c([2H])c(-n2c3ccccc3c3cc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccc(-c7ccccc7)cc6)c([2H])c([2H])c54)ccc32)c([2H])c1[2H]]

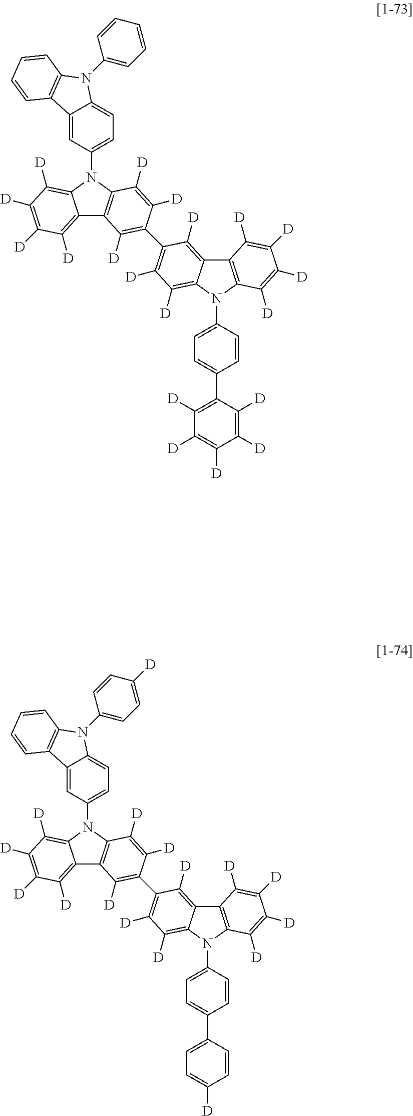 [SMILES: [2H]c1c([2H])c([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)c5ccccc5n6-c5ccccc5)c([2H])c([2H])c43)cc2)c([2H])c1[2H] ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)c5ccccc5n6-c5ccc([2H])cc5)c([2H])c([2H])c43)cc2)cc1]

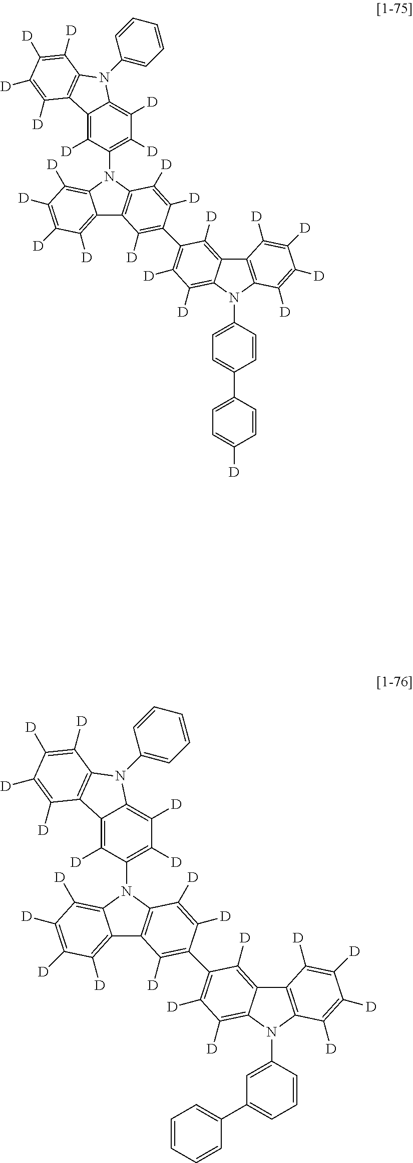 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(c5[2H])c5c([2H])c([2H])c([2H])c([2H])c5n6-c5ccccc5)c([2H])c([2H])c43)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1cccc(-c2ccccc2)c1]

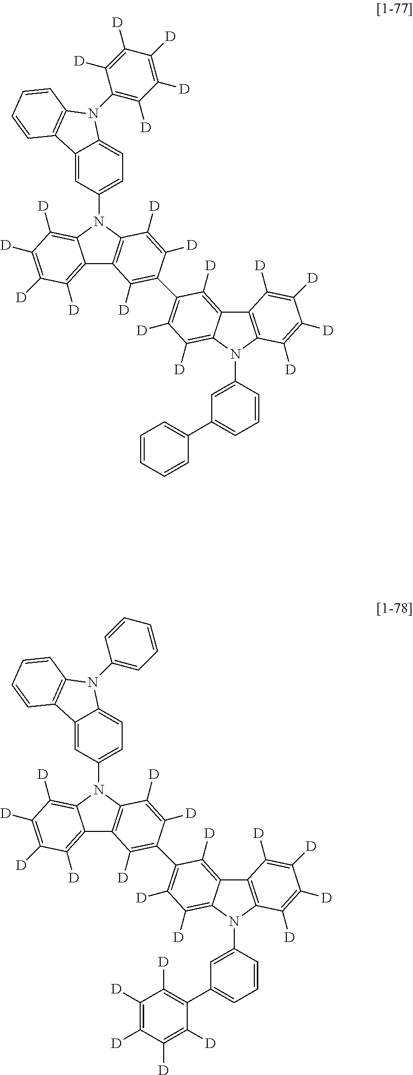 [SMILES: [2H]c1c([2H])c([2H])c(-n2c3ccccc3c3cc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6cccc(-c7ccccc7)c6)c([2H])c([2H])c54)ccc32)c([2H])c1[2H] ; [2H]c1c([2H])c([2H])c(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)c5ccccc5n6-c5ccccc5)c([2H])c([2H])c43)c2)c([2H])c1[2H]]

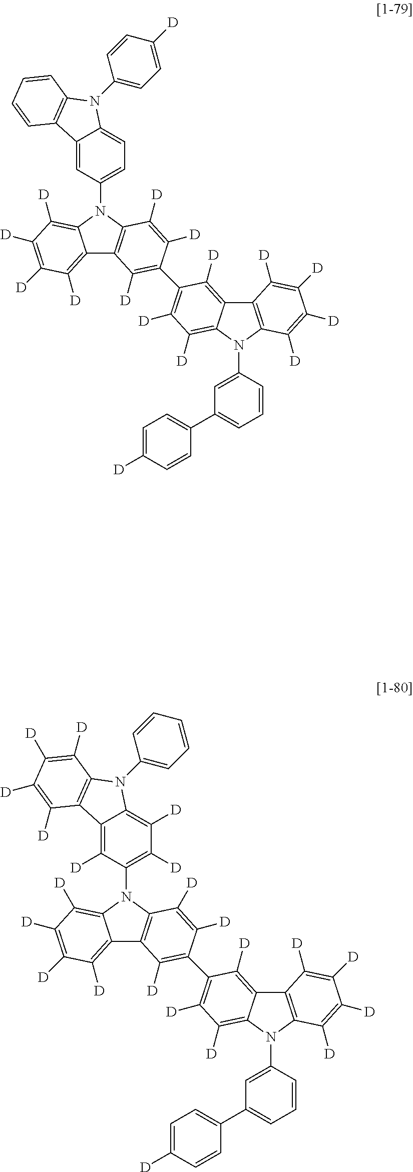 [SMILES: [2H]c1ccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)c5ccccc5n6-c5ccc([2H])cc5)c([2H])c([2H])c43)c2)cc1 ; [2H]c1ccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(c5[2H])c5c([2H])c([2H])c([2H])c([2H])c5n6-c5ccccc5)c([2H])c([2H])c43)c2)cc1]

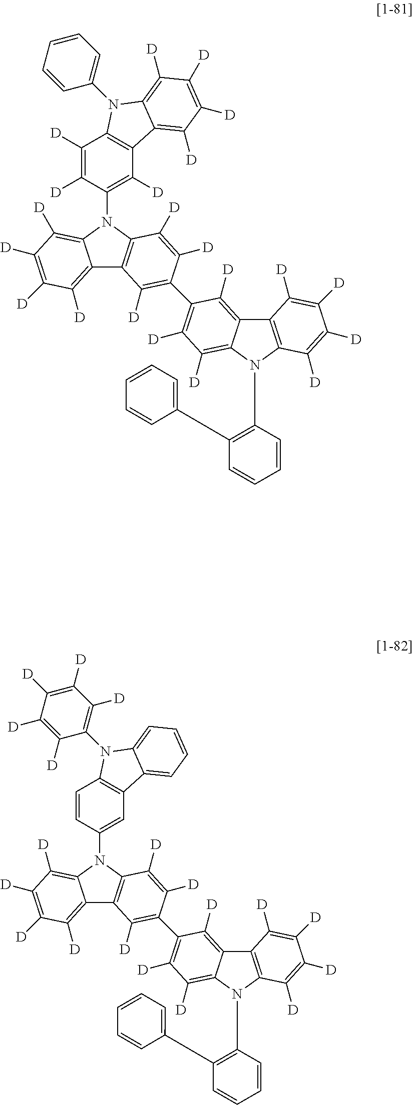 [SMILES: [2H]c1c([2H])c([2H])c2c(c1[2H])c1c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5-c5ccccc5)c([2H])c([2H])c43)c([2H])c([2H])c1n2-c1ccccc1 ; [2H]c1c([2H])c([2H])c(-n2c3ccccc3c3cc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6-c6ccccc6)c([2H])c([2H])c54)ccc32)c([2H])c1[2H]]

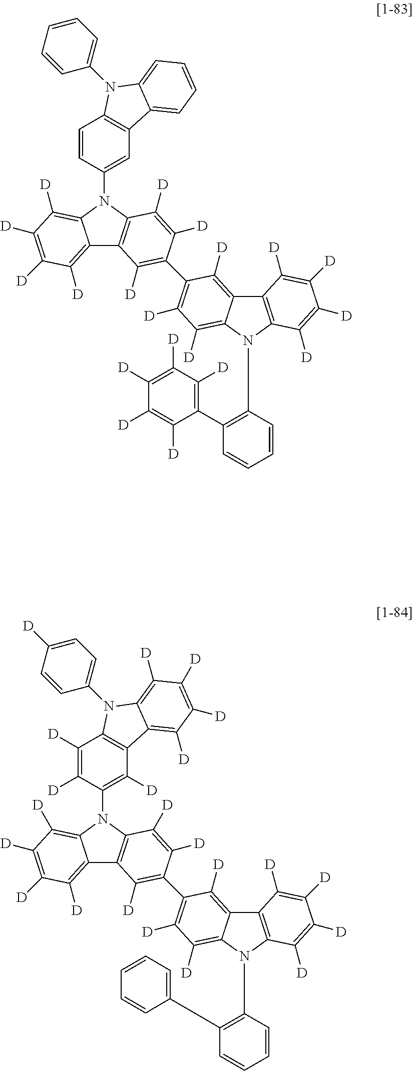 [SMILES: [2H]c1c([2H])c([2H])c(-c2ccccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)c4ccccc4n5-c4ccccc4)c([2H])c([2H])c32)c([2H])c1[2H] ; [2H]c1ccc(-n2c3c([2H])c([2H])c([2H])c([2H])c3c3c([2H])c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6-c6ccccc6)c([2H])c([2H])c54)c([2H])c([2H])c32)cc1]

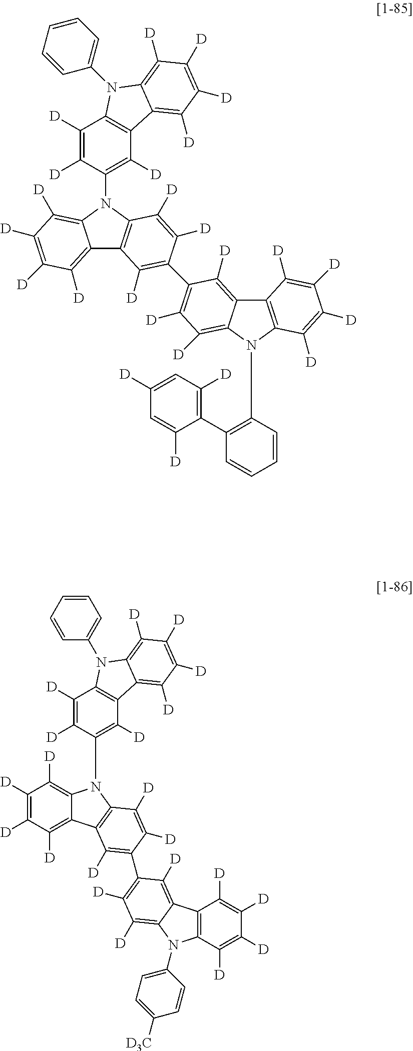 [SMILES: [2H]c1cc([2H])c(-c2ccccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(c4[2H])c4c([2H])c([2H])c([2H])c([2H])c4n5-c4ccccc4)c([2H])c([2H])c32)c([2H])c1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1ccc(C([2H])([2H])[2H])cc1]

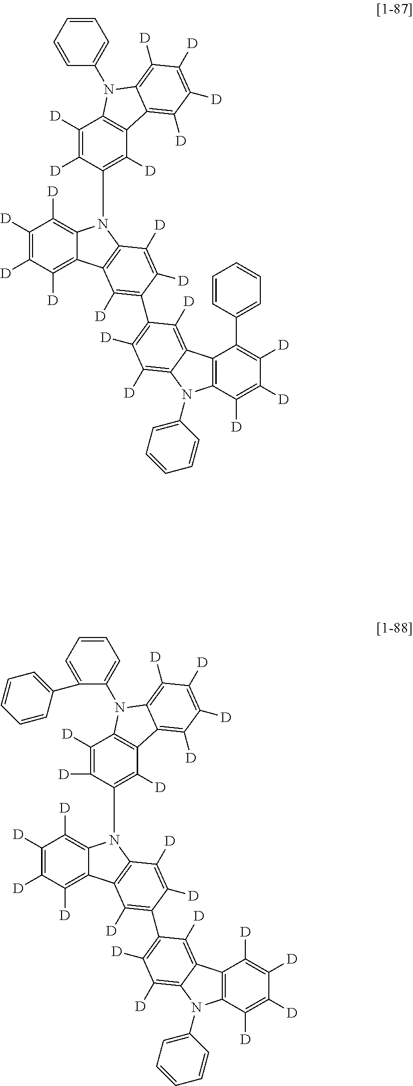 [SMILES: [2H]c1c([2H])c([2H])c2c(c1[2H])c1c([2H])c(-n3c4c([2H])c([2H])c([2H])c([2H])c4c4c([2H])c(-c5c([2H])c([2H])c6c(c5[2H])c5c(-c7ccccc7)c([2H])c([2H])c([2H])c5n6-c5ccccc5)c([2H])c([2H])c43)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3-c3ccccc3)c([2H])c([2H])c1n2-c1ccccc1]

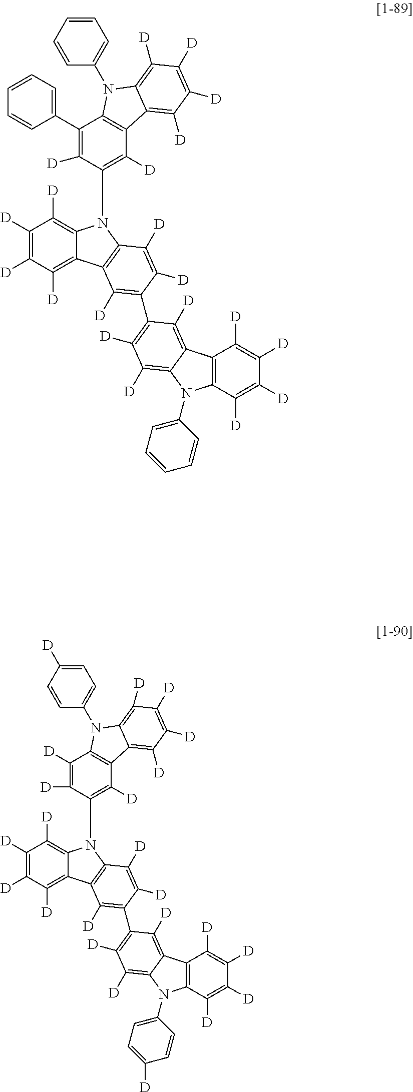 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c(-c4ccccc4)c4c(c3[2H])c3c([2H])c([2H])c([2H])c([2H])c3n4-c3ccccc3)c([2H])c([2H])c1n2-c1ccccc1 ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(c4[2H])c4c([2H])c([2H])c([2H])c([2H])c4n5-c4ccc([2H])cc4)c([2H])c([2H])c32)cc1]

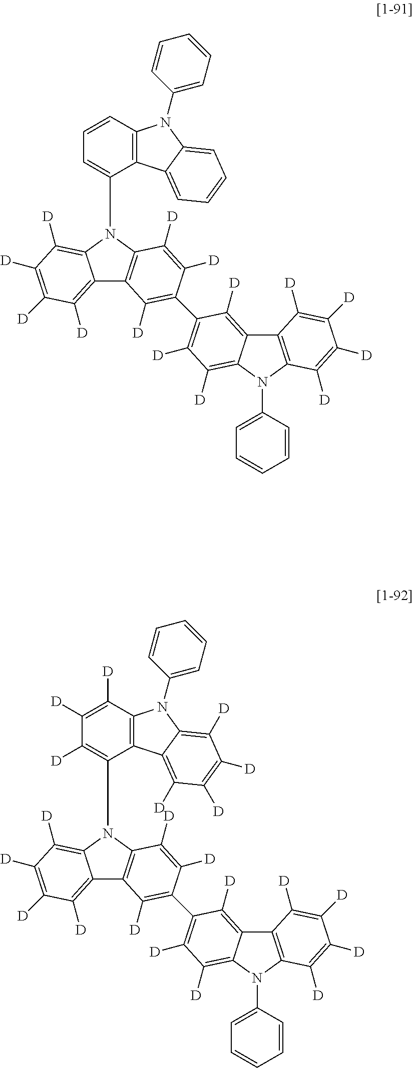 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3c3ccccc3n4-c3ccccc3)c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c3c3c([2H])c([2H])c([2H])c([2H])c3n4-c3ccccc3)c([2H])c([2H])c1n2-c1ccccc1]

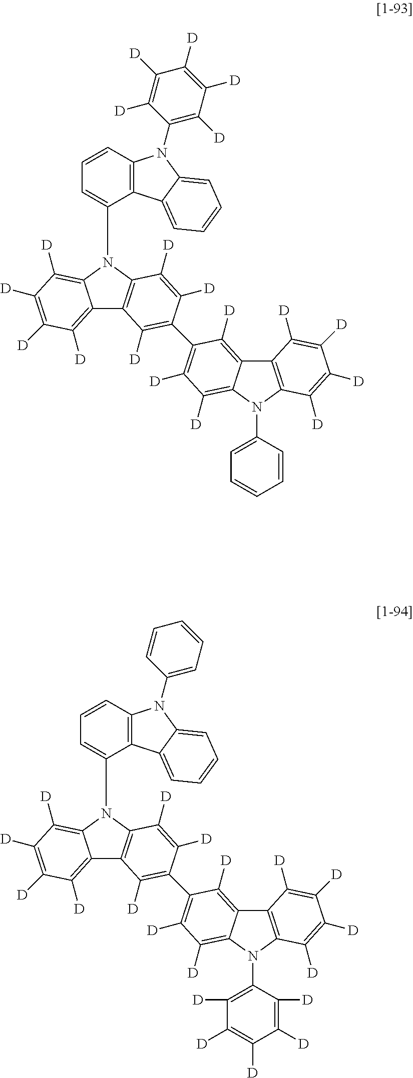 [SMILES: [2H]c1c([2H])c([2H])c(-n2c3ccccc3c3c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)cccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4c4ccccc4n5-c4ccccc4)c([2H])c([2H])c32)c([2H])c1[2H]]

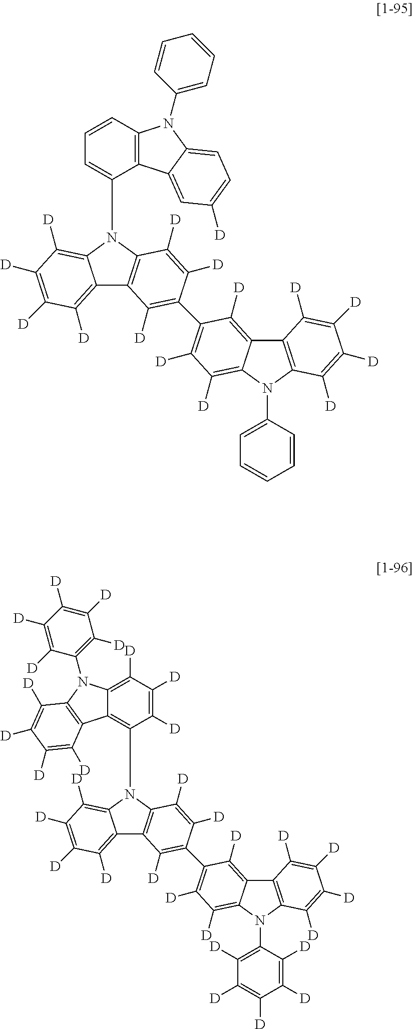 [SMILES: [2H]c1ccc2c(c1)c1c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5)c([2H])c([2H])c43)cccc1n2-c1ccccc1 ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c4c4c([2H])c([2H])c([2H])c([2H])c4n5-c4c([2H])c([2H])c([2H])c([2H])c4[2H])c([2H])c([2H])c32)c([2H])c1[2H]]

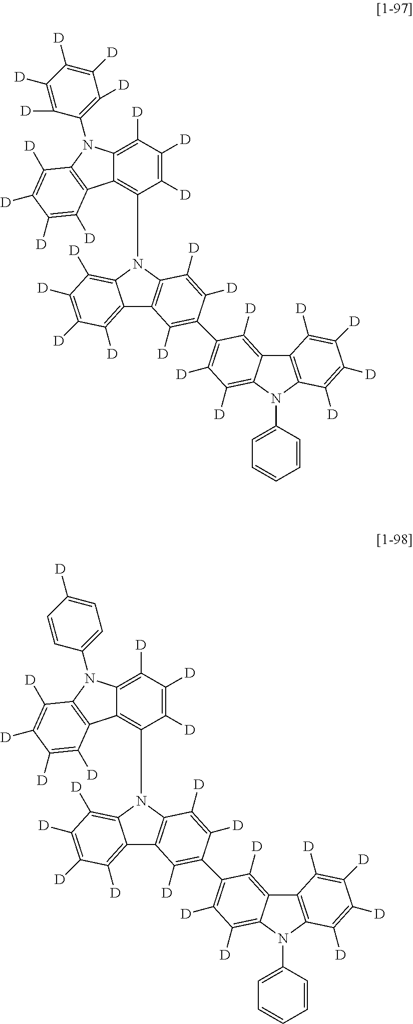 [SMILES: [2H]c1c([2H])c([2H])c(-n2c3c([2H])c([2H])c([2H])c([2H])c3c3c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c([2H])c([2H])c([2H])c32)c([2H])c1[2H] ; [2H]c1ccc(-n2c3c([2H])c([2H])c([2H])c([2H])c3c3c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)c([2H])c([2H])c([2H])c32)cc1]

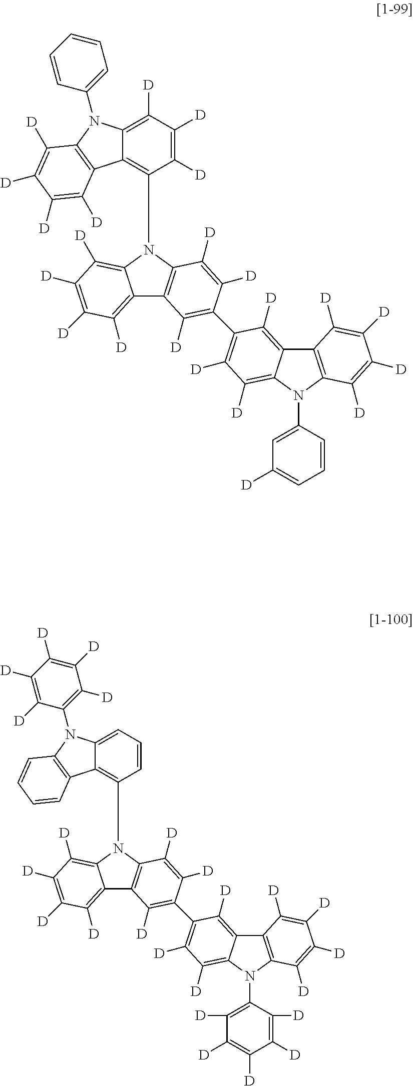 [SMILES: [2H]c1cccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c4c4c([2H])c([2H])c([2H])c([2H])c4n5-c4ccccc4)c([2H])c([2H])c32)c1 ; [2H]c1c([2H])c([2H])c(-n2c3ccccc3c3c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6c([2H])c([2H])c([2H])c([2H])c6[2H])c([2H])c([2H])c54)cccc32)c([2H])c1[2H]]

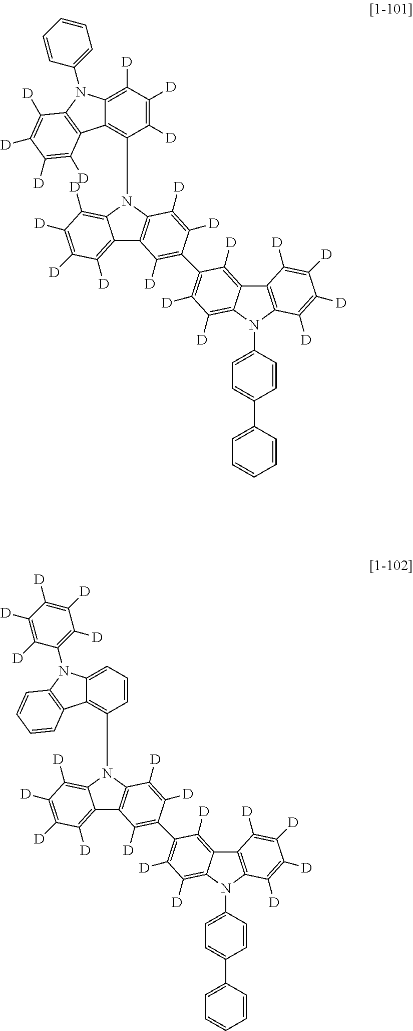 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c3c3c([2H])c([2H])c([2H])c([2H])c3n4-c3ccccc3)c([2H])c([2H])c1n2-c1ccc(-c2ccccc2)cc1 ; [2H]c1c([2H])c([2H])c(-n2c3ccccc3c3c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccc(-c7ccccc7)cc6)c([2H])c([2H])c54)cccc32)c([2H])c1[2H]]

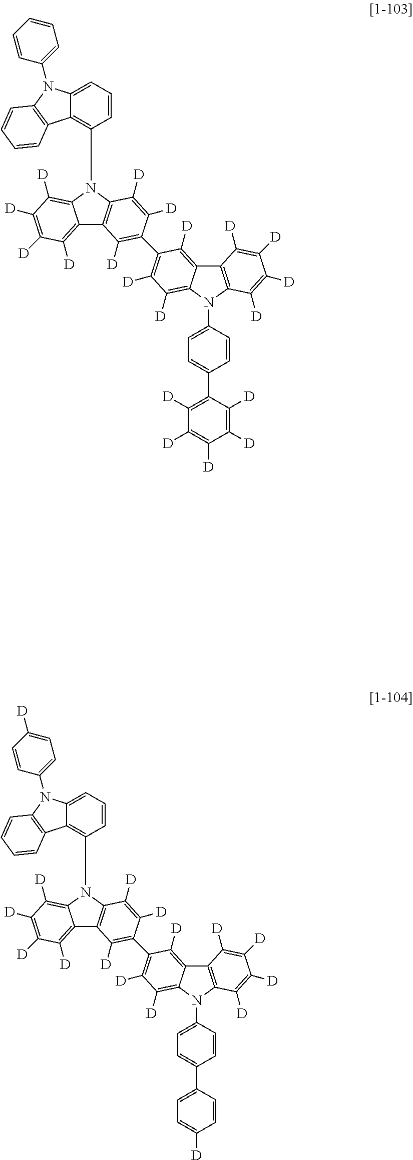 [SMILES: [2H]c1c([2H])c([2H])c(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5c5ccccc5n6-c5ccccc5)c([2H])c([2H])c43)cc2)c([2H])c1[2H] ; [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5c5ccccc5n6-c5ccc([2H])cc5)c([2H])c([2H])c43)cc2)cc1]

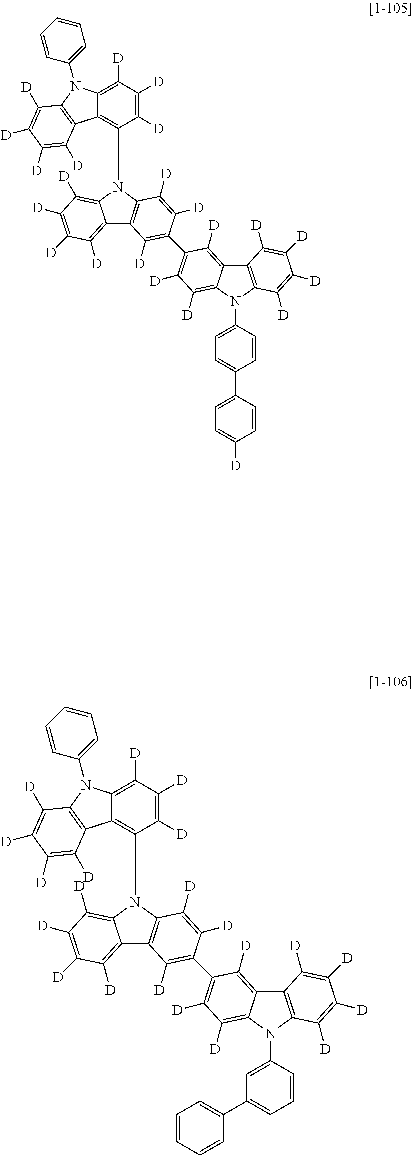 [SMILES: [2H]c1ccc(-c2ccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c5c5c([2H])c([2H])c([2H])c([2H])c5n6-c5ccccc5)c([2H])c([2H])c43)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c3c3c([2H])c([2H])c([2H])c([2H])c3n4-c3ccccc3)c([2H])c([2H])c1n2-c1cccc(-c2ccccc2)c1]

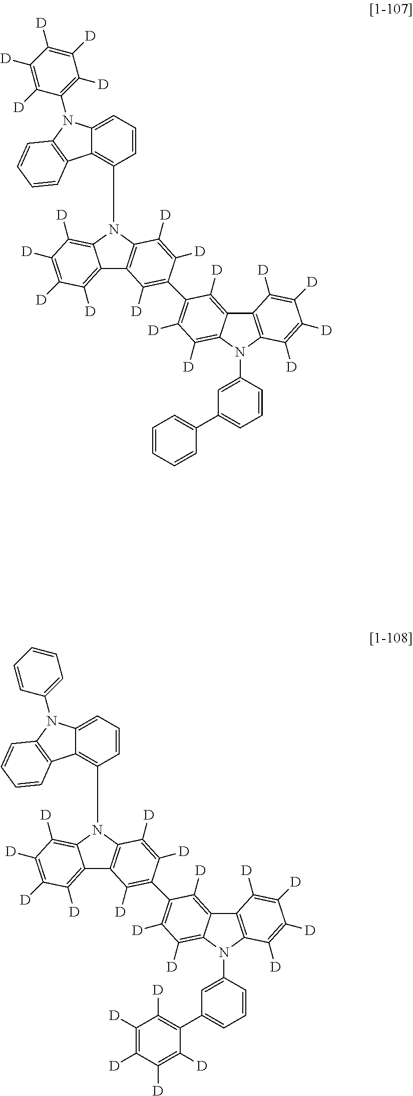 [SMILES: [2H]c1c([2H])c([2H])c(-n2c3ccccc3c3c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6cccc(-c7ccccc7)c6)c([2H])c([2H])c54)cccc32)c([2H])c1[2H] ; [2H]c1c([2H])c([2H])c(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5c5ccccc5n6-c5ccccc5)c([2H])c([2H])c43)c2)c([2H])c1[2H]]

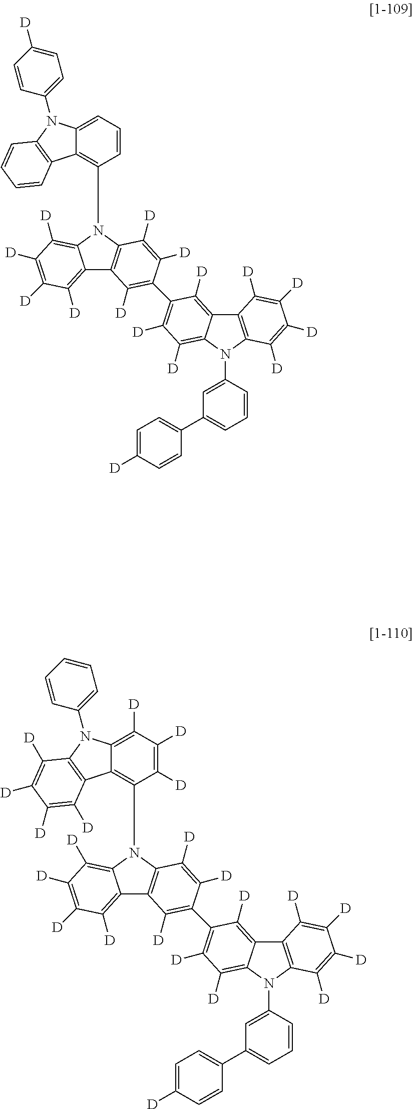 [SMILES: [2H]c1ccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5c5ccccc5n6-c5ccc([2H])cc5)c([2H])c([2H])c43)c2)cc1 ; [2H]c1ccc(-c2cccc(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c5c5c([2H])c([2H])c([2H])c([2H])c5n6-c5ccccc5)c([2H])c([2H])c43)c2)cc1]

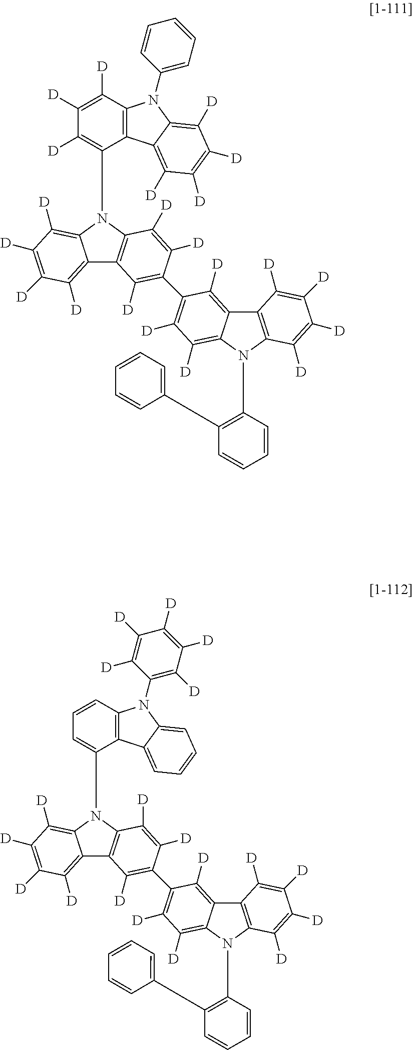 [SMILES: [2H]c1c([2H])c([2H])c2c(c1[2H])c1c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccccc5-c5ccccc5)c([2H])c([2H])c43)c([2H])c([2H])c([2H])c1n2-c1ccccc1 ; [2H]c1c([2H])c([2H])c(-n2c3ccccc3c3c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6-c6ccccc6)c([2H])c([2H])c54)cccc32)c([2H])c1[2H]]

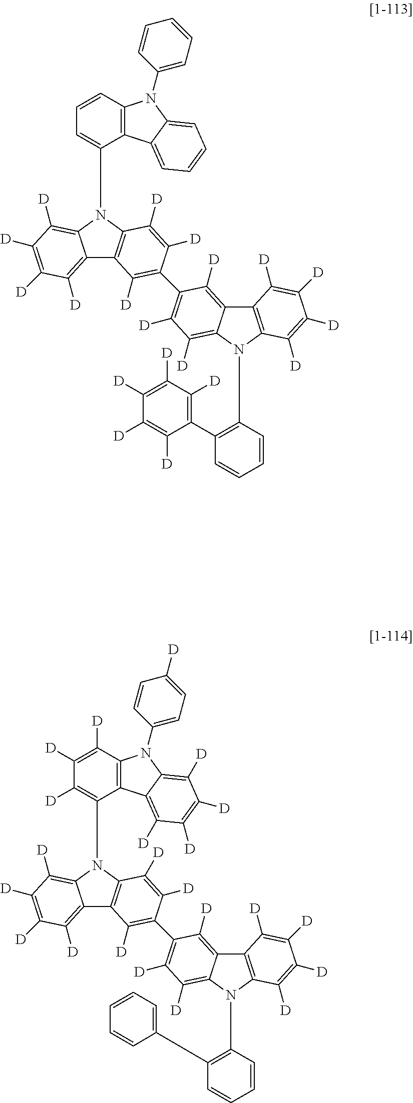 [SMILES: [2H]c1c([2H])c([2H])c(-c2ccccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4c4ccccc4n5-c4ccccc4)c([2H])c([2H])c32)c([2H])c1[2H] ; [2H]c1ccc(-n2c3c([2H])c([2H])c([2H])c([2H])c3c3c(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6-c6ccccc6)c([2H])c([2H])c54)c([2H])c([2H])c([2H])c32)cc1]

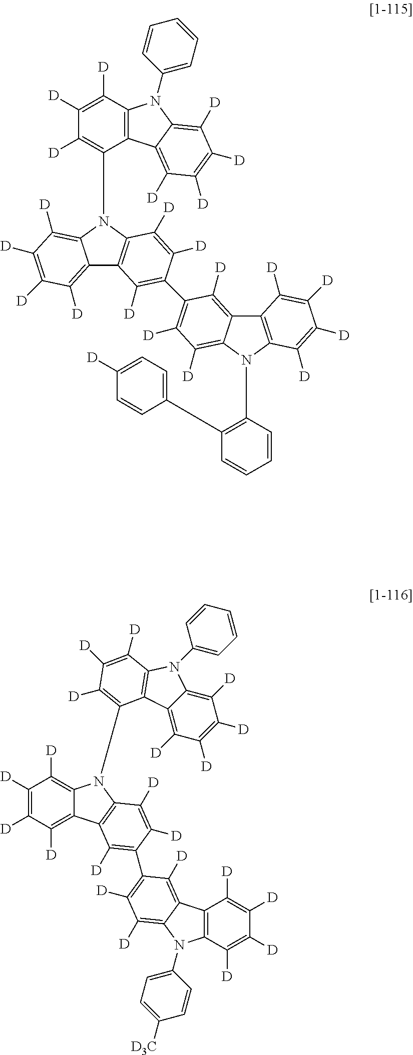 [SMILES: [2H]c1ccc(-c2ccccc2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c4c4c([2H])c([2H])c([2H])c([2H])c4n5-c4ccccc4)c([2H])c([2H])c32)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c3c3c([2H])c([2H])c([2H])c([2H])c3n4-c3ccccc3)c([2H])c([2H])c1n2-c1ccc(C([2H])([2H])[2H])cc1]

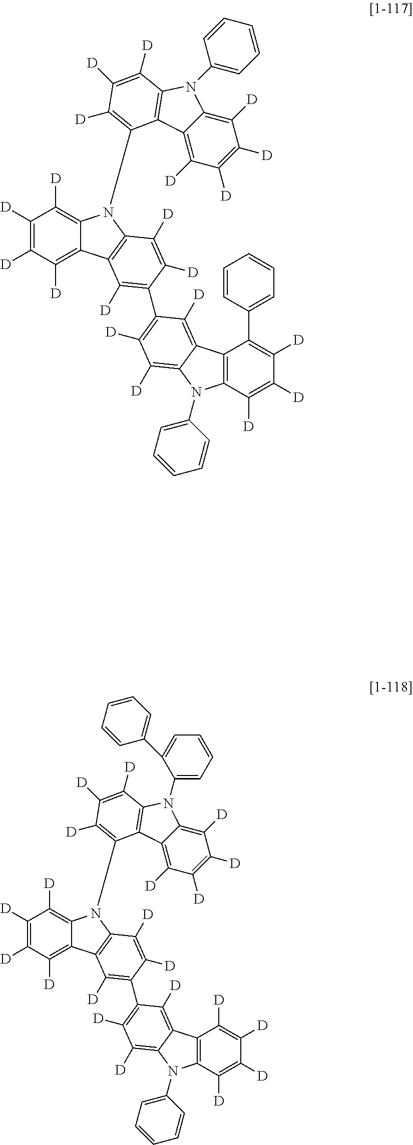 [SMILES: [2H]c1c([2H])c([2H])c2c(c1[2H])c1c(-n3c4c([2H])c([2H])c([2H])c([2H])c4c4c([2H])c(-c5c([2H])c([2H])c6c(c5[2H])c5c(-c7ccccc7)c([2H])c([2H])c([2H])c5n6-c5ccccc5)c([2H])c([2H])c43)c([2H])c([2H])c([2H])c1n2-c1ccccc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c([2H])c4c3c3c([2H])c([2H])c([2H])c([2H])c3n4-c3ccccc3-c3ccccc3)c([2H])c([2H])c1n2-c1ccccc1]

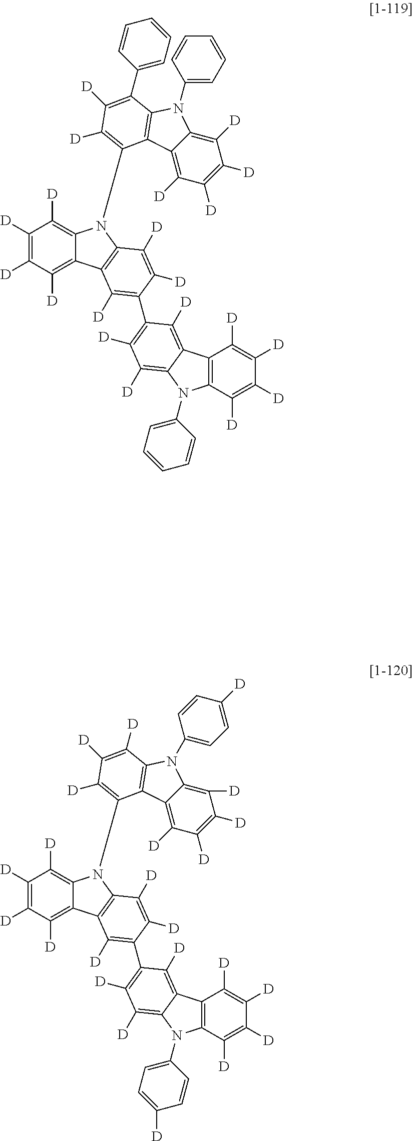 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c(-c4ccccc4)c4c3c3c([2H])c([2H])c([2H])c([2H])c3n4-c3ccccc3)c([2H])c([2H])c1n2-c1ccccc1 ; [2H]c1ccc(-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c4c4c([2H])c([2H])c([2H])c([2H])c4n5-c4ccc([2H])cc4)c([2H])c([2H])c32)cc1]

A composition for an organic optoelectronic device according to another embodiment may include, e.g., a first compound and a second compound. In an implementation, the first compound may be, e.g., the aforementioned compound for an organic optoelectronic device, and the second compound may be, e.g., a compound for an optoelectronic device represented by Chemical Formula 2.

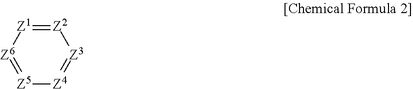

In Chemical Formula 2,

Z¹ may be, e.g., N or C-L³-R¹⁹,

Z² may be, e.g., N or C-L⁴-R²⁰,

Z³ may be, e.g., N or C-L⁵-R²¹,

Z⁴ may be, e.g., N or C-L⁶-R²²,

Z⁵ may be, e.g., N or C-L⁷-R²³, and

Z⁶ may be, e.g., N or C-L⁸-R²⁴.

In an implementation, at least two of Z¹ to Z⁶ are N.

L³ to L⁸ may each independently be or include, e.g., a single bond, a substituted or unsubstituted C6 to C20 arylene group, a substituted or unsubstituted C2 to C20 heterocyclic group, or a combination thereof.

R¹⁹ to R²⁴ may each independently be or include, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof.

In an implementation, R¹⁹ to R²⁴ may each be separately present or adjacent ones thereof may be linked to each other to provide, e.g., a substituted or unsubstituted aliphatic monocyclic ring, a substituted or unsubstituted aliphatic polycyclic ring, a substituted or unsubstituted aromatic monocyclic ring, a substituted or unsubstituted to form an aromatic polycyclic ring, a substituted or unsubstituted heteroaromatic monocyclic ring, or a substituted or unsubstituted heteroaromatic polycyclic ring.

In an implementation, when R¹⁹ to R²⁴ are separately present, at least one of R¹⁹ to R²⁴ may be, e.g., a substituted or unsubstituted C10 to C30 aryl group, or a substituted or unsubstituted C2 to C30 heterocyclic group.

In an implementation, Chemical Formula 2 may be, e.g., represented by one of Chemical Formula 2-I to Chemical Formula 2-IV, depending on whether adjacent groups of R¹⁹ to R²⁴ are further fused.

In an implementation, when R¹⁹ to R²⁴ are separately present, the second compound may be represented by Chemical Formula 2-I. In an implementation, at least one of R²⁰, R²², and R²⁴ may be, e.g., a substituted or unsubstituted C10 to C30 aryl group or a substituted or unsubstituted C2 to C30 heterocyclic group.

In an implementation, R²⁰ and R²¹ may be linked to form, e.g., a substituted or unsubstituted aromatic monocyclic ring or a substituted or unsubstituted to form an aromatic polycyclic ring, and the second compound may be represented by Chemical Formula 2-II or Chemical Formula 2-III.

In an implementation, R²⁰ and R²¹ may be linked to form, e.g., a substituted or unsubstituted heteroaromatic polycyclic ring, and the second compound may be represented by Chemical Formula 2-IV.

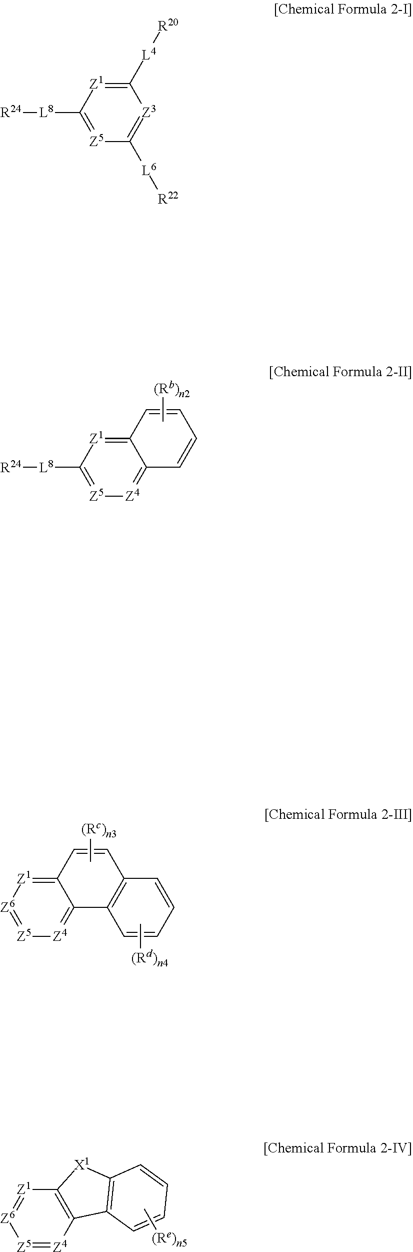

In Chemical Formula 2-I to Chemical Formula 2-IV, Z¹, Z³ to Z⁶, L⁴, L⁶, L⁸, R²⁰, R²², and R²⁴ may be defined the same as those described above.

X¹ may be, e.g., O or S.

In an implementation, at least two of Z¹, Z³, and Z⁵ of Chemical Formula 2-I are N, at least two of Z¹, Z⁴, and Z⁵ of Chemical Formula 2-II are N, and at least two of Z¹, and Z⁴ to Z⁶ of Chemical Formula 2-III and Chemical Formula 2-IV are N.

R^{b} to R^{e} may each independently be, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof.

n2, n4, and n5 may each independently be, e.g., an integer of 1 to 4.

n3 may be, e.g., 1 or 2.

In an implementation, the second compound may be represented by Chemical Formula 2-I.

In an implementation, in Chemical Formula 2-I, Z¹, Z³, and Z⁵ may each independently be N or CH, and at least two of Z¹, Z³, and Z⁵ may be N.

In an implementation, Z¹, Z³, and Z⁵ may each be N.

In an implementation, Z¹ and Z³ may be N, and Z⁵ may be CH.

In Chemical Formula 2-I, L⁴, L⁶, and L⁸ may each independently be, e.g., a single bond, a phenylene group, a biphenylene group, a carbazolylene group, a dibenzofuranylene group, a dibenzothiophenylene group, or a pyridinylene group.

In an implementation, L⁴, L⁶, and L⁸ may each independently be, e.g., a single bond, an m-phenylene group, or a p-phenylene group.

In Chemical Formula 2-I, R²⁰, R²², and R²⁴ may each independently be, e.g., a substituted or unsubstituted C6 to C30 aryl group or a substituted or unsubstituted C2 to C30 heterocyclic group, and at least one of R²⁰, R²², and R²⁴ may be a substituted or unsubstituted C10 to C30 aryl group, or a substituted or unsubstituted C2 to C30 heterocyclic group.

In an implementation, R²⁰, R²², and R²⁴ may each independently be, e.g., a substituted or unsubstituted phenyl group, a substituted or unsubstituted biphenyl group, a substituted or unsubstituted terphenyl group, a substituted or unsubstituted naphthyl group, a substituted or unsubstituted triphenylene group, a substituted or unsubstituted carbazolyl group, a substituted or unsubstituted indolocarbazolyl group, a substituted or unsubstituted dibenzofuranyl group, a substituted or unsubstituted dibenzothiophenyl group, a substituted or unsubstituted fused carbazolyl group, a substituted or unsubstituted fused dibenzofuranyl group, a substituted or unsubstituted fused dibenzothiophenyl group, a substituted or unsubstituted fused indolocarbazolyl group, a substituted or unsubstituted pyridinyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted quinoxalinyl group, a substituted or unsubstituted quinazolinyl group, or a substituted or unsubstituted benzoquinazolinyl group.

In an implementation, at least one of R²⁰, R²², and R²⁴ may be, e.g., a substituted or unsubstituted triphenylene group, a substituted or unsubstituted carbazolyl group, a substituted or unsubstituted indolocarbazolyl group, a substituted or unsubstituted dibenzofuranyl group, a substituted or unsubstituted dibenzothiophenyl group, a substituted or unsubstituted fused carbazolyl group, a substituted or unsubstituted fused dibenzofuranyl group, a substituted or unsubstituted fused dibenzothiophenyl group, a substituted or unsubstituted fused indolocarbazolyl group, a substituted or unsubstituted pyridinyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted quinoxalinyl group, a substituted or unsubstituted quinazolinyl group, or a substituted or unsubstituted benzoquinazolinyl group.

In an implementation, R²⁰, R²², and R²⁴ may each independently be, e.g., a substituted or unsubstituted phenyl group, a substituted or unsubstituted biphenyl group, a substituted or unsubstituted terphenyl group, a substituted or unsubstituted triphenylene group, a substituted or unsubstituted carbazolyl group, a substituted or unsubstituted indolocarbazolyl group, a substituted or unsubstituted dibenzofuranyl group, a substituted or unsubstituted dibenzothiophenyl group, or a combination thereof.

In an implementation, at least one of R²⁰, R²², and R²⁴ may be, e.g., a substituted or unsubstituted triphenylene group, a substituted or unsubstituted carbazolyl group, a substituted or unsubstituted indolocarbazolyl group, a substituted or unsubstituted dibenzofuranyl group, or a substituted or unsubstituted dibenzothiophenyl group.

In an implementation, Chemical Formula 2-I may be represented by, e.g., one of Chemical Formula 2-IA to Chemical Formula 2-ID.

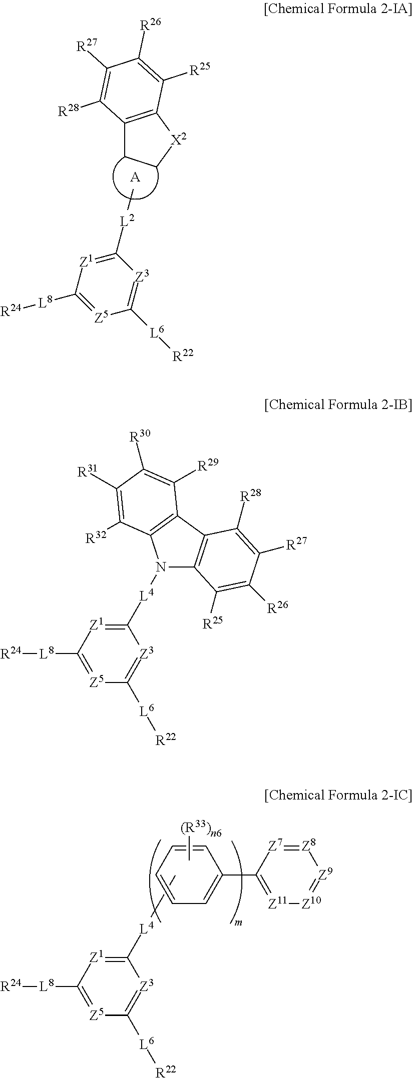

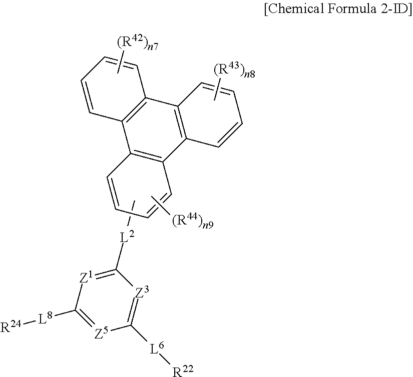

In Chemical Formula 2-IA to Chemical Formula 2-ID, Z¹, Z³, Z⁵, L⁴, L⁶, L⁸, R²², and R²⁴ may be defined the same as those described above.

X² may be, e.g., O, S, or NRⁱ.

Z⁷ may be, e.g., N or C-L⁹-R³⁴.

Z⁸ may be, e.g., N or C-L¹⁰-R³⁵.

Z⁹ may be, e.g., N or C-L¹¹-R³⁶.

Z¹⁰ may be, e.g., N or C-L¹²-R³⁷.

Z¹¹ may be, e.g., N or C-L¹³-R³⁸.

In an implementation, at least one of Z⁷ to Z¹¹ is N.

L⁹ to L¹³ may each independently be, e.g., a single bond, a substituted or unsubstituted C6 to C20 arylene group, a substituted or unsubstituted C2 to C20 heterocyclic group, or a combination thereof.

Rⁱ may be, e.g., a substituted or unsubstituted C6 to C30 aryl group, a substituted or unsubstituted C2 to C30 heterocyclic group, or a combination thereof.

R²⁵ to R³⁸ and R⁴² to R⁴⁴ may each independently be, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof.

R²⁵ to R³⁸ may each be separately present or adjacent ones thereof may be linked to each other to form, e.g., a substituted or unsubstituted aliphatic monocyclic ring, a substituted or unsubstituted aliphatic polycyclic ring, a substituted or unsubstituted aromatic monocyclic ring, a substituted or unsubstituted to form an aromatic polycyclic ring, a substituted or unsubstituted heteroaromatic monocyclic ring, or a substituted or unsubstituted heteroaromatic polycyclic ring.

m may be, e.g., an integer of 0 to 3.

n6 to n8 may each independently be, e.g., an integer of 1 to 4.

n7 may be, e.g., an integer of 1 to 3.

Ring A may be a ring represented by one of, e.g., Chemical Formula A-1 to A-6,

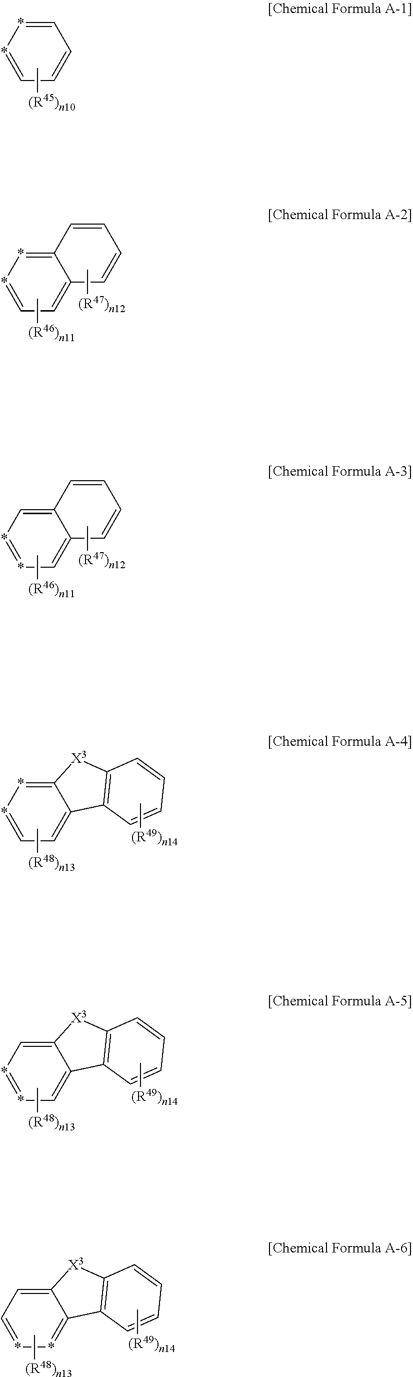

In Chemical Formula A-1 to Chemical Formula A-3, X³ may each independently be, e.g., O or S.

R⁴⁵ to R⁴⁹ may each independently be, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof.

n10, n12, and n14 may each independently be, e.g., an integer of 1 to 4.

n11 and n13 may each independently be, e.g., 1 or 2.

Each * is each fusion point, e.g., a linking carbon shared by fused rings.

In an implementation, L⁹ to L¹³ may each independently be, e.g., a single bond, a phenylene group, or a biphenylene group.

Rⁱ may be, e.g., a C6 to C12 aryl group.

m may be, e.g., an integer of 0 to 2.

In an implementation, the second compound may be, e.g., represented by Chemical Formula 2-IB.

In an implementation, adjacent groups of R²⁵ to R²⁸ may be linked to form a substituted or unsubstituted heteroaromatic polycyclic ring, and the second compound may be represented by, e.g., one of Chemical Formula 2-IB-1 to Chemical Formula 2-IB-6.

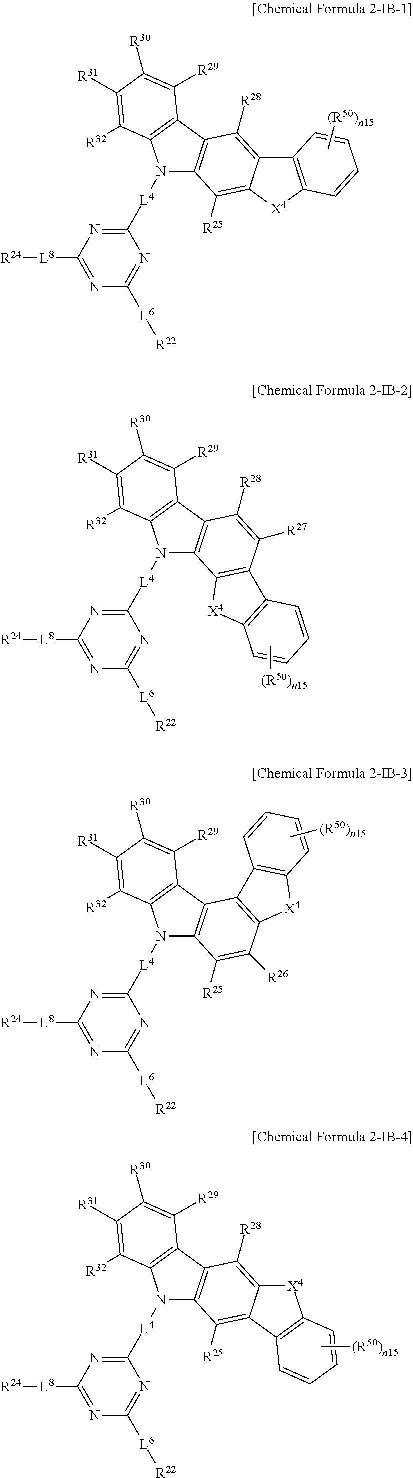

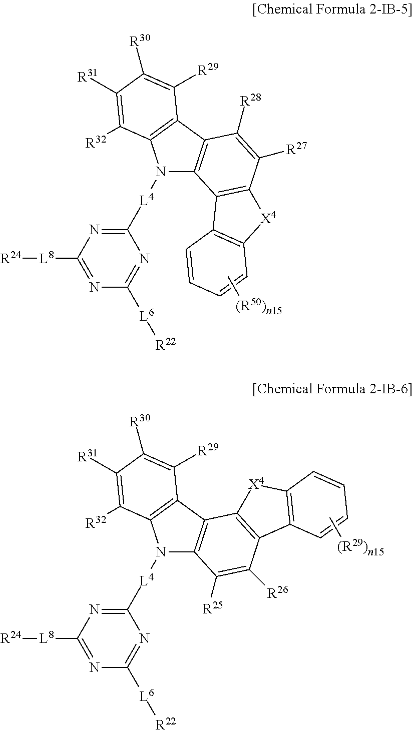

In Chemical Formula 2-IB-1 to Chemical Formula 2-IB-6, X⁴ may be, e.g., O, S CR^{j}R^{k}, or N-L¹⁴-Ar³.

L⁴, L⁶, L⁸, and L¹⁴ may each independently be, e.g., a single bond, a substituted or unsubstituted C6 to C20 arylene group, a substituted or unsubstituted C2 to C20 heterocyclic group, or a combination thereof.

R^{j} and R^{k} may each independently be, e.g., a substituted or unsubstituted C1 to C20 alkyl group, a substituted or unsubstituted C6 to C30 aryl group, or a combination thereof.

Ar³, R²², and R²⁴ may each independently be, e.g., a substituted or unsubstituted C6 to C30 aryl group, a substituted or unsubstituted C2 to C30 heterocyclic group, or a combination thereof.

R²⁵ to R³² and R⁵⁰ may each independently be, e.g., hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof.

n15 may be, e.g., an integer of 1 to 4.

In an implementation, R^{j} and R^{k} may each independently be, e.g., a substituted or unsubstituted C1 to C10 alkyl group, a substituted or unsubstituted C6 to C12 aryl group.

In an implementation, Ar³ may be, e.g., a substituted or unsubstituted C6 to C18 aryl group or a substituted or unsubstituted C2 to C20 heterocyclic group.

In an implementation, R²² and R²⁴ may each independently be, e.g., a substituted or unsubstituted C6 to C12 aryl group.

In an implementation, R²⁵ to R³² and R⁵⁰ may each independently be, e.g., hydrogen, deuterium, a cyano group, a C1 to C10 alkyl group, or a C6 to C12 aryl group.

In an implementation, R^{j} and R^{k} may each independently be, e.g., a substituted or unsubstituted methyl group, a substituted or unsubstituted ethyl group, a substituted or unsubstituted propyl group, a substituted or unsubstituted phenyl group, a substituted or unsubstituted biphenyl group, a substituted or unsubstituted naphthyl group, or a combination thereof.

In an implementation, Ar³ may be, e.g., a substituted or unsubstituted phenyl group, a substituted or unsubstituted biphenyl group, a substituted or unsubstituted terphenyl group, a substituted or unsubstituted carbazolyl group, a substituted or unsubstituted dibenzofuranyl group, or a substituted or unsubstituted dibenzothiophenyl group.

In an implementation, R²² and R²⁴ may each independently be, e.g., a substituted or unsubstituted phenyl group or a substituted or unsubstituted biphenyl group.

In an implementation, R²⁵ to R³² and R⁵⁰ may each independently be, e.g., hydrogen, deuterium, or a substituted or unsubstituted phenyl group.

In an implementation, R⁴² to R⁴⁴ may each independently be, e.g., hydrogen, deuterium, or a substituted or unsubstituted phenyl group.

In an implementation, the second compound may be represented by the aforementioned Chemical Formula 2-IB-2, in which X⁴ may be, e.g., N-L¹⁴-Ar³, in which n15 may be, e.g., an integer of 1 to 4, wherein L⁴, L⁶, L⁸, and L¹⁴ may each independently be, e.g., a single bond, or a substituted or unsubstituted phenylene group, Ar³ may be, e.g., a substituted or unsubstituted C6 to C12 aryl group, R²² and R²⁴ may each independently be, e.g., a substituted or unsubstituted phenyl group, or a substituted or unsubstituted biphenyl group, and R²⁵ to R³² and R⁵⁰ may each independently be, e.g., hydrogen or deuterium or at least one thereof may be a phenyl group.

In an implementation, the second compound represented by Chemical Formula 2 may include, e.g., a compound of Group 2.

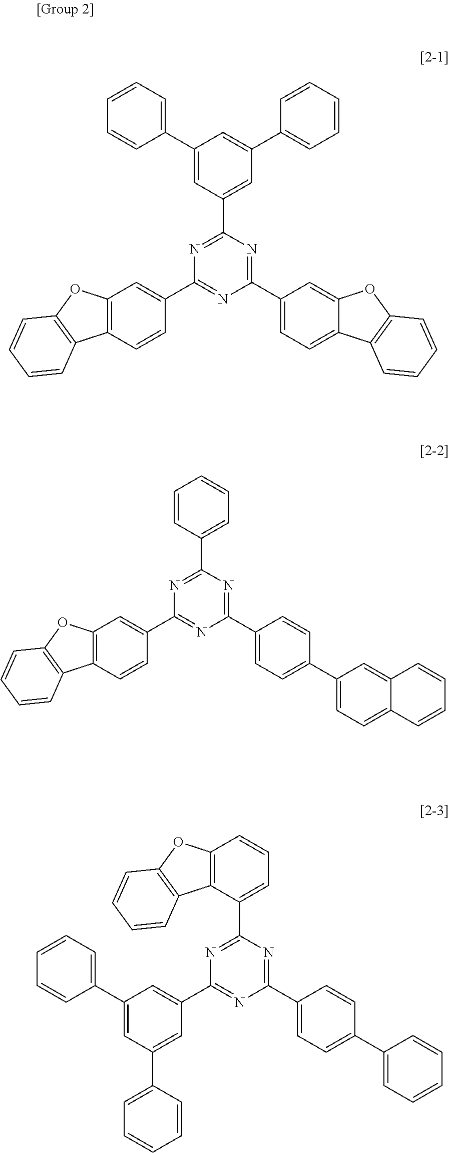 [SMILES: c1ccc(-c2cc(-c3ccccc3)cc(-c3nc(-c4ccc5c(c4)oc4ccccc45)nc(-c4ccc5c(c4)oc4ccccc45)n3)c2)cc1 ; c1ccc(-c2nc(-c3ccc(-c4ccc5ccccc5c4)cc3)nc(-c3ccc4c(c3)oc3ccccc34)n2)cc1 ; c1ccc(-c2ccc(-c3nc(-c4cc(-c5ccccc5)cc(-c5ccccc5)c4)nc(-c4cccc5oc6ccccc6c45)n3)cc2)cc1]

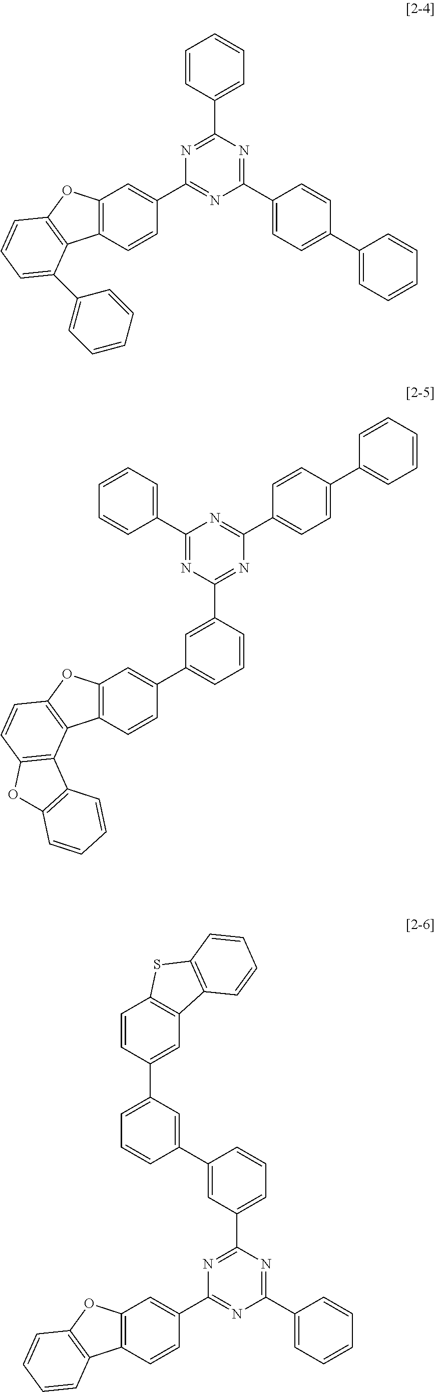 [SMILES: c1ccc(-c2ccc(-c3nc(-c4ccccc4)nc(-c4ccc5c(c4)oc4cccc(-c6ccccc6)c45)n3)cc2)cc1 ; c1ccc(-c2ccc(-c3nc(-c4ccccc4)nc(-c4cccc(-c5ccc6c(c5)oc5ccc7oc8ccccc8c7c56)c4)n3)cc2)cc1 ; c1ccc(-c2nc(-c3cccc(-c4cccc(-c5ccc6sc7ccccc7c6c5)c4)c3)nc(-c3ccc4c(c3)oc3ccccc34)n2)cc1]

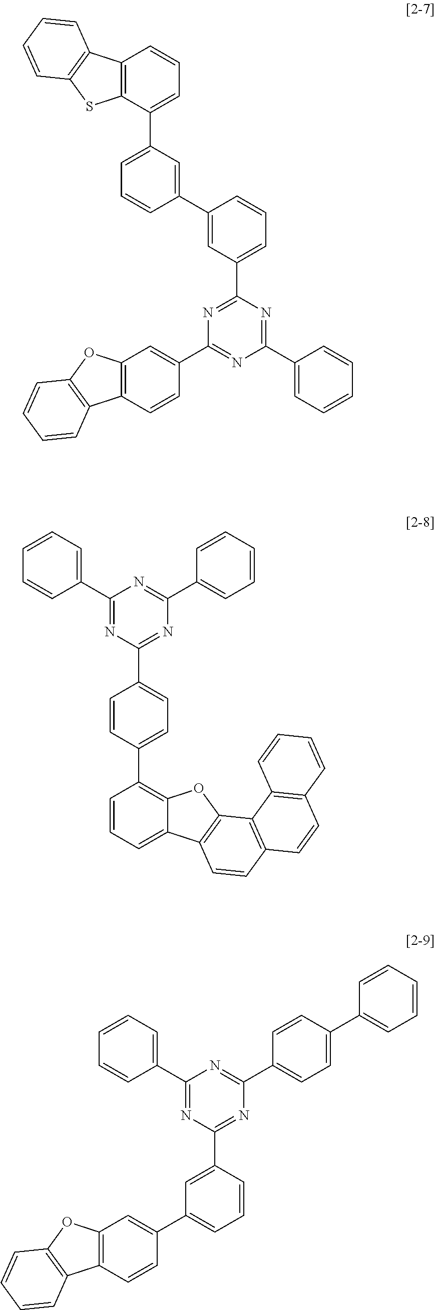 [SMILES: c1ccc(-c2nc(-c3cccc(-c4cccc(-c5cccc6c5sc5ccccc56)c4)c3)nc(-c3ccc4c(c3)oc3ccccc34)n2)cc1 ; c1ccc(-c2nc(-c3ccccc3)nc(-c3ccc(-c4cccc5c4oc4c5ccc5ccc6ccccc6c54)cc3)n2)cc1 ; c1ccc(-c2ccc(-c3nc(-c4ccccc4)nc(-c4cccc(-c5ccc6c(c5)oc5ccccc56)c4)n3)cc2)cc1]

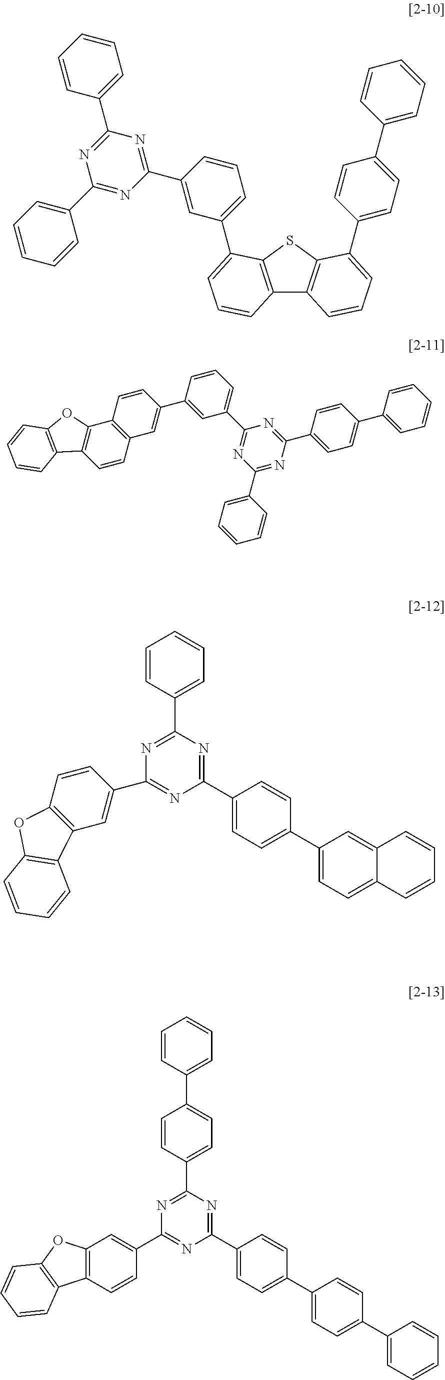 [SMILES: c1ccc(-c2ccc(-c3cccc4c3sc3c(-c5cccc(-c6nc(-c7ccccc7)nc(-c7ccccc7)n6)c5)cccc34)cc2)cc1 ; c1ccc(-c2ccc(-c3nc(-c4ccccc4)nc(-c4cccc(-c5ccc6c(ccc7c8ccccc8oc67)c5)c4)n3)cc2)cc1 ; c1ccc(-c2nc(-c3ccc(-c4ccc5ccccc5c4)cc3)nc(-c3ccc4oc5ccccc5c4c3)n2)cc1 ; c1ccc(-c2ccc(-c3ccc(-c4nc(-c5ccc(-c6ccccc6)cc5)nc(-c5ccc6c(c5)oc5ccccc56)n4)cc3)cc2)cc1]

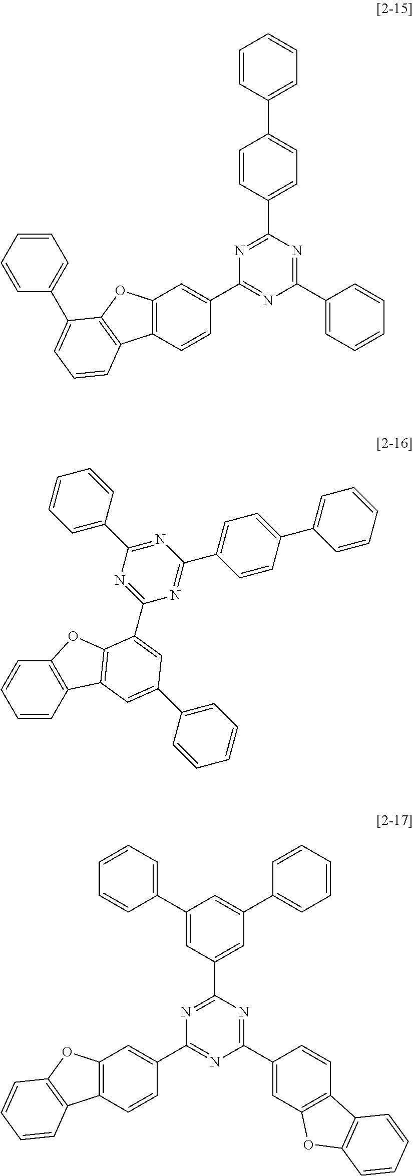 [SMILES: c1ccc(-c2ccc(-c3nc(-c4ccccc4)nc(-c4ccc5c(c4)oc4c(-c6ccccc6)cccc45)n3)cc2)cc1 ; c1ccc(-c2ccc(-c3nc(-c4ccccc4)nc(-c4cc(-c5ccccc5)cc5c4oc4ccccc45)n3)cc2)cc1 ; c1ccc(-c2cc(-c3ccccc3)cc(-c3nc(-c4ccc5c(c4)oc4ccccc45)nc(-c4ccc5c(c4)oc4ccccc45)n3)c2)cc1]

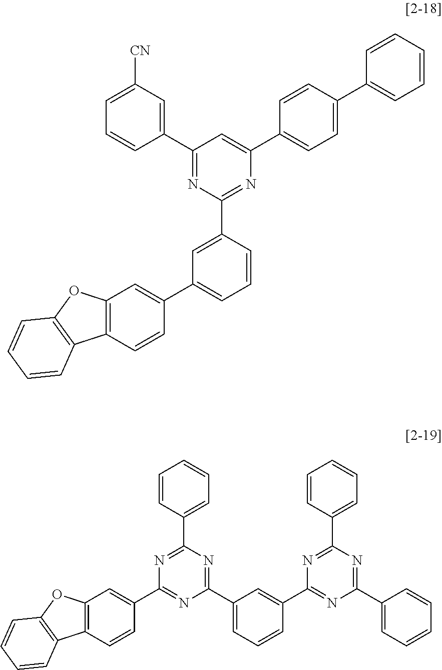 [SMILES: N#Cc1cccc(-c2cc(-c3ccc(-c4ccccc4)cc3)nc(-c3cccc(-c4ccc5c(c4)oc4ccccc45)c3)n2)c1 ; c1ccc(-c2nc(-c3ccccc3)nc(-c3cccc(-c4nc(-c5ccccc5)nc(-c5ccc6c(c5)oc5ccccc56)n4)c3)n2)cc1]

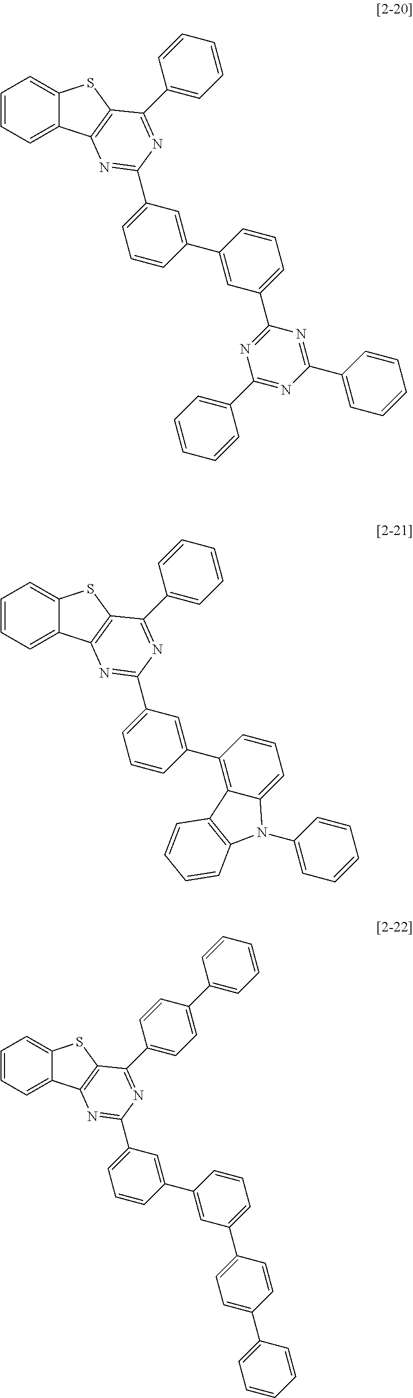 [SMILES: c1ccc(-c2nc(-c3ccccc3)nc(-c3cccc(-c4cccc(-c5nc(-c6ccccc6)c6sc7ccccc7c6n5)c4)c3)n2)cc1 ; c1ccc(-c2nc(-c3cccc(-c4cccc5c4c4ccccc4n5-c4ccccc4)c3)nc3c2sc2ccccc23)cc1 ; c1ccc(-c2ccc(-c3cccc(-c4cccc(-c5nc(-c6ccc(-c7ccccc7)cc6)c6sc7ccccc7c6n5)c4)c3)cc2)cc1]

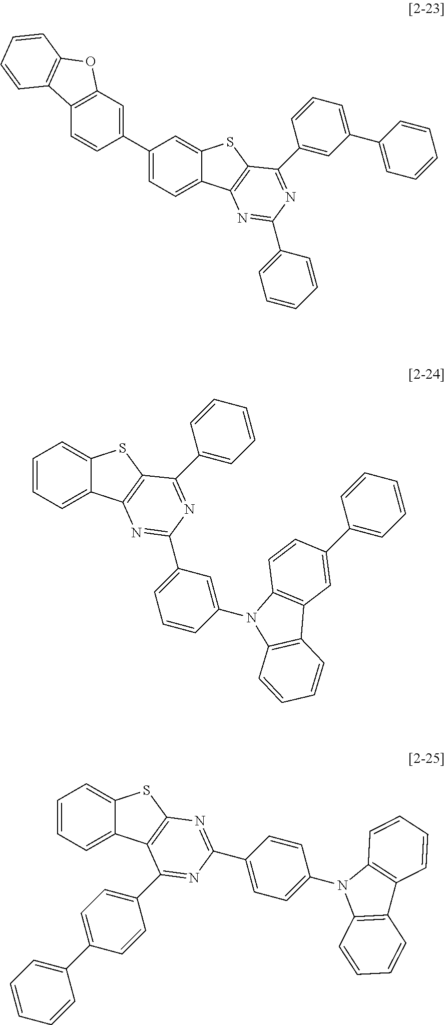 [SMILES: c1ccc(-c2cccc(-c3nc(-c4ccccc4)nc4c3sc3cc(-c5ccc6c(c5)oc5ccccc56)ccc34)c2)cc1 ; c1ccc(-c2ccc3c(c2)c2ccccc2n3-c2cccc(-c3nc(-c4ccccc4)c4sc5ccccc5c4n3)c2)cc1 ; c1ccc(-c2ccc(-c3nc(-c4ccc(-n5c6ccccc6c6ccccc65)cc4)nc4sc5ccccc5c34)cc2)cc1]

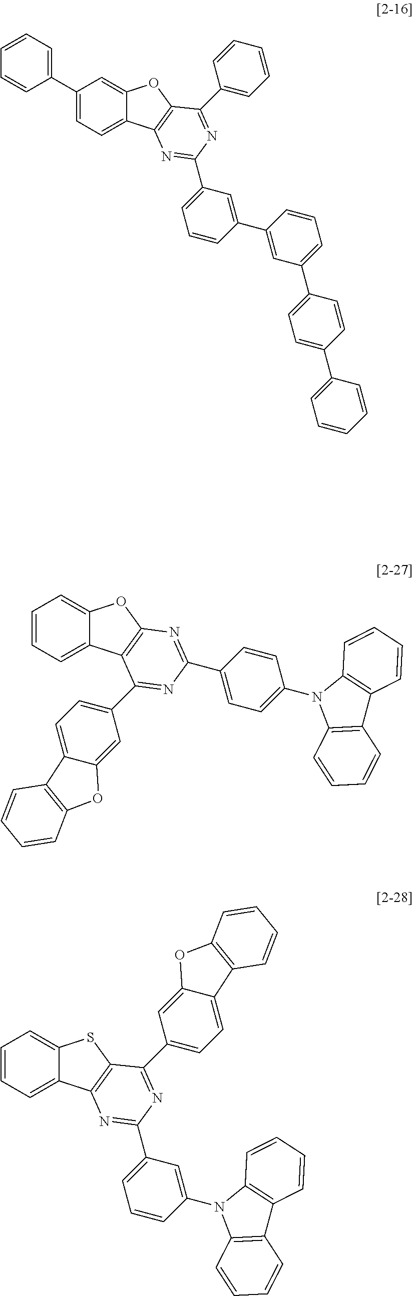 [SMILES: c1ccc(-c2ccc(-c3cccc(-c4cccc(-c5nc(-c6ccccc6)c6oc7cc(-c8ccccc8)ccc7c6n5)c4)c3)cc2)cc1 ; c1ccc2c(c1)oc1cc(-c3nc(-c4ccc(-n5c6ccccc6c6ccccc65)cc4)nc4oc5ccccc5c34)ccc12 ; c1cc(-c2nc(-c3ccc4c(c3)oc3ccccc34)c3sc4ccccc4c3n2)cc(-n2c3ccccc3c3ccccc32)c1]

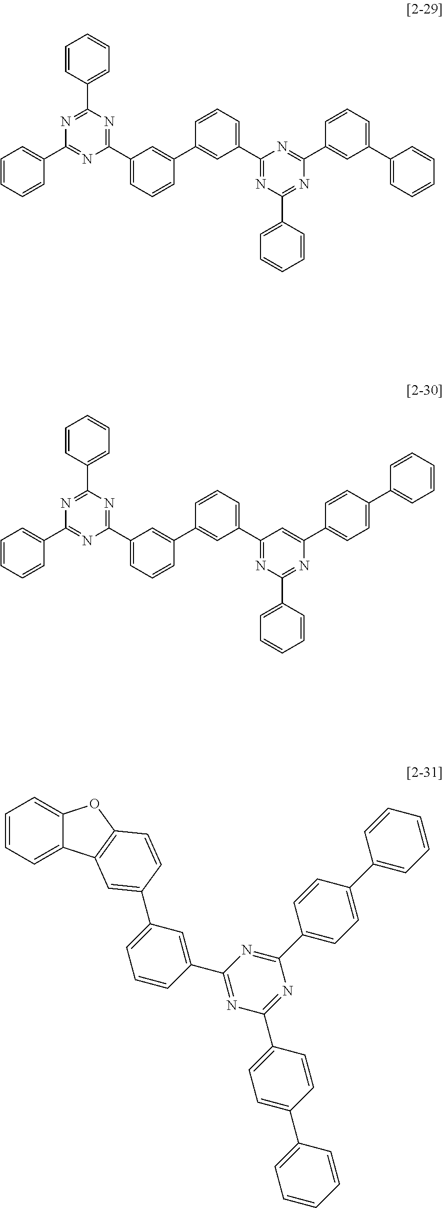 [SMILES: c1ccc(-c2cccc(-c3nc(-c4ccccc4)nc(-c4cccc(-c5cccc(-c6nc(-c7ccccc7)nc(-c7ccccc7)n6)c5)c4)n3)c2)cc1 ; c1ccc(-c2ccc(-c3cc(-c4cccc(-c5cccc(-c6nc(-c7ccccc7)nc(-c7ccccc7)n6)c5)c4)nc(-c4ccccc4)n3)cc2)cc1 ; c1ccc(-c2ccc(-c3nc(-c4ccc(-c5ccccc5)cc4)nc(-c4cccc(-c5ccc6oc7ccccc7c6c5)c4)n3)cc2)cc1]

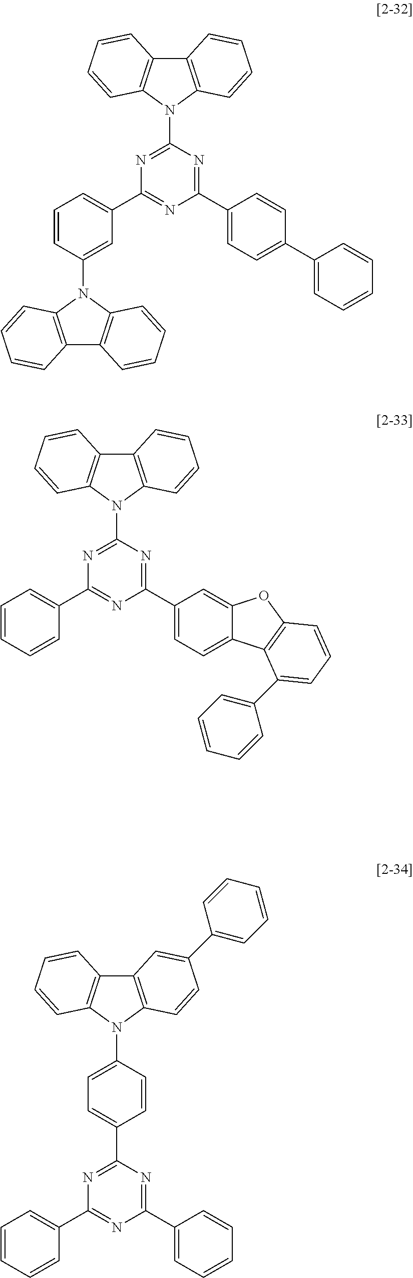 [SMILES: c1ccc(-c2ccc(-c3nc(-c4cccc(-n5c6ccccc6c6ccccc65)c4)nc(-n4c5ccccc5c5ccccc54)n3)cc2)cc1 ; c1ccc(-c2nc(-c3ccc4c(c3)oc3cccc(-c5ccccc5)c34)nc(-n3c4ccccc4c4ccccc43)n2)cc1 ; c1ccc(-c2ccc3c(c2)c2ccccc2n3-c2ccc(-c3nc(-c4ccccc4)nc(-c4ccccc4)n3)cc2)cc1]

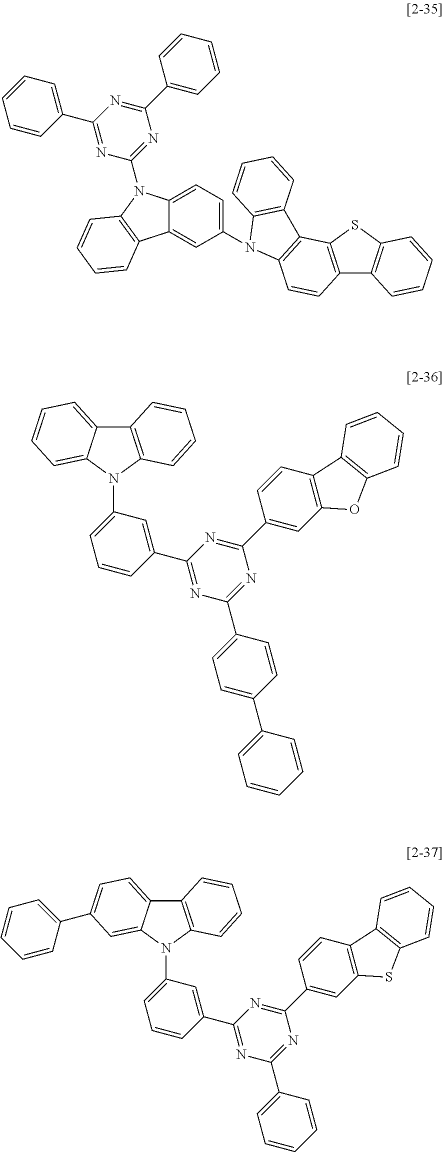 [SMILES: c1ccc(-c2nc(-c3ccccc3)nc(-n3c4ccccc4c4cc(-n5c6ccccc6c6c7sc8ccccc8c7ccc65)ccc43)n2)cc1 ; c1ccc(-c2ccc(-c3nc(-c4cccc(-n5c6ccccc6c6ccccc65)c4)nc(-c4ccc5c(c4)oc4ccccc45)n3)cc2)cc1 ; c1ccc(-c2ccc3c4ccccc4n(-c4cccc(-c5nc(-c6ccccc6)nc(-c6ccc7c(c6)sc6ccccc67)n5)c4)c3c2)cc1]

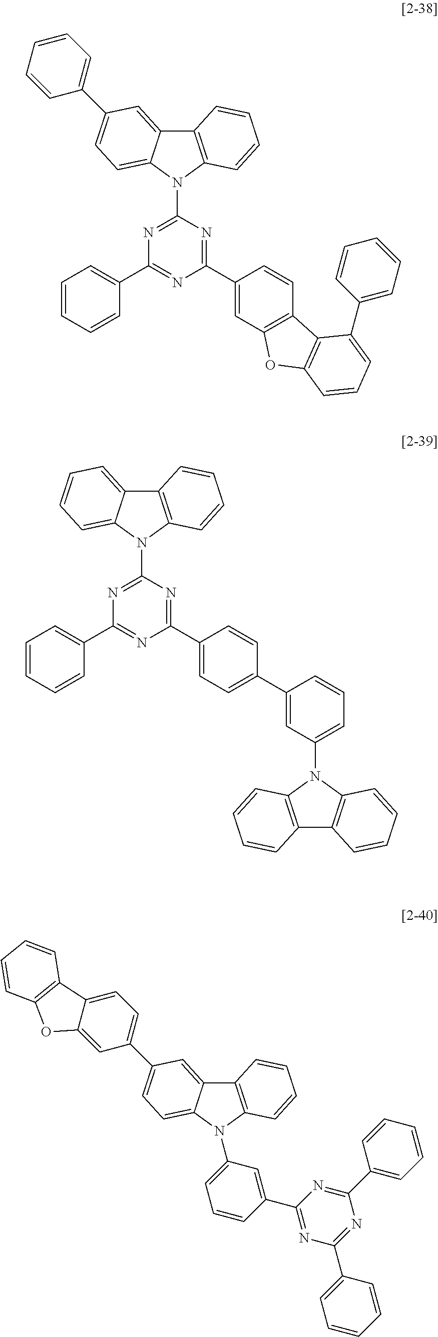 [SMILES: c1ccc(-c2ccc3c(c2)c2ccccc2n3-c2nc(-c3ccccc3)nc(-c3ccc4c(c3)oc3cccc(-c5ccccc5)c34)n2)cc1 ; c1ccc(-c2nc(-c3ccc(-c4cccc(-n5c6ccccc6c6ccccc65)c4)cc3)nc(-n3c4ccccc4c4ccccc43)n2)cc1 ; c1ccc(-c2nc(-c3ccccc3)nc(-c3cccc(-n4c5ccccc5c5cc(-c6ccc7c(c6)oc6ccccc67)ccc54)c3)n2)cc1]

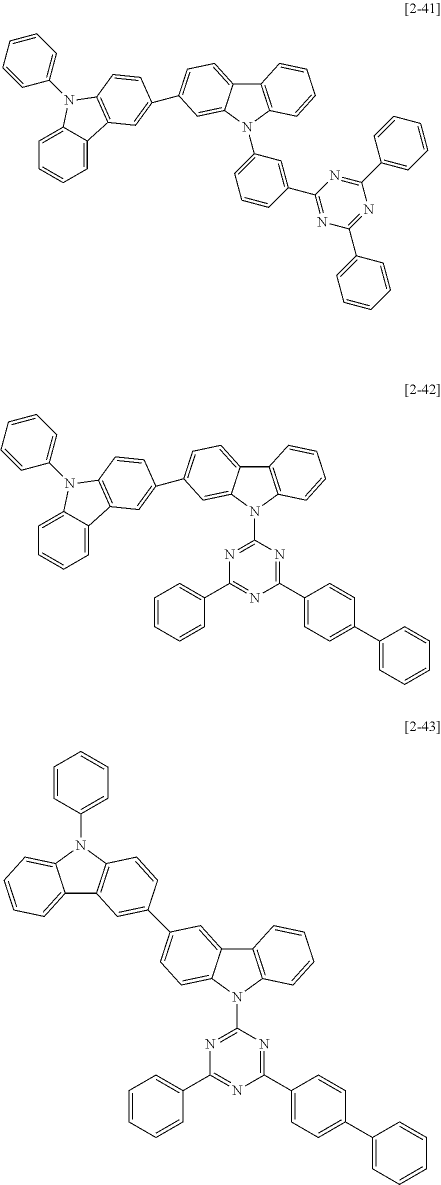 [SMILES: c1ccc(-c2nc(-c3ccccc3)nc(-c3cccc(-n4c5ccccc5c5ccc(-c6ccc7c(c6)c6ccccc6n7-c6ccccc6)cc54)c3)n2)cc1 ; c1ccc(-c2ccc(-c3nc(-c4ccccc4)nc(-n4c5ccccc5c5ccc(-c6ccc7c(c6)c6ccccc6n7-c6ccccc6)cc54)n3)cc2)cc1 ; c1ccc(-c2ccc(-c3nc(-c4ccccc4)nc(-n4c5ccccc5c5cc(-c6ccc7c(c6)c6ccccc6n7-c6ccccc6)ccc54)n3)cc2)cc1]

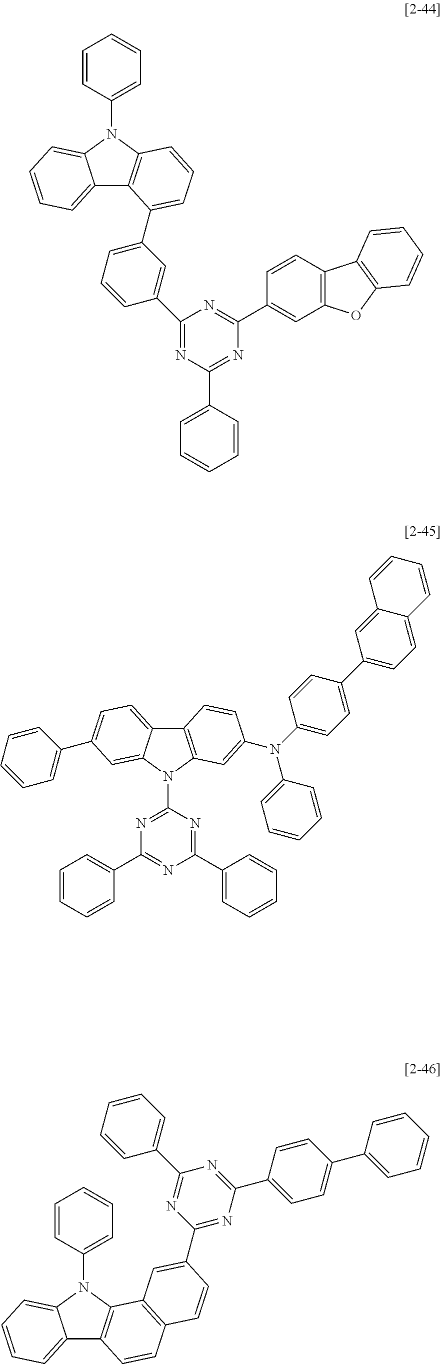 [SMILES: c1ccc(-c2nc(-c3cccc(-c4cccc5c4c4ccccc4n5-c4ccccc4)c3)nc(-c3ccc4c(c3)oc3ccccc34)n2)cc1 ; c1ccc(-c2ccc3c4ccc(N(c5ccccc5)c5ccc(-c6ccc7ccccc7c6)cc5)cc4n(-c4nc(-c5ccccc5)nc(-c5ccccc5)n4)c3c2)cc1 ; c1ccc(-c2ccc(-c3nc(-c4ccccc4)nc(-c4ccc5ccc6c7ccccc7n(-c7ccccc7)c6c5c4)n3)cc2)cc1]

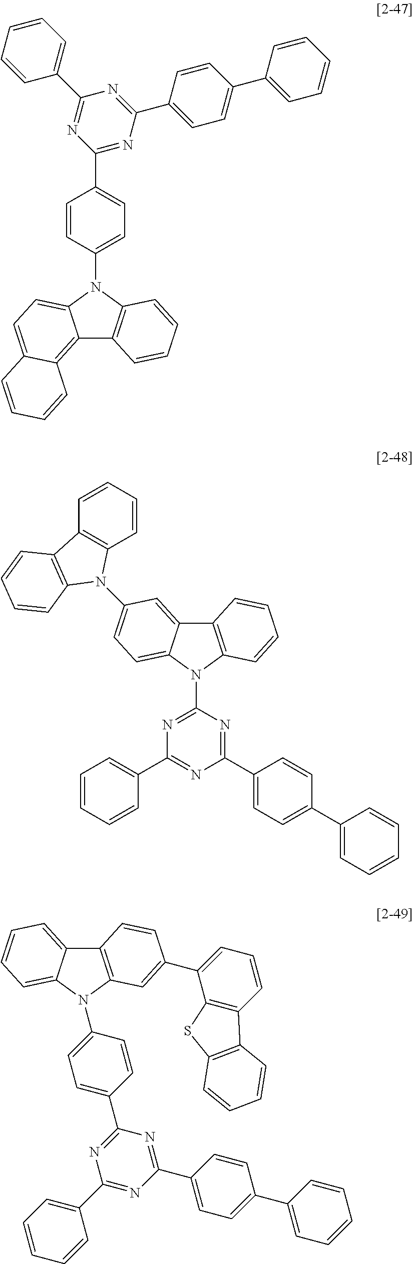 [SMILES: c1ccc(-c2ccc(-c3nc(-c4ccccc4)nc(-c4ccc(-n5c6ccccc6c6c7ccccc7ccc65)cc4)n3)cc2)cc1 ; c1ccc(-c2ccc(-c3nc(-c4ccccc4)nc(-n4c5ccccc5c5cc(-n6c7ccccc7c7ccccc76)ccc54)n3)cc2)cc1 ; c1ccc(-c2ccc(-c3nc(-c4ccccc4)nc(-c4ccc(-n5c6ccccc6c6ccc(-c7cccc8c7sc7ccccc78)cc65)cc4)n3)cc2)cc1]

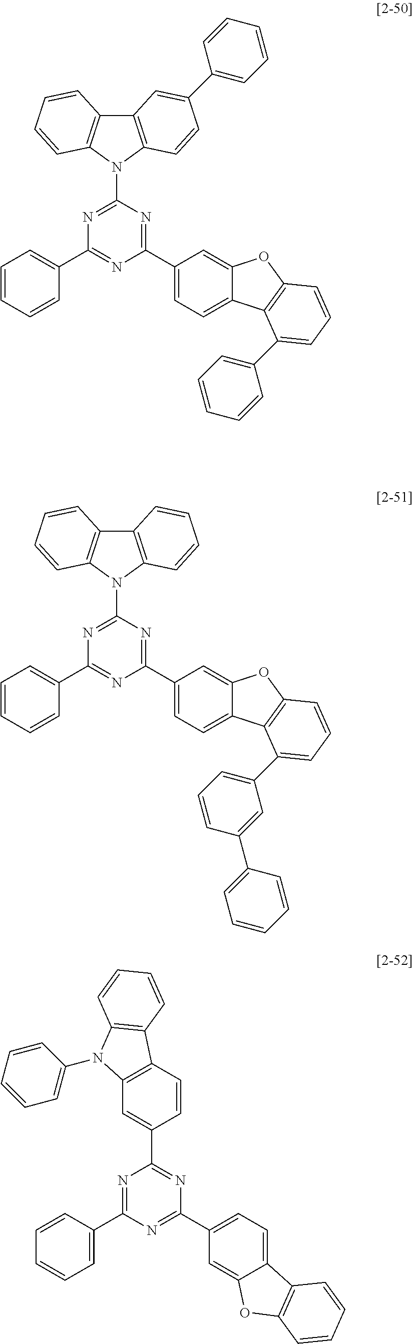 [SMILES: c1ccc(-c2ccc3c(c2)c2ccccc2n3-c2nc(-c3ccccc3)nc(-c3ccc4c(c3)oc3cccc(-c5ccccc5)c34)n2)cc1 ; c1ccc(-c2cccc(-c3cccc4oc5cc(-c6nc(-c7ccccc7)nc(-n7c8ccccc8c8ccccc87)n6)ccc5c34)c2)cc1 ; c1ccc(-c2nc(-c3ccc4c(c3)oc3ccccc34)nc(-c3ccc4c5ccccc5n(-c5ccccc5)c4c3)n2)cc1]

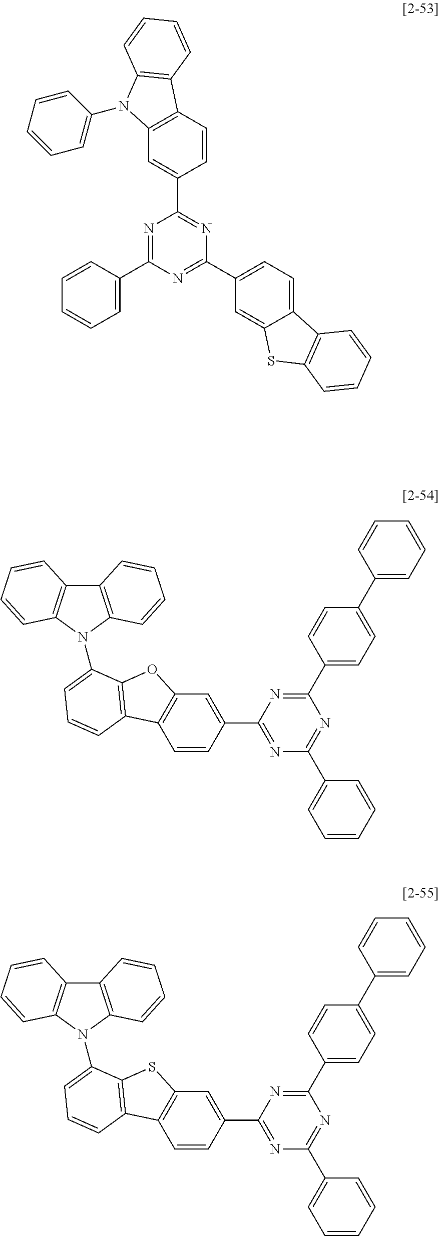 [SMILES: c1ccc(-c2nc(-c3ccc4c(c3)sc3ccccc34)nc(-c3ccc4c5ccccc5n(-c5ccccc5)c4c3)n2)cc1 ; c1ccc(-c2ccc(-c3nc(-c4ccccc4)nc(-c4ccc5c(c4)oc4c(-n6c7ccccc7c7ccccc76)cccc45)n3)cc2)cc1 ; c1ccc(-c2ccc(-c3nc(-c4ccccc4)nc(-c4ccc5c(c4)sc4c(-n6c7ccccc7c7ccccc76)cccc45)n3)cc2)cc1]

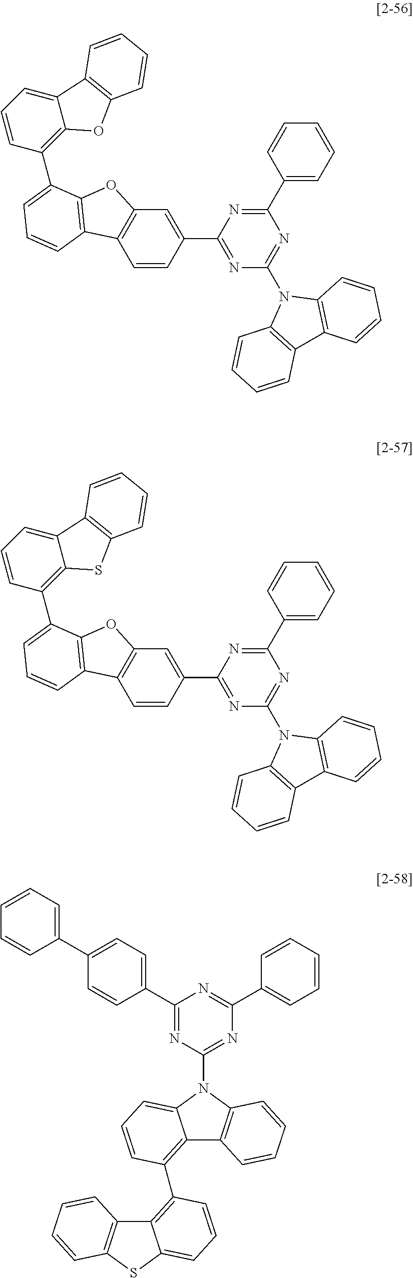 [SMILES: c1ccc(-c2nc(-c3ccc4c(c3)oc3c(-c5cccc6c5oc5ccccc56)cccc34)nc(-n3c4ccccc4c4ccccc43)n2)cc1 ; c1ccc(-c2nc(-c3ccc4c(c3)oc3c(-c5cccc6c5sc5ccccc56)cccc34)nc(-n3c4ccccc4c4ccccc43)n2)cc1 ; c1ccc(-c2ccc(-c3nc(-c4ccccc4)nc(-n4c5ccccc5c5c(-c6cccc7sc8ccccc8c67)cccc54)n3)cc2)cc1]

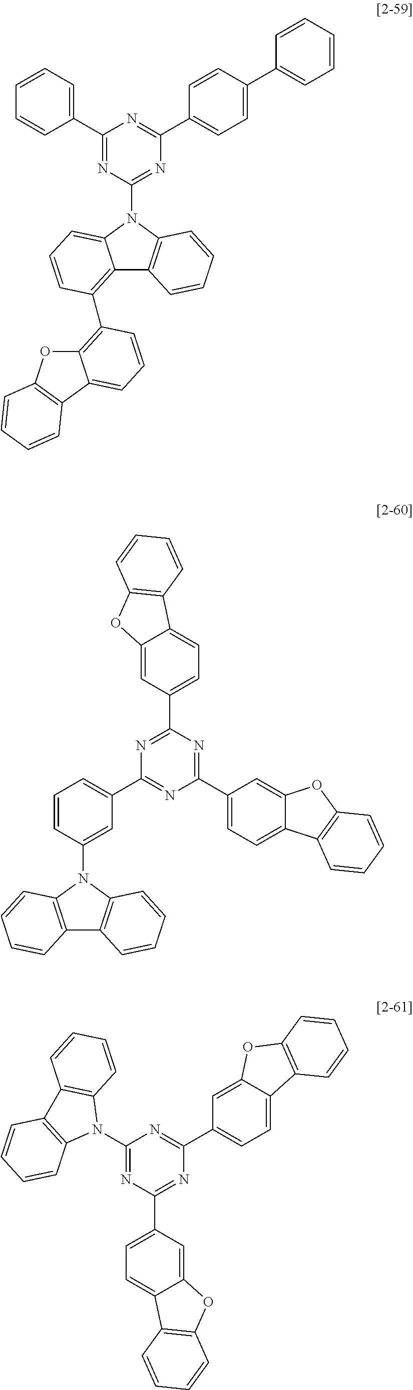 [SMILES: c1ccc(-c2ccc(-c3nc(-c4ccccc4)nc(-n4c5ccccc5c5c(-c6cccc7c6oc6ccccc67)cccc54)n3)cc2)cc1 ; c1cc(-c2nc(-c3ccc4c(c3)oc3ccccc34)nc(-c3ccc4c(c3)oc3ccccc34)n2)cc(-n2c3ccccc3c3ccccc32)c1 ; c1ccc2c(c1)oc1cc(-c3nc(-c4ccc5c(c4)oc4ccccc45)nc(-n4c5ccccc5c5ccccc54)n3)ccc12]

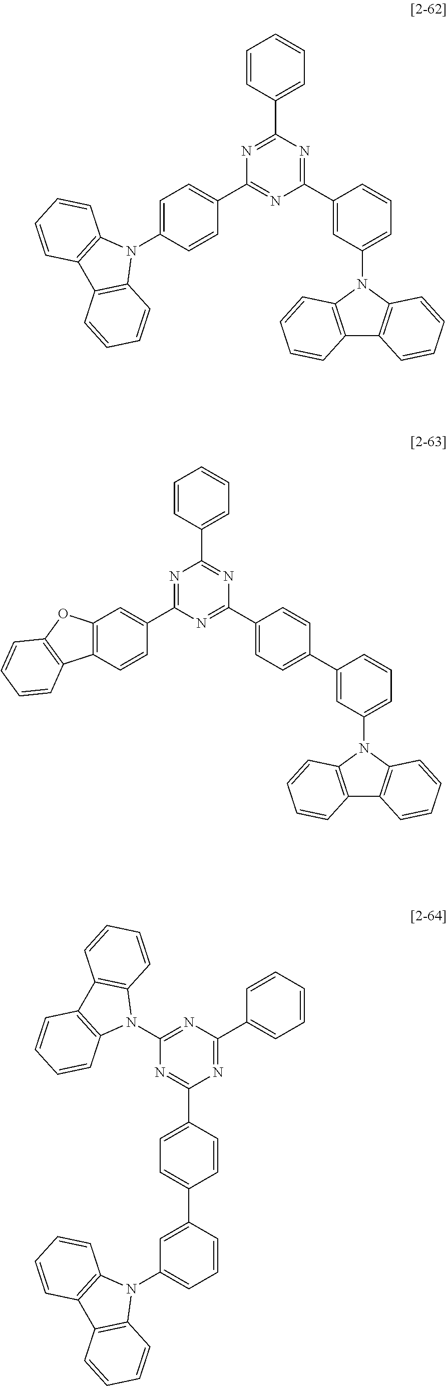 [SMILES: c1ccc(-c2nc(-c3ccc(-n4c5ccccc5c5ccccc54)cc3)nc(-c3cccc(-n4c5ccccc5c5ccccc54)c3)n2)cc1 ; c1ccc(-c2nc(-c3ccc(-c4cccc(-n5c6ccccc6c6ccccc65)c4)cc3)nc(-c3ccc4c(c3)oc3ccccc34)n2)cc1 ; c1ccc(-c2nc(-c3ccc(-c4cccc(-n5c6ccccc6c6ccccc65)c4)cc3)nc(-n3c4ccccc4c4ccccc43)n2)cc1]

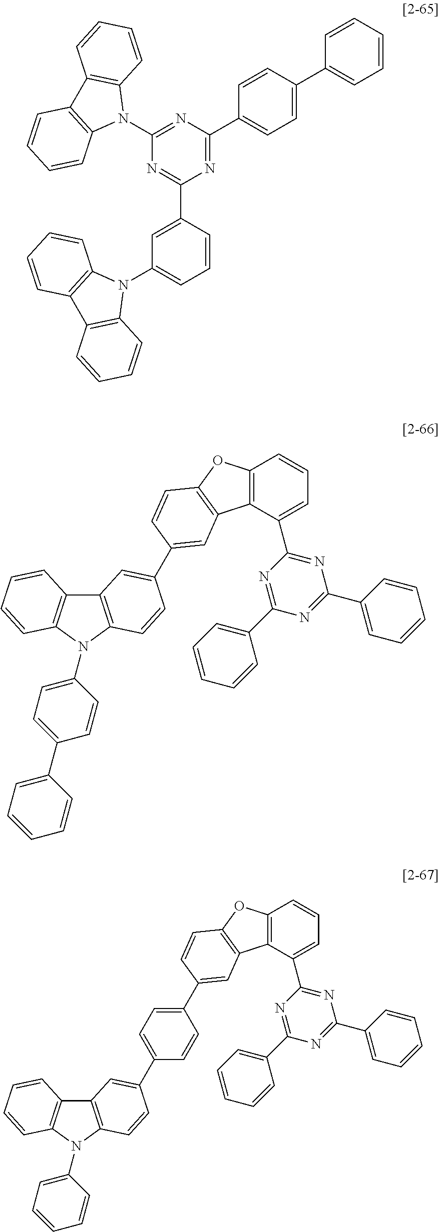 [SMILES: c1ccc(-c2ccc(-c3nc(-c4cccc(-n5c6ccccc6c6ccccc65)c4)nc(-n4c5ccccc5c5ccccc54)n3)cc2)cc1 ; c1ccc(-c2ccc(-n3c4ccccc4c4cc(-c5ccc6oc7cccc(-c8nc(-c9ccccc9)nc(-c9ccccc9)n8)c7c6c5)ccc43)cc2)cc1 ; c1ccc(-c2nc(-c3ccccc3)nc(-c3cccc4oc5ccc(-c6ccc(-c7ccc8c(c7)c7ccccc7n8-c7ccccc7)cc6)cc5c34)n2)cc1]

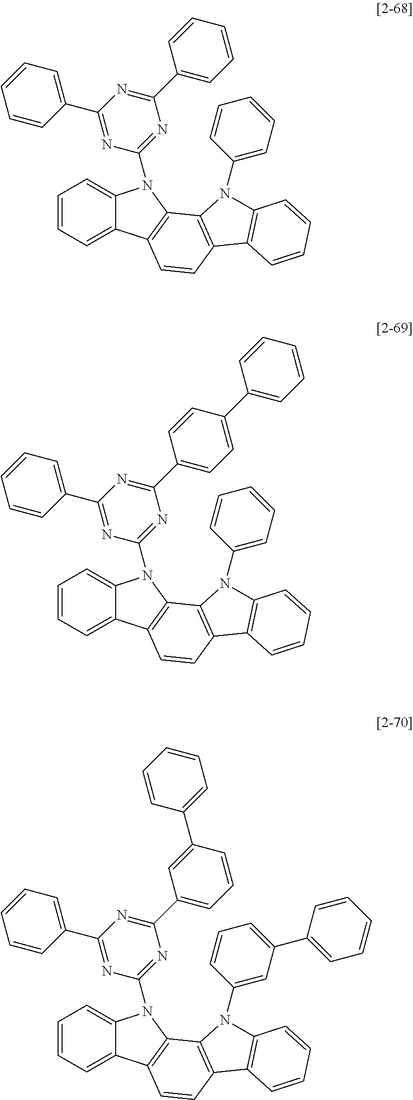 [SMILES: c1ccc(-c2nc(-c3ccccc3)nc(-n3c4ccccc4c4ccc5c6ccccc6n(-c6ccccc6)c5c43)n2)cc1 ; c1ccc(-c2ccc(-c3nc(-c4ccccc4)nc(-n4c5ccccc5c5ccc6c7ccccc7n(-c7ccccc7)c6c54)n3)cc2)cc1 ; c1ccc(-c2cccc(-c3nc(-c4ccccc4)nc(-n4c5ccccc5c5ccc6c7ccccc7n(-c7cccc(-c8ccccc8)c7)c6c54)n3)c2)cc1]

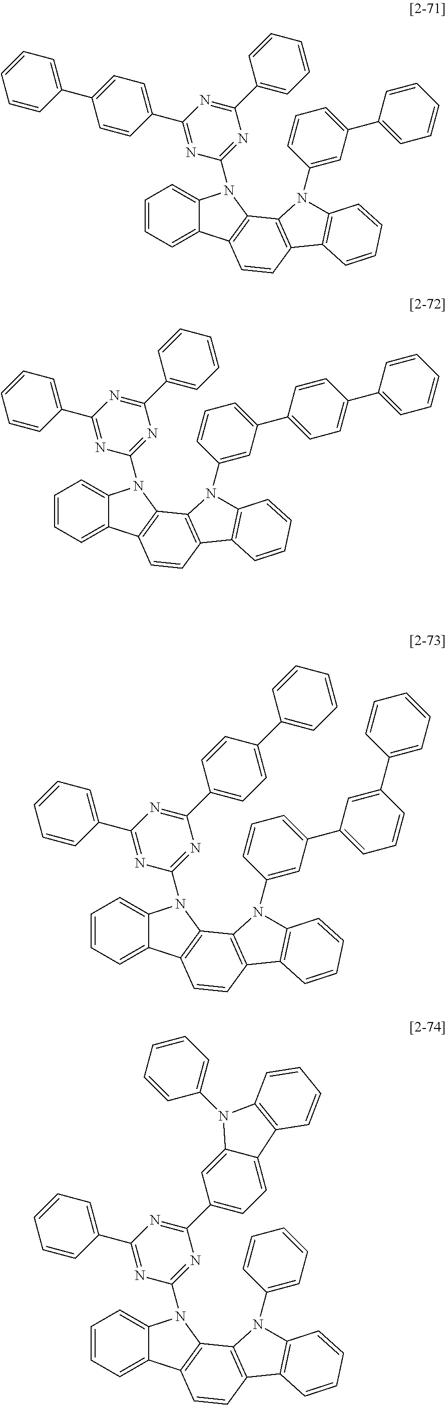 [SMILES: c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1 ; c1ccc(-c2ccc(-c3cccc(-n4c5ccccc5c5ccc6c7ccccc7n(-c7nc(-c8ccccc8)nc(-c8ccccc8)n7)c6c54)c3)cc2)cc1 ; c1ccc(-c2ccc(-c3nc(-c4ccccc4)nc(-n4c5ccccc5c5ccc6c7ccccc7n(-c7cccc(-c8cccc(-c9ccccc9)c8)c7)c6c54)n3)cc2)cc1 ; c1ccc(-c2nc(-c3ccc4c5ccccc5n(-c5ccccc5)c4c3)nc(-n3c4ccccc4c4ccc5c6ccccc6n(-c6ccccc6)c5c43)n2)cc1]

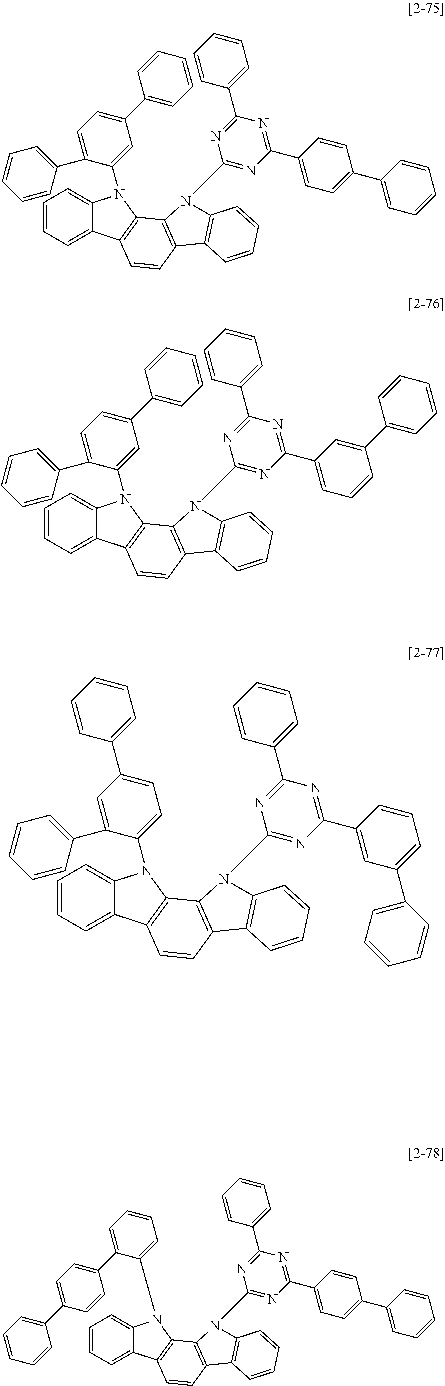 [SMILES: c1ccc(-c2ccc(-c3nc(-c4ccccc4)nc(-n4c5ccccc5c5ccc6c7ccccc7n(-c7cc(-c8ccccc8)ccc7-c7ccccc7)c6c54)n3)cc2)cc1 ; c1ccc(-c2cccc(-c3nc(-c4ccccc4)nc(-n4c5ccccc5c5ccc6c7ccccc7n(-c7cc(-c8ccccc8)ccc7-c7ccccc7)c6c54)n3)c2)cc1 ; c1ccc(-c2cccc(-c3nc(-c4ccccc4)nc(-n4c5ccccc5c5ccc6c7ccccc7n(-c7ccc(-c8ccccc8)cc7-c7ccccc7)c6c54)n3)c2)cc1 ; c1ccc(-c2ccc(-c3nc(-c4ccccc4)nc(-n4c5ccccc5c5ccc6c7ccccc7n(-c7ccccc7-c7ccc(-c8ccccc8)cc7)c6c54)n3)cc2)cc1]

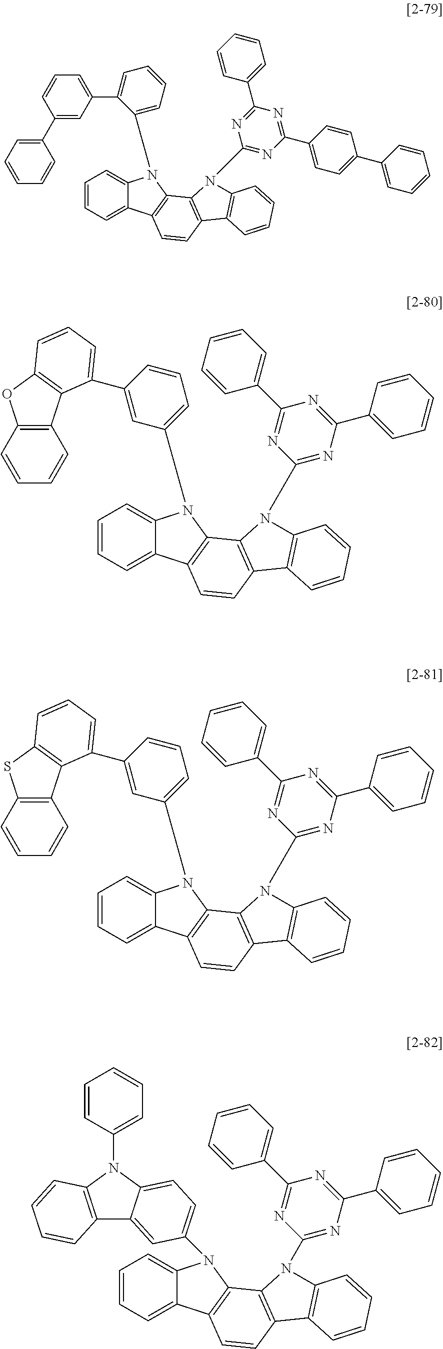 [SMILES: c1ccc(-c2ccc(-c3nc(-c4ccccc4)nc(-n4c5ccccc5c5ccc6c7ccccc7n(-c7ccccc7-c7cccc(-c8ccccc8)c7)c6c54)n3)cc2)cc1 ; c1ccc(-c2nc(-c3ccccc3)nc(-n3c4ccccc4c4ccc5c6ccccc6n(-c6cccc(-c7cccc8oc9ccccc9c78)c6)c5c43)n2)cc1 ; c1ccc(-c2nc(-c3ccccc3)nc(-n3c4ccccc4c4ccc5c6ccccc6n(-c6cccc(-c7cccc8sc9ccccc9c78)c6)c5c43)n2)cc1 ; c1ccc(-c2nc(-c3ccccc3)nc(-n3c4ccccc4c4ccc5c6ccccc6n(-c6ccc7c(c6)c6ccccc6n7-c6ccccc6)c5c43)n2)cc1]

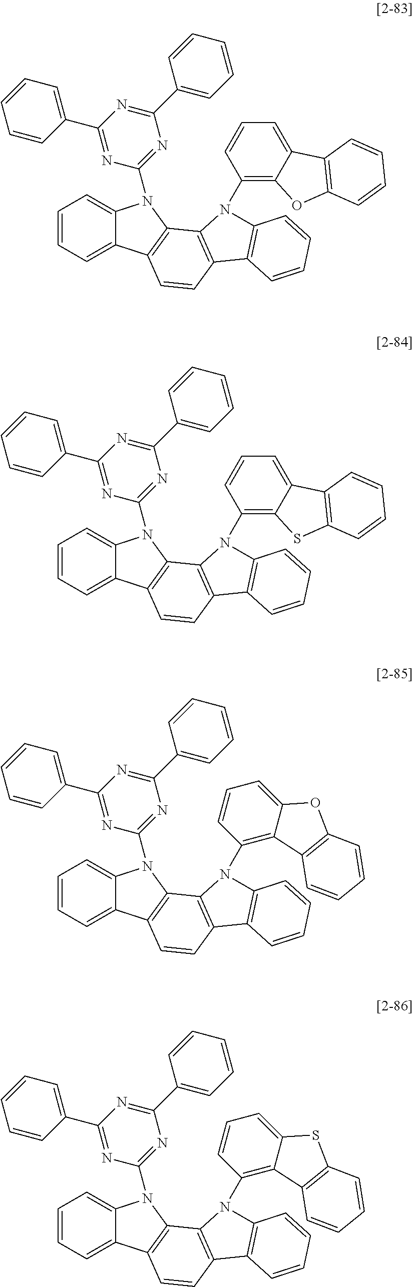 [SMILES: c1ccc(-c2nc(-c3ccccc3)nc(-n3c4ccccc4c4ccc5c6ccccc6n(-c6cccc7c6oc6ccccc67)c5c43)n2)cc1 ; c1ccc(-c2nc(-c3ccccc3)nc(-n3c4ccccc4c4ccc5c6ccccc6n(-c6cccc7c6sc6ccccc67)c5c43)n2)cc1 ; c1ccc(-c2nc(-c3ccccc3)nc(-n3c4ccccc4c4ccc5c6ccccc6n(-c6cccc7oc8ccccc8c67)c5c43)n2)cc1 ; c1ccc(-c2nc(-c3ccccc3)nc(-n3c4ccccc4c4ccc5c6ccccc6n(-c6cccc7sc8ccccc8c67)c5c43)n2)cc1]

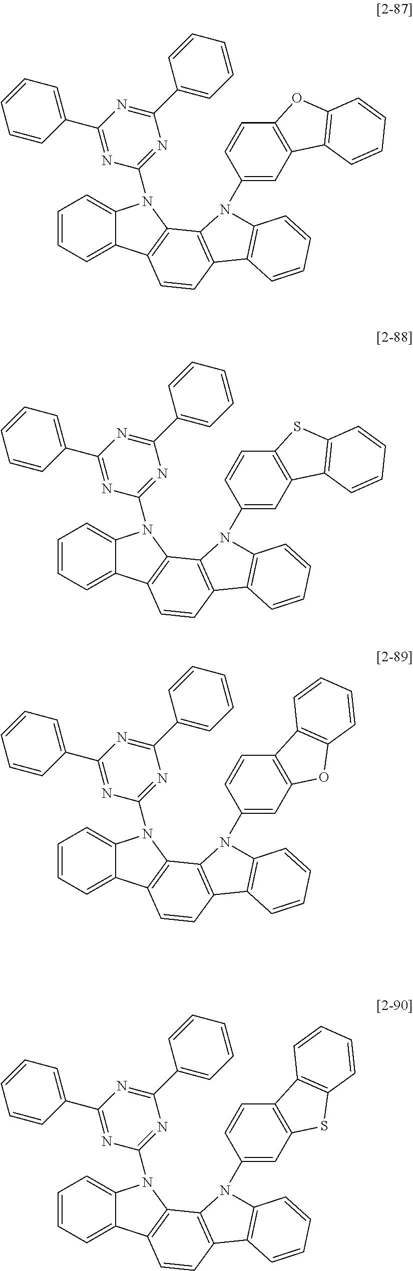 [SMILES: c1ccc(-c2nc(-c3ccccc3)nc(-n3c4ccccc4c4ccc5c6ccccc6n(-c6ccc7oc8ccccc8c7c6)c5c43)n2)cc1 ; c1ccc(-c2nc(-c3ccccc3)nc(-n3c4ccccc4c4ccc5c6ccccc6n(-c6ccc7sc8ccccc8c7c6)c5c43)n2)cc1 ; c1ccc(-c2nc(-c3ccccc3)nc(-n3c4ccccc4c4ccc5c6ccccc6n(-c6ccc7c(c6)oc6ccccc67)c5c43)n2)cc1 ; c1ccc(-c2nc(-c3ccccc3)nc(-n3c4ccccc4c4ccc5c6ccccc6n(-c6ccc7c(c6)sc6ccccc67)c5c43)n2)cc1]

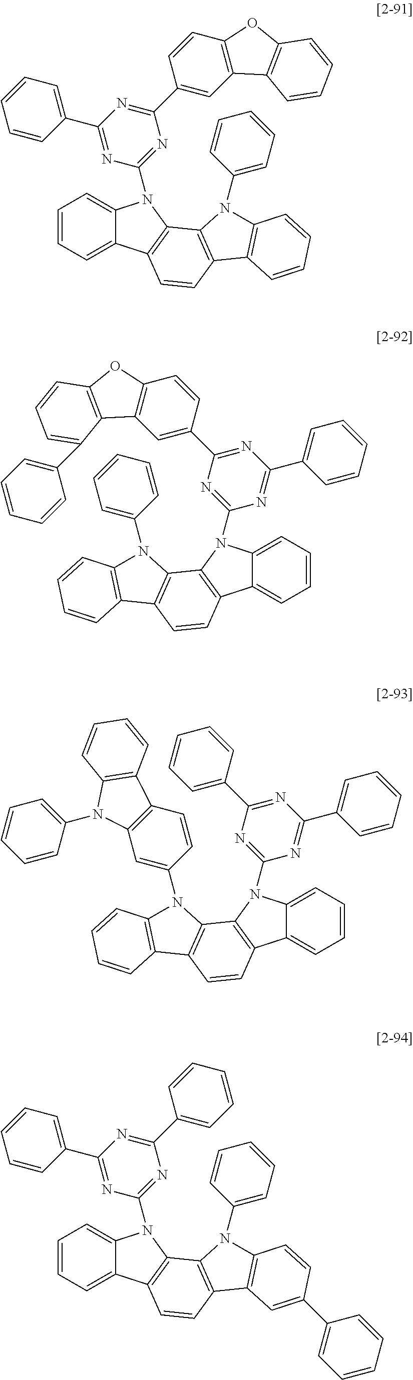 [SMILES: c1ccc(-c2nc(-c3ccc4oc5ccccc5c4c3)nc(-n3c4ccccc4c4ccc5c6ccccc6n(-c6ccccc6)c5c43)n2)cc1 ; c1ccc(-c2nc(-c3ccc4oc5cccc(-c6ccccc6)c5c4c3)nc(-n3c4ccccc4c4ccc5c6ccccc6n(-c6ccccc6)c5c43)n2)cc1 ; c1ccc(-c2nc(-c3ccccc3)nc(-n3c4ccccc4c4ccc5c6ccccc6n(-c6ccc7c8ccccc8n(-c8ccccc8)c7c6)c5c43)n2)cc1 ; c1ccc(-c2ccc3c(c2)c2ccc4c5ccccc5n(-c5nc(-c6ccccc6)nc(-c6ccccc6)n5)c4c2n3-c2ccccc2)cc1]

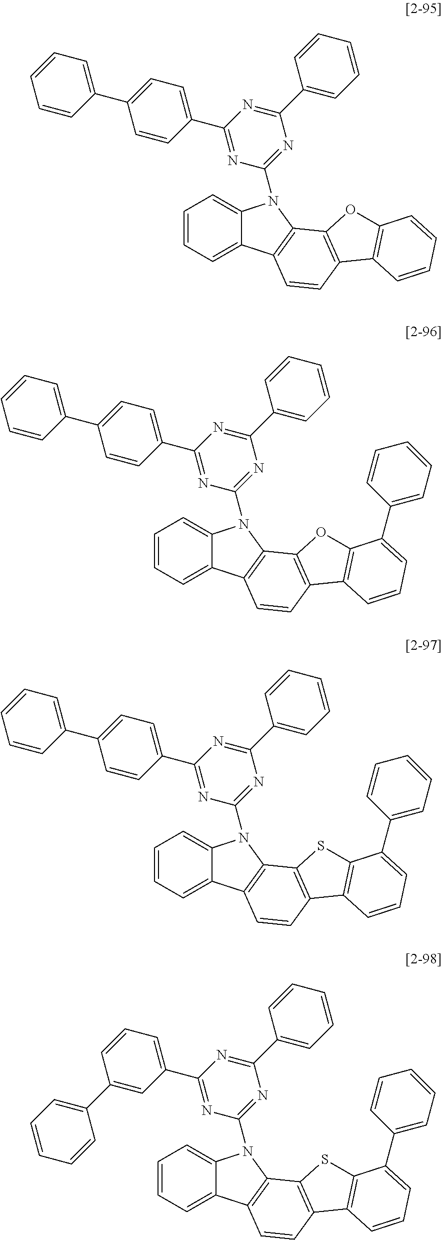 [SMILES: c1ccc(-c2ccc(-c3nc(-c4ccccc4)nc(-n4c5ccccc5c5ccc6c7ccccc7oc6c54)n3)cc2)cc1 ; c1ccc(-c2ccc(-c3nc(-c4ccccc4)nc(-n4c5ccccc5c5ccc6c7cccc(-c8ccccc8)c7oc6c54)n3)cc2)cc1 ; c1ccc(-c2ccc(-c3nc(-c4ccccc4)nc(-n4c5ccccc5c5ccc6c7cccc(-c8ccccc8)c7sc6c54)n3)cc2)cc1 ; c1ccc(-c2cccc(-c3nc(-c4ccccc4)nc(-n4c5ccccc5c5ccc6c7cccc(-c8ccccc8)c7sc6c54)n3)c2)cc1]

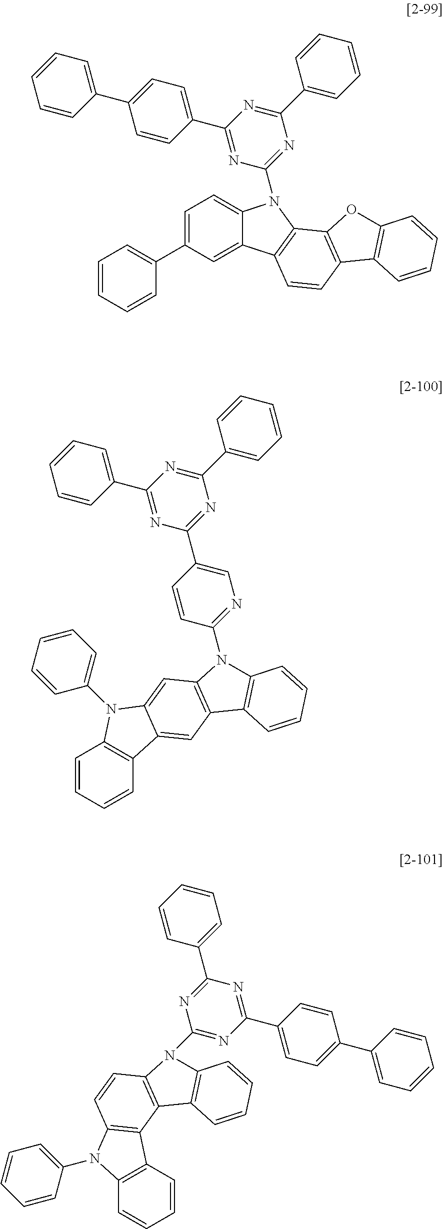 [SMILES: c1ccc(-c2ccc(-c3nc(-c4ccccc4)nc(-n4c5ccc(-c6ccccc6)cc5c5ccc6c7ccccc7oc6c54)n3)cc2)cc1 ; c1ccc(-c2nc(-c3ccccc3)nc(-c3ccc(-n4c5ccccc5c5cc6c7ccccc7n(-c7ccccc7)c6cc54)nc3)n2)cc1 ; c1ccc(-c2ccc(-c3nc(-c4ccccc4)nc(-n4c5ccccc5c5c6c7ccccc7n(-c7ccccc7)c6ccc54)n3)cc2)cc1]

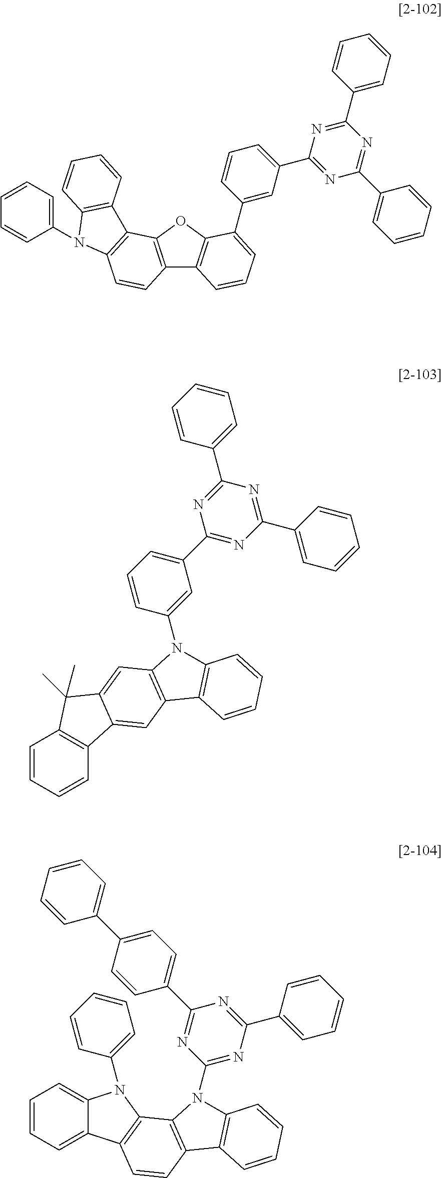 [SMILES: c1ccc(-c2nc(-c3ccccc3)nc(-c3cccc(-c4cccc5c4oc4c5ccc5c4c4ccccc4n5-c4ccccc4)c3)n2)cc1 ; CC1(C)c2ccccc2-c2cc3c4ccccc4n(-c4cccc(-c5nc(-c6ccccc6)nc(-c6ccccc6)n5)c4)c3cc21 ; c1ccc(-c2ccc(-c3nc(-c4ccccc4)nc(-n4c5ccccc5c5ccc6c7ccccc7n(-c7ccccc7)c6c54)n3)cc2)cc1]

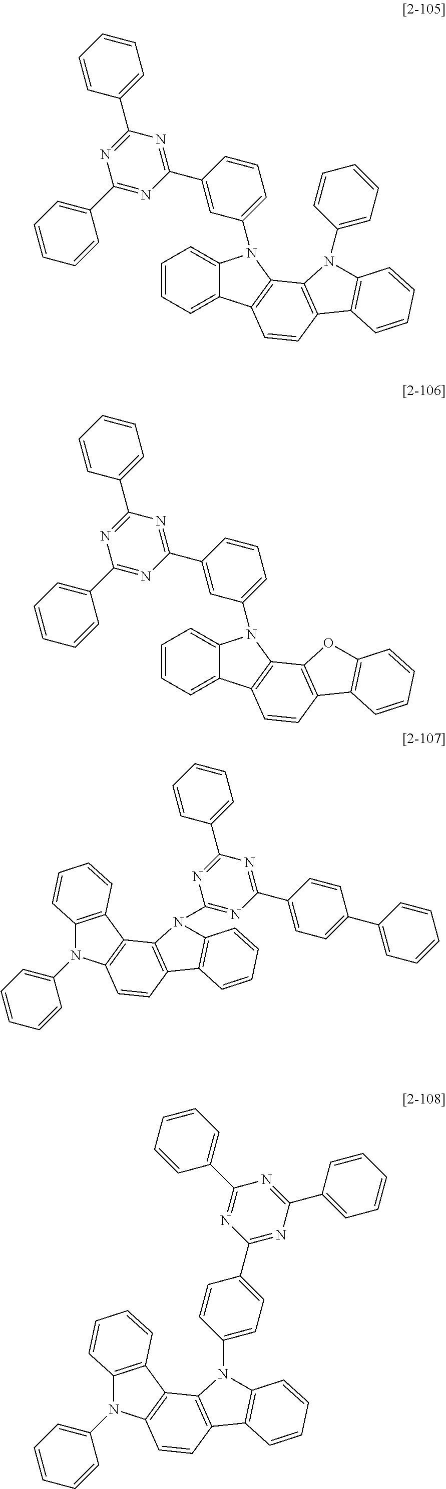 [SMILES: c1ccc(-c2nc(-c3ccccc3)nc(-c3cccc(-n4c5ccccc5c5ccc6c7ccccc7n(-c7ccccc7)c6c54)c3)n2)cc1 ; c1ccc(-c2nc(-c3ccccc3)nc(-c3cccc(-n4c5ccccc5c5ccc6c7ccccc7oc6c54)c3)n2)cc1 ; c1ccc(-c2ccc(-c3nc(-c4ccccc4)nc(-n4c5ccccc5c5ccc6c(c7ccccc7n6-c6ccccc6)c54)n3)cc2)cc1 ; c1ccc(-c2nc(-c3ccccc3)nc(-c3ccc(-n4c5ccccc5c5ccc6c(c7ccccc7n6-c6ccccc6)c54)cc3)n2)cc1]

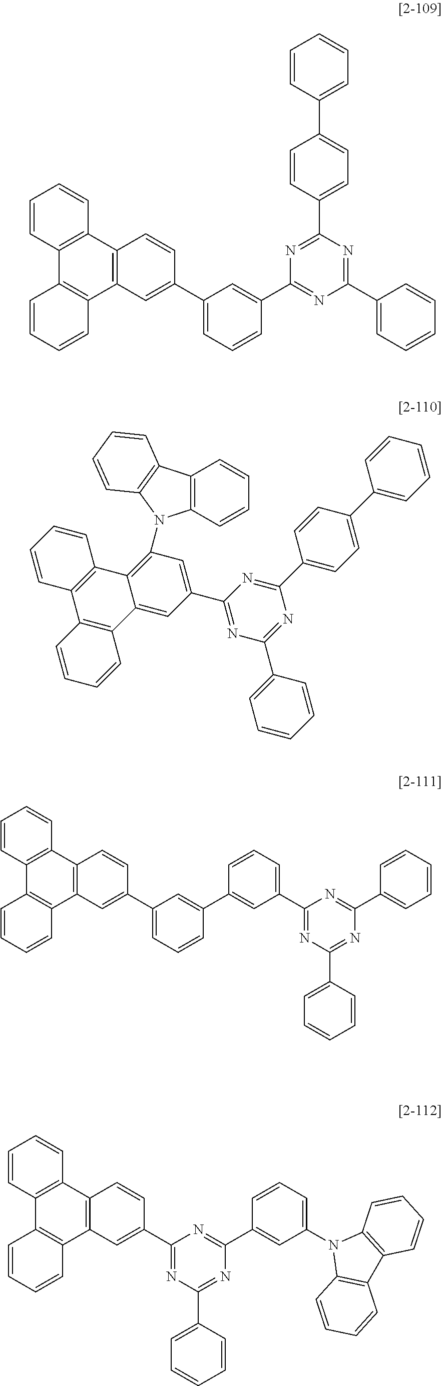 [SMILES: c1ccc(-c2ccc(-c3nc(-c4ccccc4)nc(-c4cccc(-c5ccc6c7ccccc7c7ccccc7c6c5)c4)n3)cc2)cc1 ; c1ccc(-c2ccc(-c3nc(-c4ccccc4)nc(-c4cc(-n5c6ccccc6c6ccccc65)c5c6ccccc6c6ccccc6c5c4)n3)cc2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2nc(-c3cccc(-n4c5ccccc5c5ccccc54)c3)nc(-c3ccc4c5ccccc5c5ccccc5c4c3)n2)cc1]

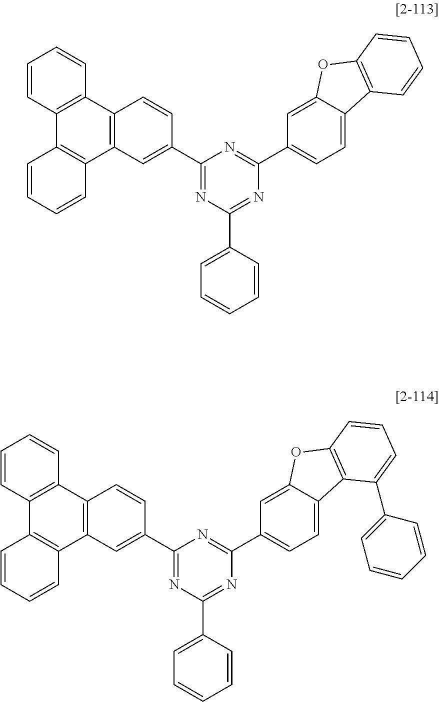 [SMILES: c1ccc(-c2nc(-c3ccc4c(c3)oc3ccccc34)nc(-c3ccc4c5ccccc5c5ccccc5c4c3)n2)cc1 ; c1ccc(-c2nc(-c3ccc4c(c3)oc3cccc(-c5ccccc5)c34)nc(-c3ccc4c5ccccc5c5ccccc5c4c3)n2)cc1]

The first compound and the second compound may be included or mixed, e.g., in a weight ratio of about 1:99 to about 99:1. Within the above range, an appropriate weight ratio may be adjusted using the electron transport capability of the first compound and the hole transport capability of the second compound to implement bipolar characteristics and to improve the efficiency and life-span. Within the above range, e.g., they may be included in a weight ratio of about 10:90 to about 90:10, about 20:80 to about 80:20, for example about 20:80 to about 70:30, about 20:80 to about 60:40, and about 30:70 to about 60:40. In an implementation, they may be included in a weight ratio of about 40:60, about 50:50, or about 60:40.

In addition to the aforementioned compound for an organic optoelectronic device, one or more compounds may be further included.

In an implementation, the composition may further include a dopant.

The dopant may be, e.g., a phosphorescent dopant, such as a red, green, or blue phosphorescent dopant. In an implementation, the dopant may be, e.g., a red or green phosphorescent dopant.

A dopant is a material that emits light by being mixed in a small amount with a compound or composition for an organic optoelectronic device. In general, the dopant may be a material such as a metal complex that emits light by multiple excitation into a triplet or more. The dopant may be, e.g., an inorganic, organic, or organic-inorganic compound, and may include one or two or more.

An example of the dopant may be a phosphorescent dopant, and examples of the phosphorescent dopant may include an organometallic compound including Ir, Pt, Os, Ti, Zr, Hf, Eu, Tb, Tm, Fe, Co, Ni, Ru, Rh, Pd, or a combination thereof. The phosphorescent dopant may include, e.g., a compound represented by Chemical Formula Z.

L¹⁵MX⁵ [Chemical Formula Z]

In Chemical Formula Z, M may be, e.g., a metal, and L¹⁵ and X⁵ may each independently be, e.g., ligands forming a complex with M.

M may be, e.g., Ir, Pt, Os, Ti, Zr, Hf, Eu, Tb, Tm, Fe, Co, Ni, Ru, Rh, Pd, or a combination thereof, and L¹⁵ and X⁵ may be, e.g., a bidentate ligand.

The ligands represented by L¹⁵ and X⁵ may include, e.g., a ligand of Group A.

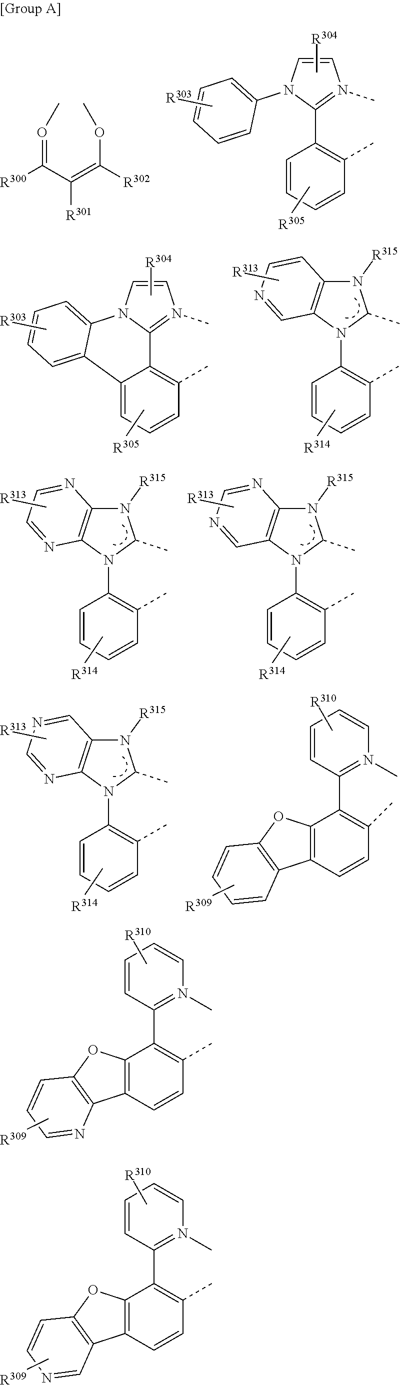

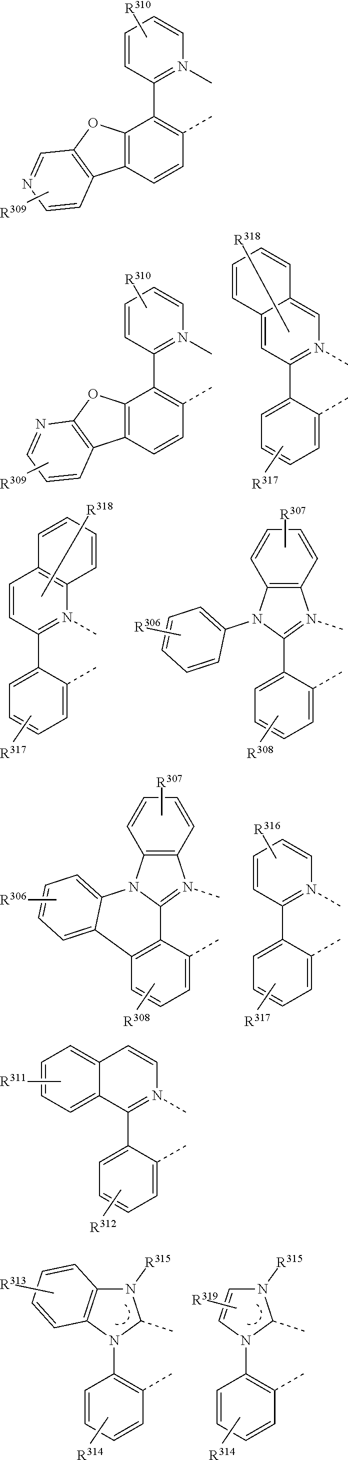

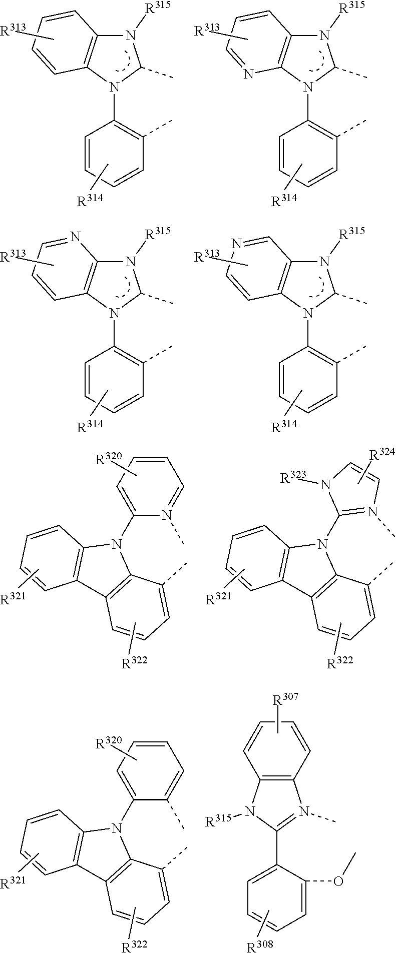

In Group A, R³⁰⁰ to R³⁰² may each independently be, e.g., hydrogen, deuterium a C1 to C30 alkyl group that is substituted or unsubstituted with a halogen, a C6 to C30 aryl group that is substituted or unsubstituted with a C1 to C30 alkyl, or a halogen.

R³⁰³ to R³²⁴ may each independently be, e.g., hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SFs, a trialkylsilyl group having a substituted or unsubstituted C1 to C30 alkyl group, a dialkylarylsilyl group having a substituted or unsubstituted C1 to C30 alkyl group and C6 to C30 aryl group, or a triarylsilyl group having a substituted or unsubstituted C6 to C30 aryl group.

In an implementation, a dopant represented by Chemical Formula V may be included.

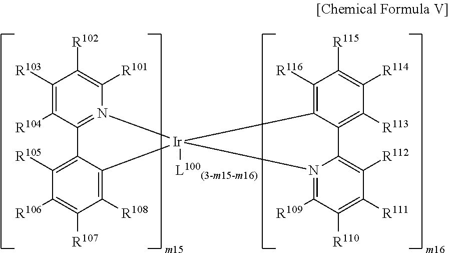

In Chemical Formula V, R¹⁰¹ to R¹¹⁶ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., C1 to C6 alkyl group.

In an implementation, at least one of R¹⁰¹ to R¹¹⁶ may be, e.g., a functional group represented by Chemical Formula V-1.

L¹⁰⁰ may be, e.g., a bidentate ligand of a monovalent anion, and is a ligand that coordinates to iridium through a lone pair of carbons or heteroatoms.

m15 and m16 may each independently be, e.g., an integer of 0 to 3 such that m15+m16 is an integer of 1 to 3.

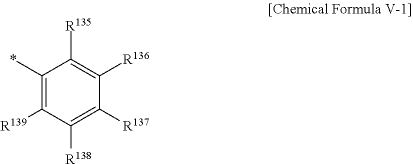

In Chemical Formula V-1, R¹³⁵ to R¹³⁹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., C1 to C6 alkyl group.

* indicates a portion linked to a carbon atom.

In an implementation, a dopant represented by, e.g., Chemical Formula Z-1 may be included.

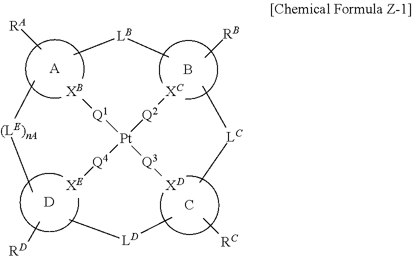

In Chemical Formula Z-1, rings A, B, C, and D may each independently represent a 5-membered or 6-membered carbocyclic or heterocyclic ring.

R^{A}, R^{B}, R^{C}, and R^{D} may each independently represent mono-, di-, tri-, or tetra-substitution, or unsubstitution.

L^{B}, L^{C}, and L^{D} may each independently be, e.g., a direct bond, BR, NR, PR, 0, S, Se, C═O, S═O, SO₂, CRR′, SiRR′, GeRR′, or a combination thereof.

In an implementation, when nA is 1, L^{E} may be, e.g., a direct bond, BR, NR, PR, 0, S, Se, C═O, S═O, SO₂, CRR′, SiRR′, GeRR′, and a combination thereof, when nA is 0, L^{E} does not exist.

R^{A}, R^{B}, R^{C}, R^{D}, R, and R′ may each independently be, e.g., hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}, R^{B}, R^{C}, R^{D}, R, and R′ may be optionally linked to each other to provide a ring; X^{B}, X^{C}, X^{D}, and X^{E} may each independently be, e.g., carbon or nitrogen; and Q¹, Q², Q³, and Q⁴ may each independently be, e.g., oxygen or a direct bond.

The dopant according to an embodiment may be a platinum complex, and may be, e.g., represented by Chemical Formula VI.

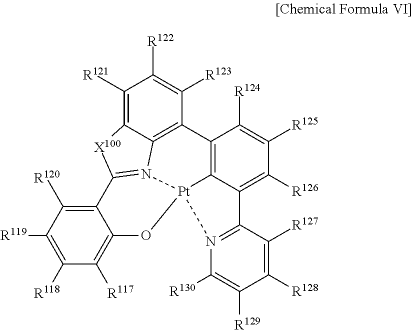

In Chemical Formula VI, X¹⁰⁰ may be, e.g., O, S, or NR¹³¹.

R¹¹⁷ to R¹³¹ may each independently be, e.g., hydrogen, deuterium, a substituted or unsubstituted C1 to C10 alkyl group, a substituted or unsubstituted C6 to C20 aryl group, or —SiR¹³²R¹³³R¹³⁴.

R¹³² to R¹³⁴ may each independently be, e.g., C1 to C6 alkyl group.

In an implementation, at least one of R¹¹⁷ to R¹³¹ may be, e.g., —SiR¹³²R¹³³R¹³⁴ or tert-butyl group.

Hereinafter, an organic optoelectronic device including the aforementioned compound for an organic optoelectronic device or composition for an organic optoelectronic device is described.

The organic optoelectronic device may be a suitable device to convert electrical energy into photoenergy and vice versa without particular limitation, and may be, e.g., an organic photoelectric device, an organic light emitting diode, an organic solar cell, or an organic photoconductor drum.

Herein, an organic light emitting diode as one example of an organic optoelectronic device is described referring to the drawing.

The FIGURE is a cross-sectional view of an organic light emitting diode according to an embodiment.

Referring to the FIGURE, an organic light emitting diode 100 according to an embodiment may include an anode 120 and a cathode 110 facing each other and an organic layer 105 disposed between the anode 120 and cathode 110.

The anode 120 may be made of a conductor having a large work function to help hole injection, and may be, e.g., a metal, a metal oxide, or a conductive polymer. The anode 120 may be, e.g., a metal such as nickel, platinum, vanadium, chromium, copper, zinc, gold, or the like, or an alloy thereof; a metal oxide such as zinc oxide, indium oxide, indium tin oxide (ITO), indium zinc oxide (IZO), or the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, or polyaniline.

The cathode 110 may be made of a conductor having a small work function to help electron injection, and may be, e.g., a metal, a metal oxide, or a conductive polymer. The cathode 110 may be, e.g., a metal such as magnesium, calcium, sodium, potassium, titanium, indium, yttrium, lithium, gadolinium, aluminum silver, tin, lead, cesium, barium, or the like, or an alloy thereof; a multi-layer structure material such as LiF/Al, LiO₂/Al, LiF/Ca, or BaF₂/Ca.

The organic layer 105 may include the aforementioned compound for an organic optoelectronic device or composition for an organic optoelectronic device.

The organic layer 105 may include, e.g., the light emitting layer 130, and the light emitting layer 130 may include, e.g., the aforementioned compound for an organic optoelectronic device or composition for an organic optoelectronic device.

The composition for an organic optoelectronic device further including a dopant may be, e.g., a green light-emitting composition.

The light emitting layer 130 may include, e.g., the aforementioned composition for an organic optoelectronic device as a phosphorescent host.

The organic layer may further include a charge transport region in addition to the light emitting layer.

The charge transport region may be, e.g., the hole transport region 140.

The hole transport region 140 may help further increase hole injection and/or hole mobility between the anode 120 and the light emitting layer 130 and block electrons. In an implementation, the hole transport region 140 may include a hole transport layer between the anode 120 and the light emitting layer 130, and a hole transport auxiliary layer between the light emitting layer 130 and the hole transport layer.

In an implementation, the aforementioned compound for an organic optoelectronic device or composition for an organic optoelectronic device may be included in at least one layer of the hole transport layer and hole transport auxiliary layer.

In an implementation, it may be included in the hole transport auxiliary layer.

In an implementation, a compound of Group B may be included in at least one of the hole transport layer and the hole transport auxiliary layer.

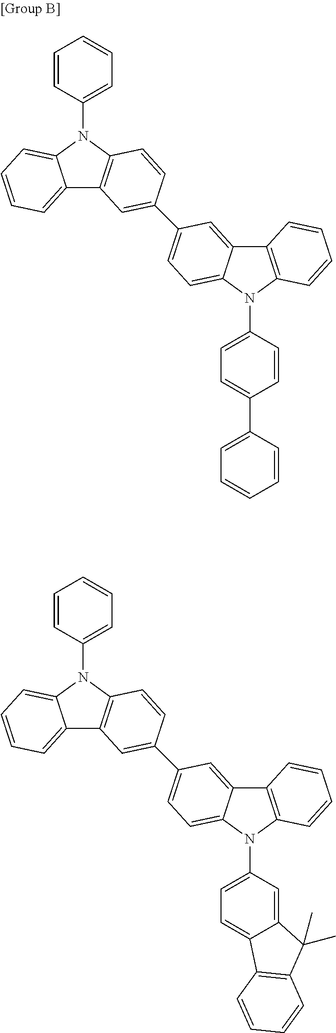 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21]

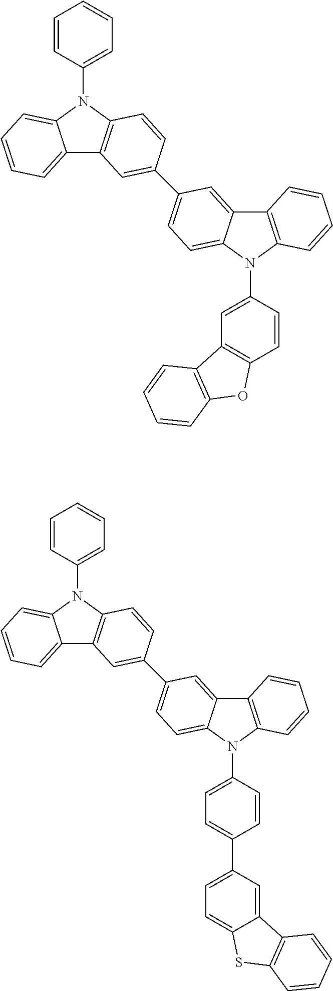 [SMILES: c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1]

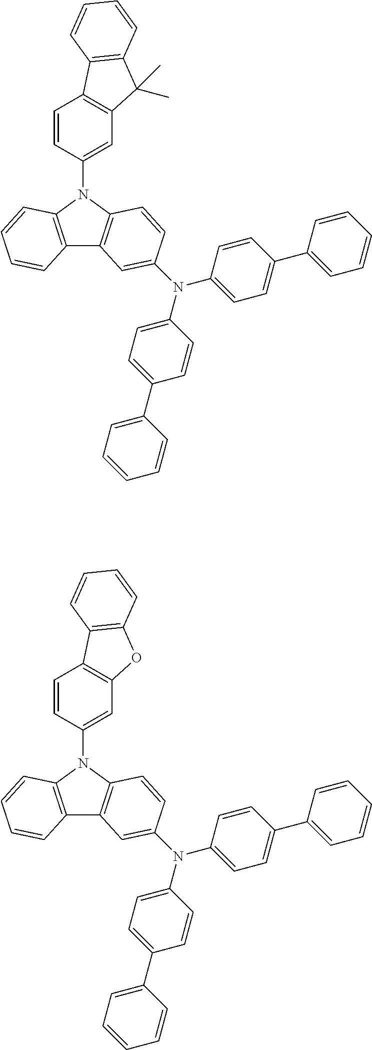 [SMILES: CC1(C)c2ccccc2-c2ccc(-n3c4ccccc4c4cc(N(c5ccc(-c6ccccc6)cc5)c5ccc(-c6ccccc6)cc5)ccc43)cc21 ; c1ccc(-c2ccc(N(c3ccc(-c4ccccc4)cc3)c3ccc4c(c3)c3ccccc3n4-c3ccc4c(c3)oc3ccccc34)cc2)cc1]

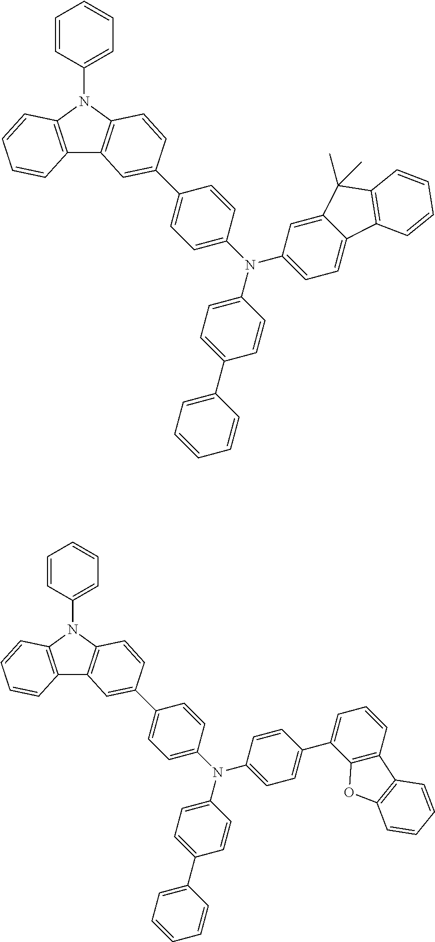 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

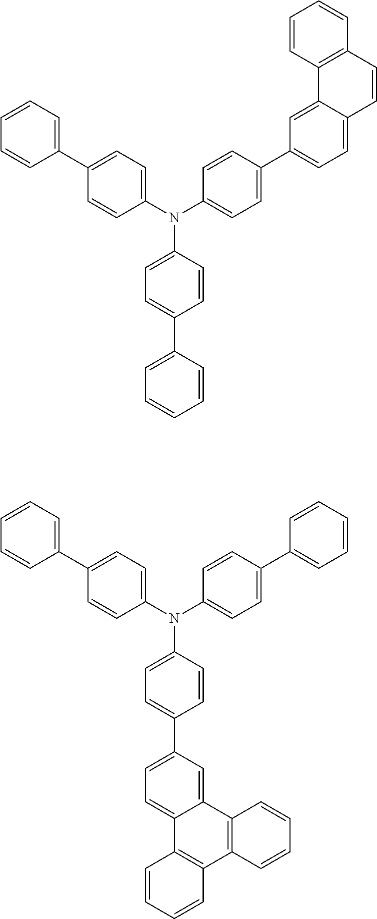 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1]

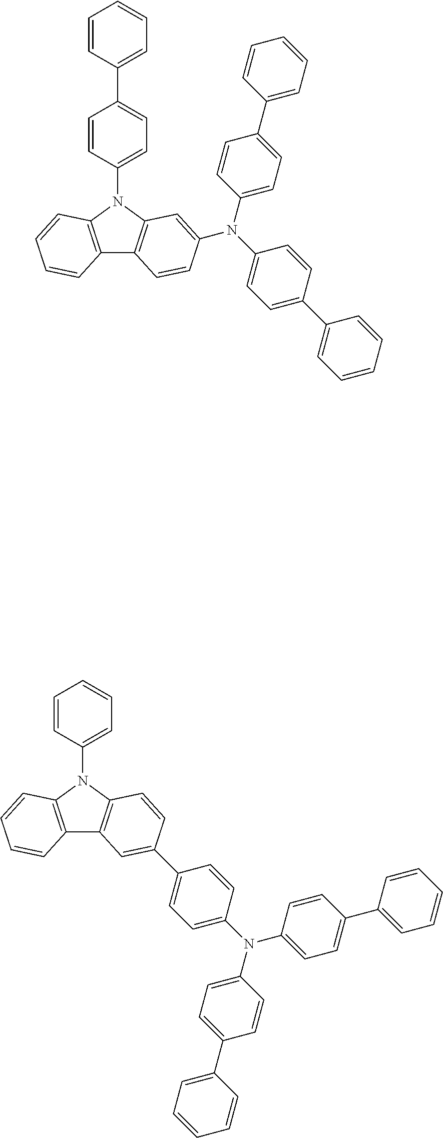 [SMILES: c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

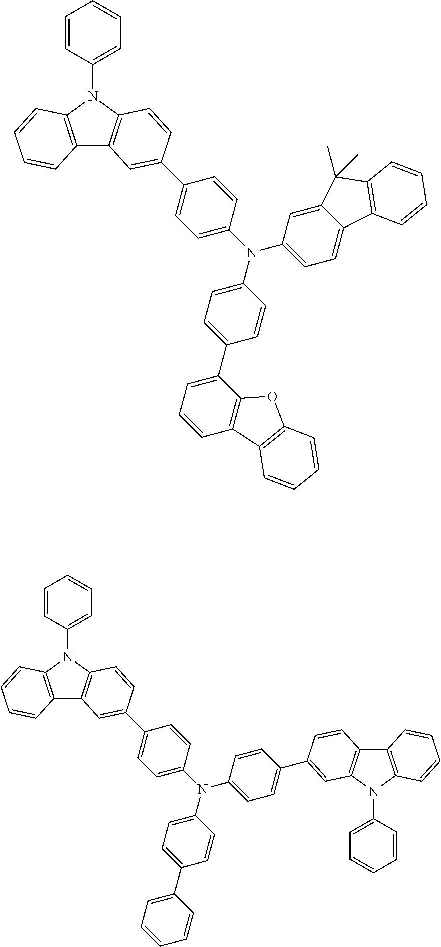 [SMILES: CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

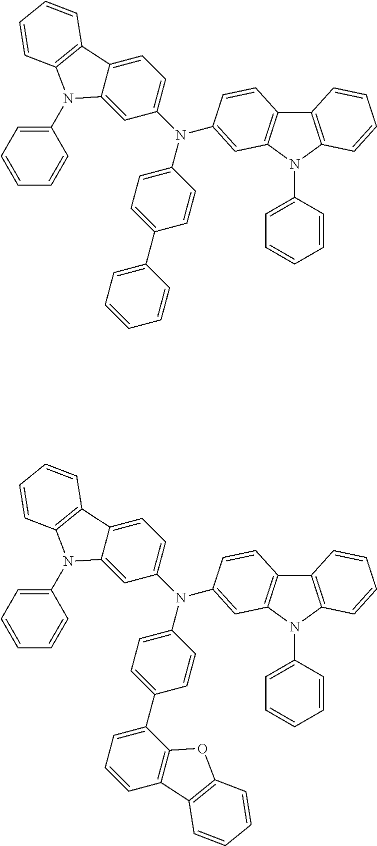 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1]

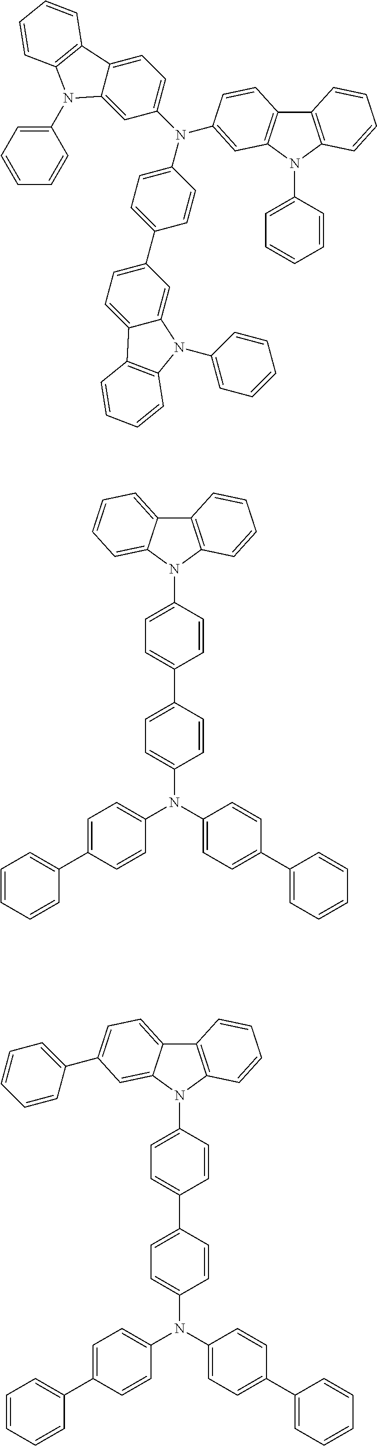 [SMILES: c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1]

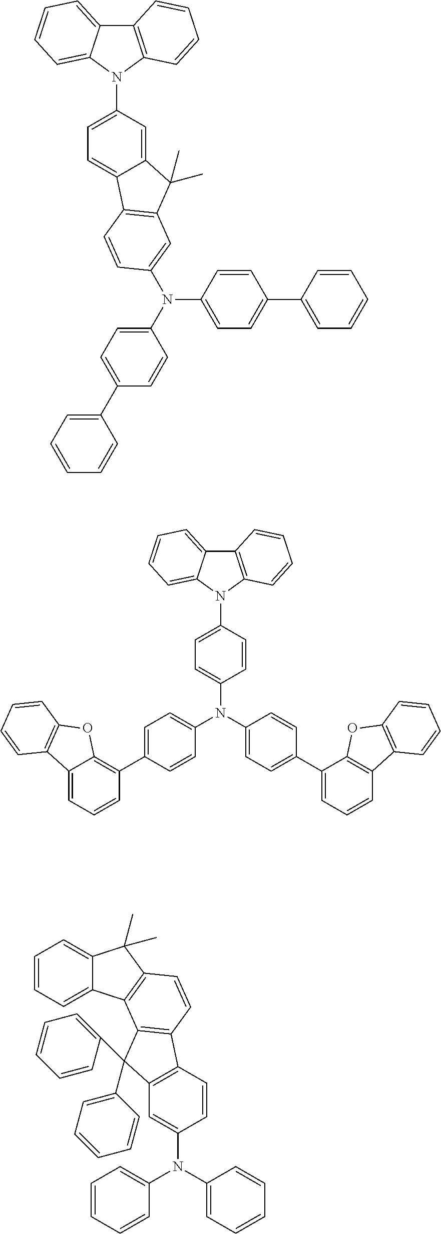 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc2c(c1)oc1c(-c3ccc(N(c4ccc(-c5cccc6c5oc5ccccc56)cc4)c4ccc(-n5c6ccccc6c6ccccc65)cc4)cc3)cccc12 ; CC1(C)c2ccccc2-c2c1ccc1c2C(c2ccccc2)(c2ccccc2)c2cc(N(c3ccccc3)c3ccccc3)ccc2-1]

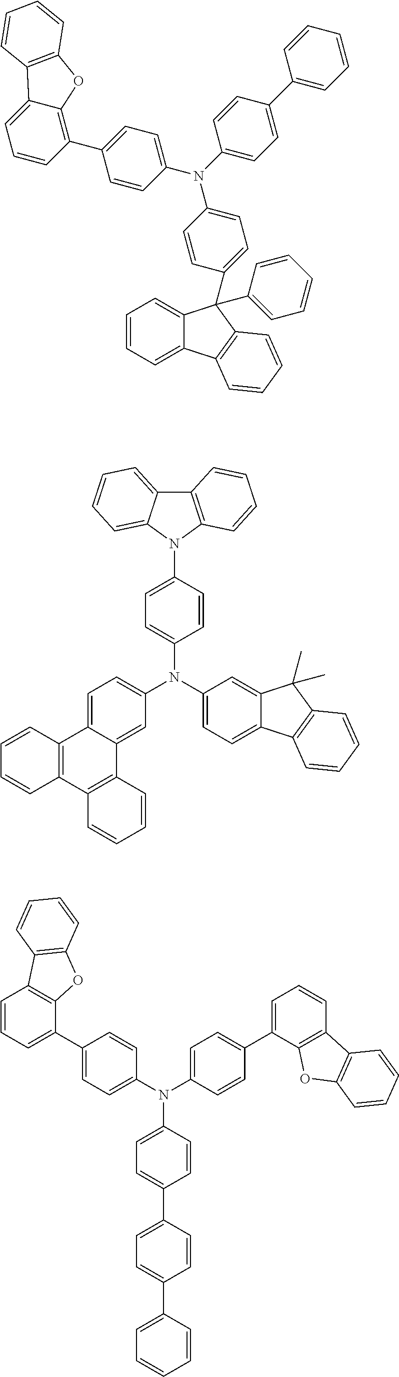 [SMILES: c1ccc(-c2ccc(N(c3ccc(-c4cccc5c4oc4ccccc45)cc3)c3ccc(C4(c5ccccc5)c5ccccc5-c5ccccc54)cc3)cc2)cc1 ; CC1(C)c2ccccc2-c2ccc(N(c3ccc(-n4c5ccccc5c5ccccc54)cc3)c3ccc4c5ccccc5c5ccccc5c4c3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

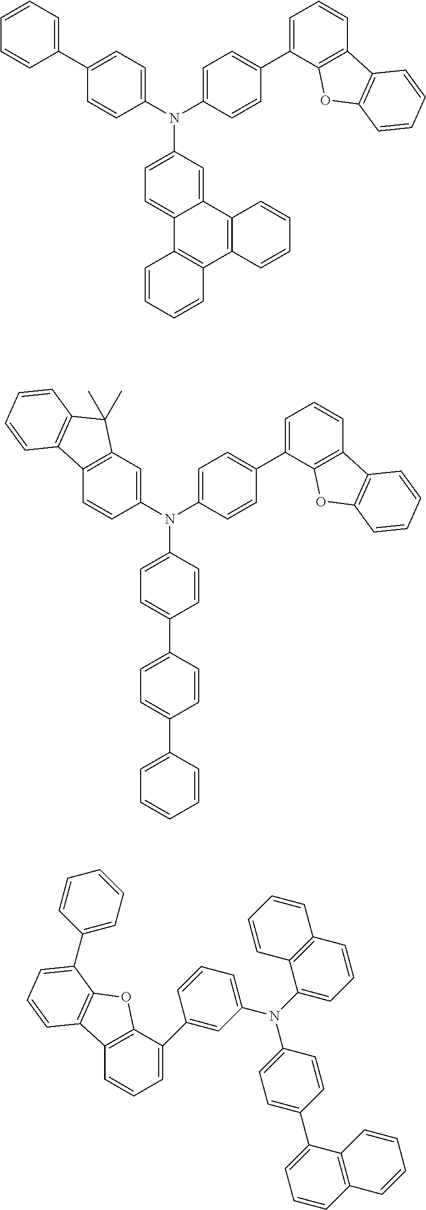 [SMILES: c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21 ; c1ccc(-c2cccc3c2oc2c(-c4cccc(N(c5ccc(-c6cccc7ccccc67)cc5)c5cccc6ccccc56)c4)cccc23)cc1]

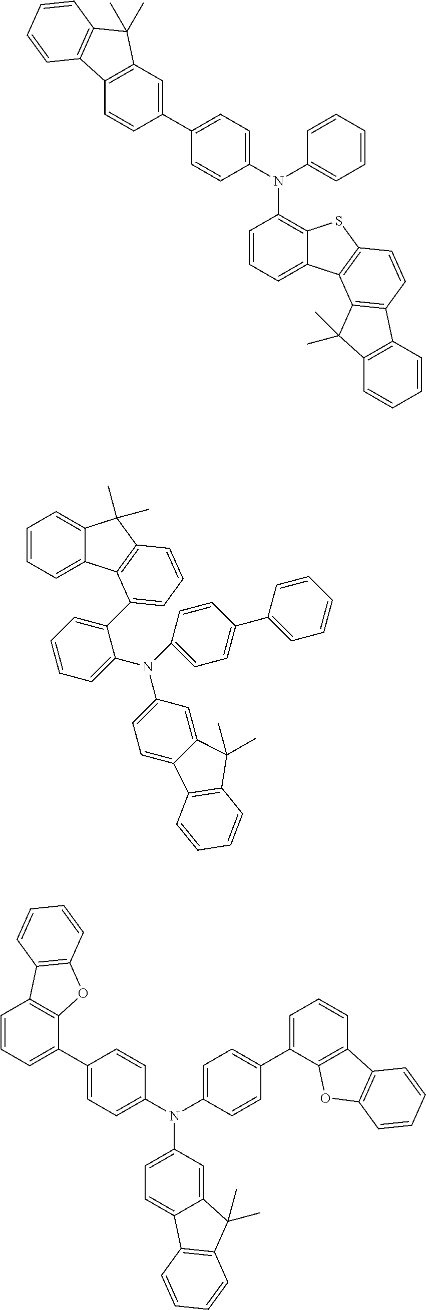 [SMILES: CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21 ; CC1(C)c2ccccc2-c2ccc(N(c3ccc(-c4cccc5c4oc4ccccc45)cc3)c3ccc(-c4cccc5c4oc4ccccc45)cc3)cc21]

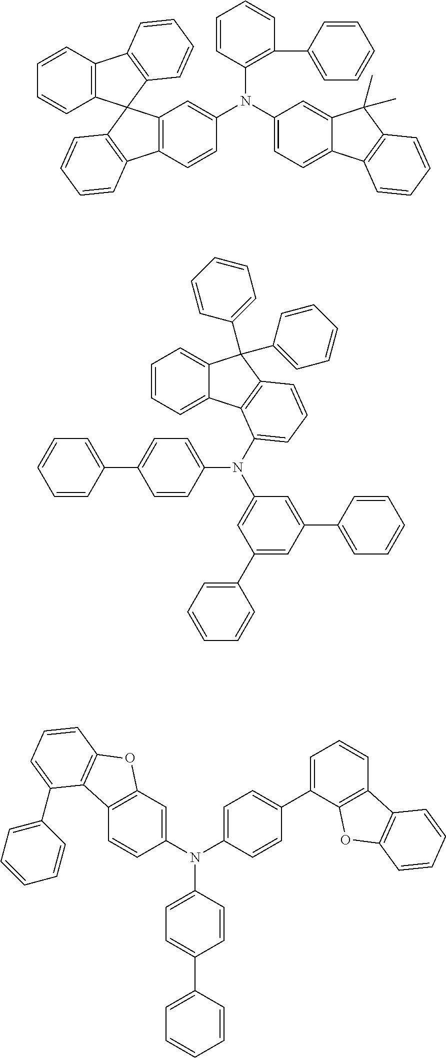 [SMILES: CC1(C)c2ccccc2-c2ccc(N(c3ccc4c(c3)C3(c5ccccc5-c5ccccc53)c3ccccc3-4)c3ccccc3-c3ccccc3)cc21 ; c1ccc(-c2ccc(N(c3cc(-c4ccccc4)cc(-c4ccccc4)c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3ccc4c(c3)oc3cccc(-c5ccccc5)c34)cc2)cc1]

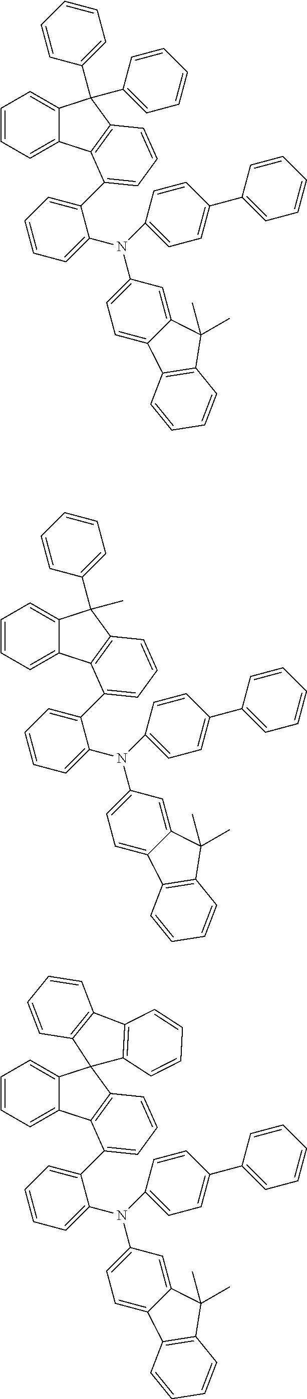 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

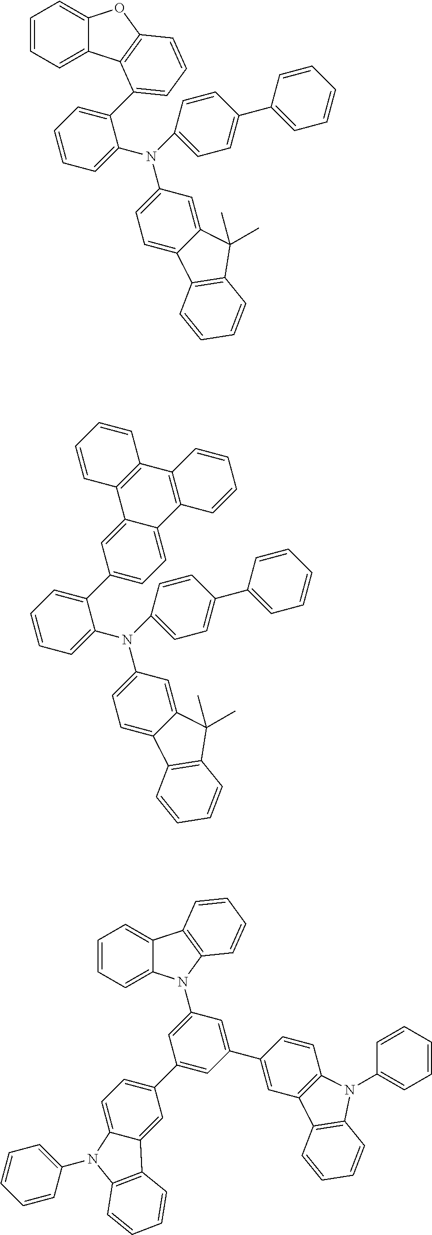 [SMILES: CC1(C)c2ccccc2-c2ccc(N(c3ccc(-c4ccccc4)cc3)c3ccccc3-c3cccc4oc5ccccc5c34)cc21 ; CC1(C)c2ccccc2-c2ccc(N(c3ccc(-c4ccccc4)cc3)c3ccccc3-c3ccc4c5ccccc5c5ccccc5c4c3)cc21 ; c1ccc(-n2c3ccccc3c3cc(-c4cc(-c5ccc6c(c5)c5ccccc5n6-c5ccccc5)cc(-n5c6ccccc6c6ccccc65)c4)ccc32)cc1]

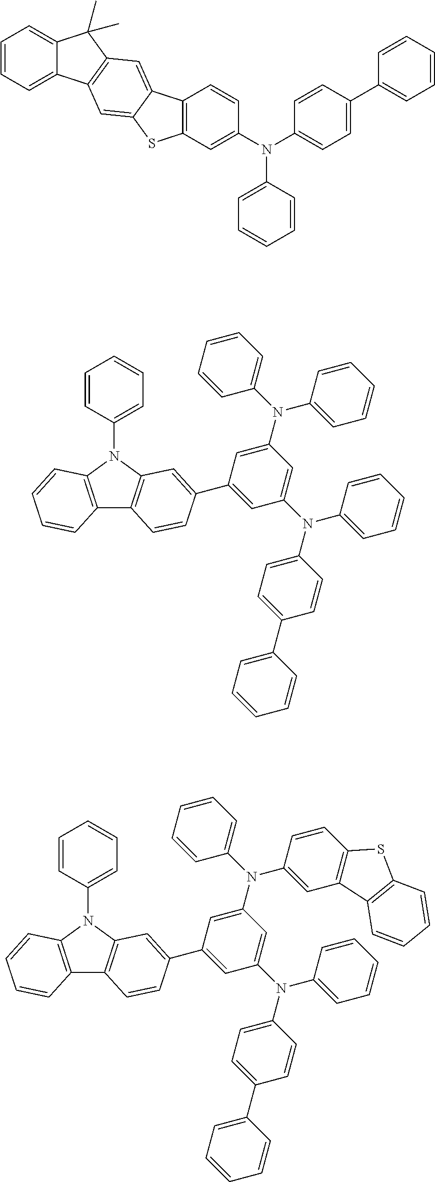 [SMILES: CC1(C)c2ccccc2-c2cc3sc4cc(N(c5ccccc5)c5ccc(-c6ccccc6)cc5)ccc4c3cc21 ; c1ccc(-c2ccc(N(c3ccccc3)c3cc(-c4ccc5c6ccccc6n(-c6ccccc6)c5c4)cc(N(c4ccccc4)c4ccccc4)c3)cc2)cc1 ; c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1]

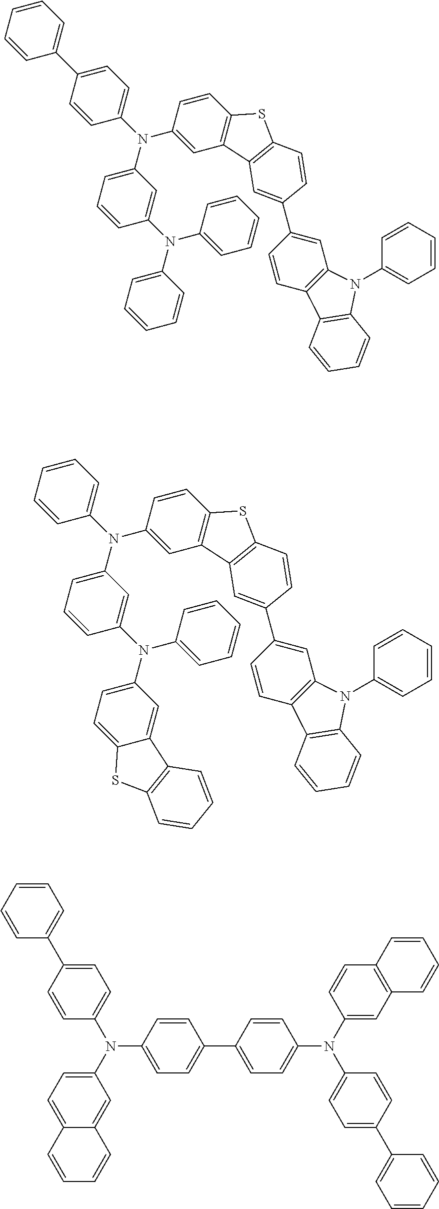 [SMILES: c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1 ; c1ccc(-c2ccc(N(c3ccc(-c4ccc(N(c5ccc(-c6ccccc6)cc5)c5ccc6ccccc6c5)cc4)cc3)c3ccc4ccccc4c3)cc2)cc1]

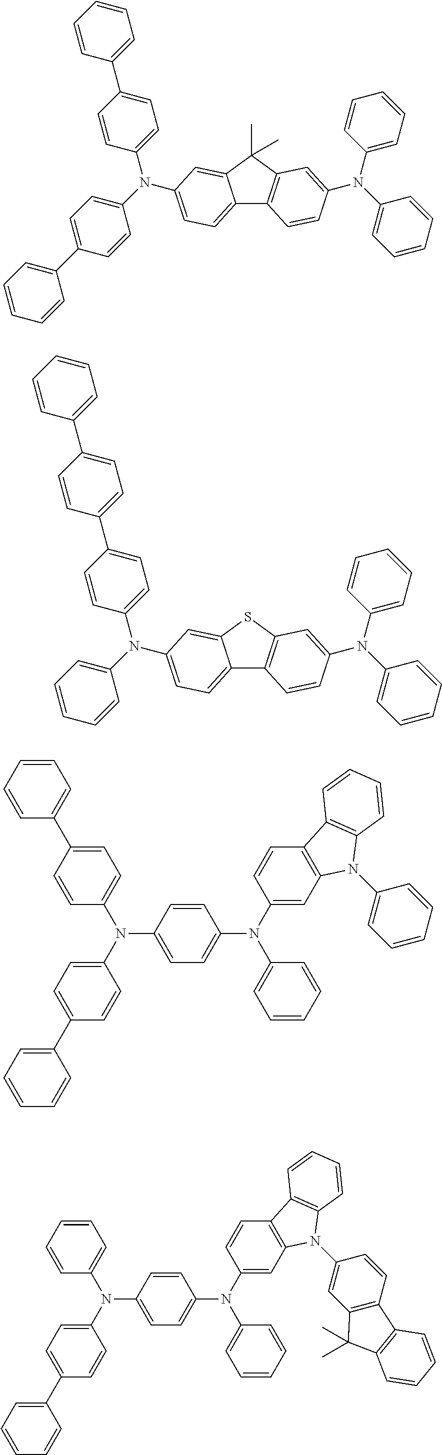

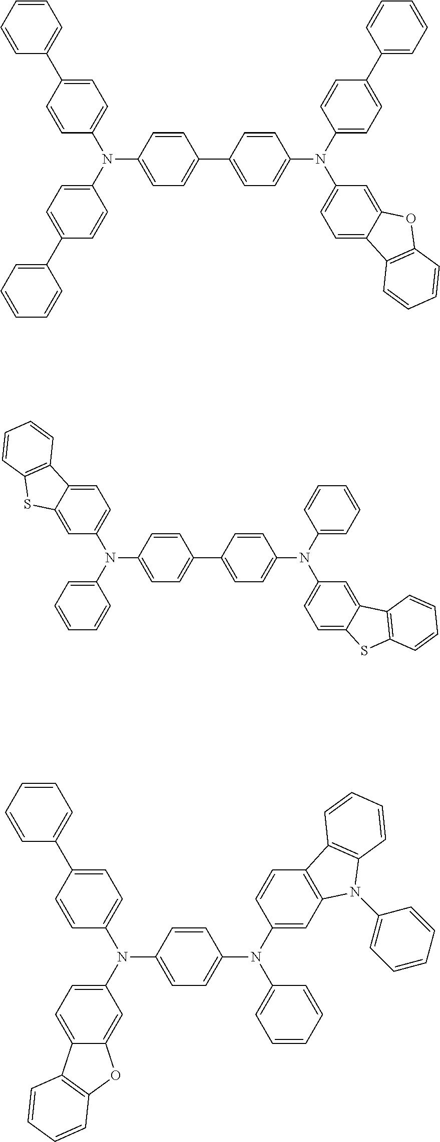 [SMILES: c1ccc(-c2ccc(N(c3ccc(-c4ccccc4)cc3)c3ccc(-c4ccc(N(c5ccc(-c6ccccc6)cc5)c5ccc6c(c5)oc5ccccc56)cc4)cc3)cc2)cc1 ; c1ccc(N(c2ccc(-c3ccc(N(c4ccccc4)c4ccc5sc6ccccc6c5c4)cc3)cc2)c2ccc3c(c2)sc2ccccc23)cc1 ; c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1]

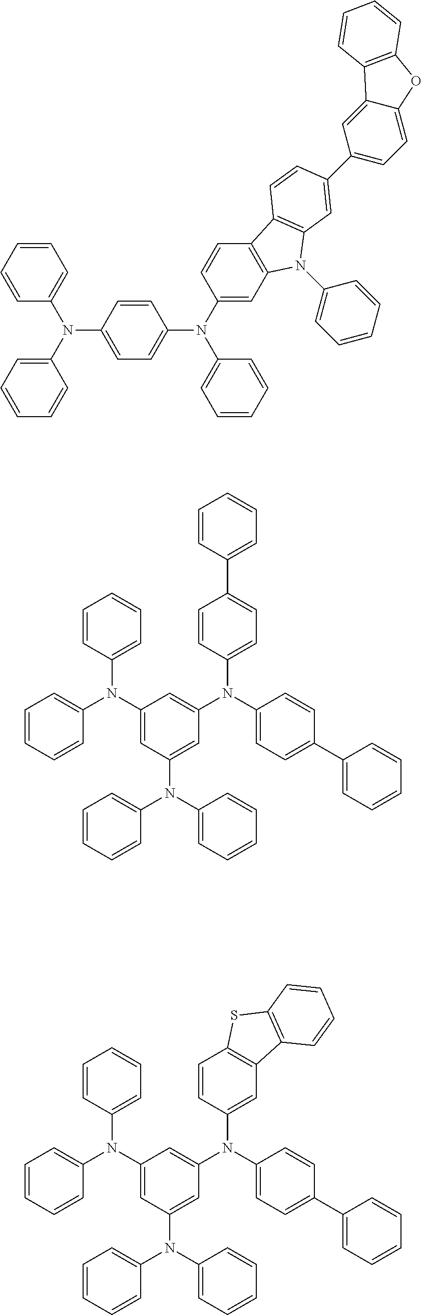 [SMILES: c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1 ; c1ccc(-c2ccc(N(c3cc(N(c4ccccc4)c4ccccc4)cc(N(c4ccccc4)c4ccccc4)c3)c3ccc4sc5ccccc5c4c3)cc2)cc1]

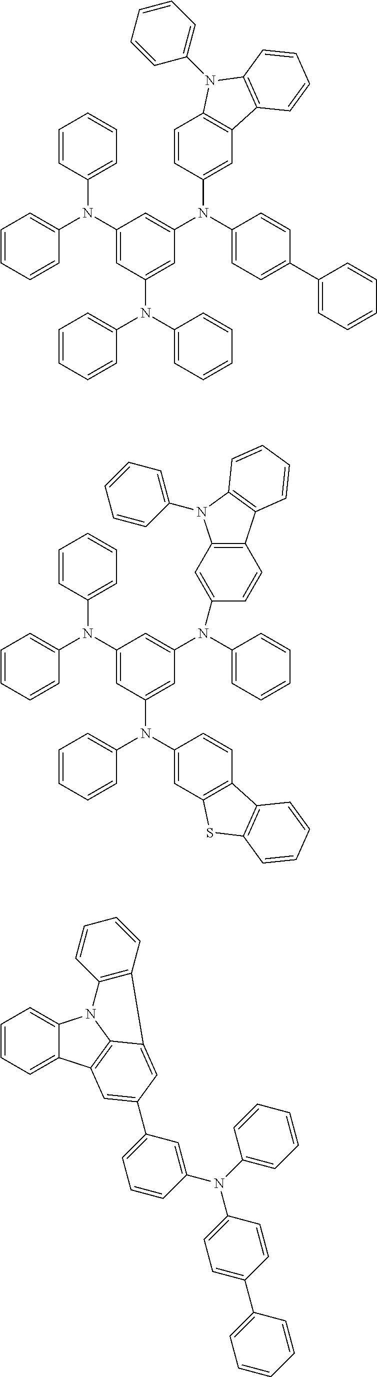 [SMILES: c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1]

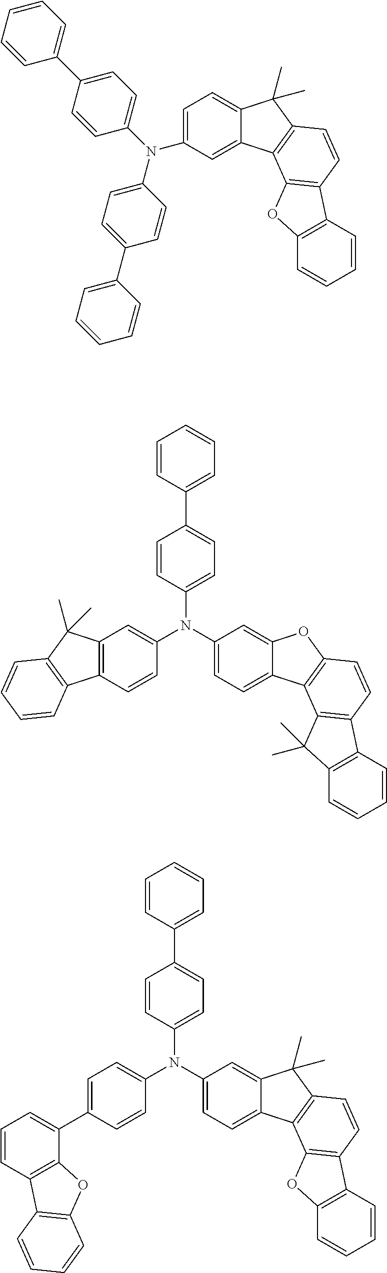 [SMILES: CC1(C)c2ccc(N(c3ccc(-c4ccccc4)cc3)c3ccc(-c4ccccc4)cc3)cc2-c2c1ccc1c2oc2ccccc21 ; CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21]

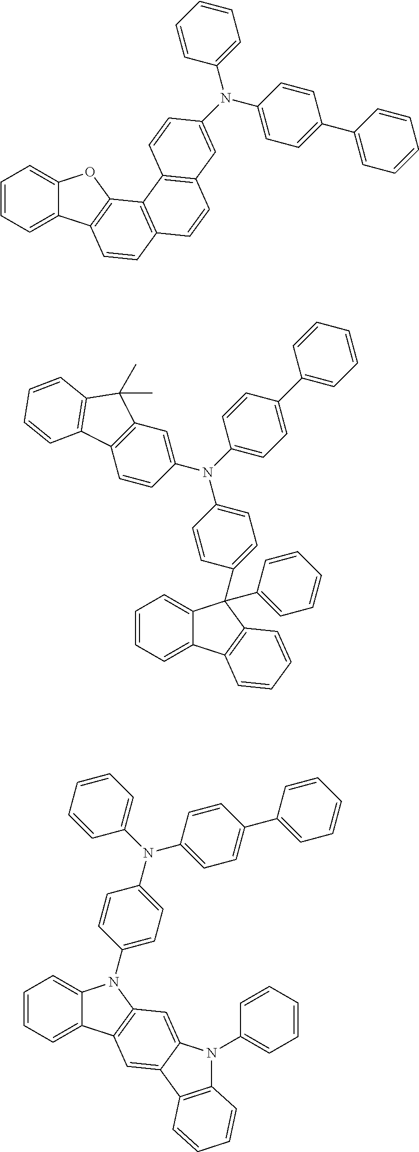 [SMILES: c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21 ; c1ccc(-c2ccc(N(c3ccccc3)c3ccc(-n4c5ccccc5c5cc6c7ccccc7n(-c7ccccc7)c6cc54)cc3)cc2)cc1]

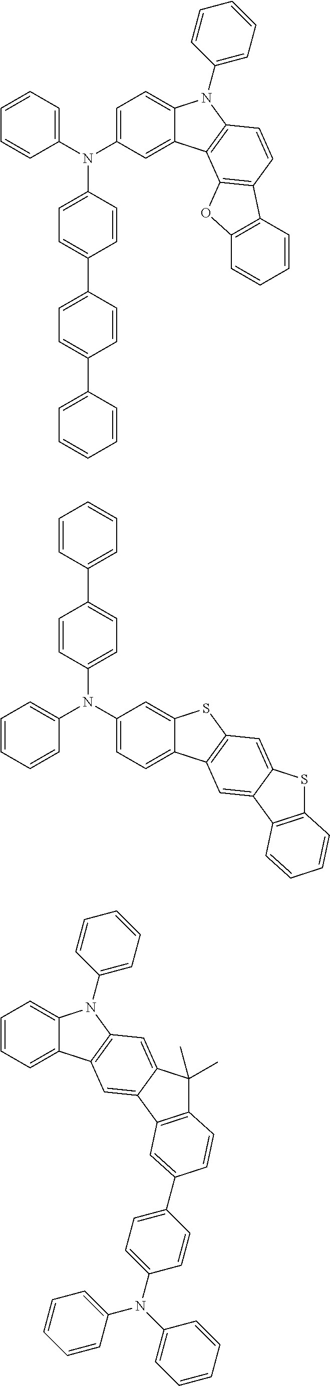

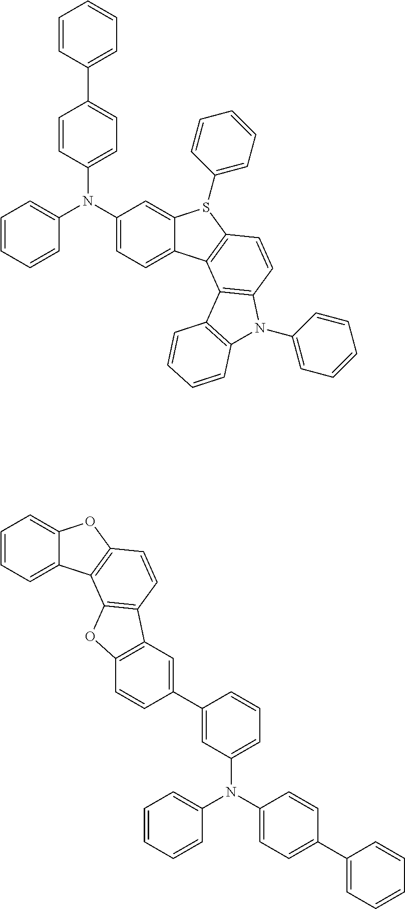

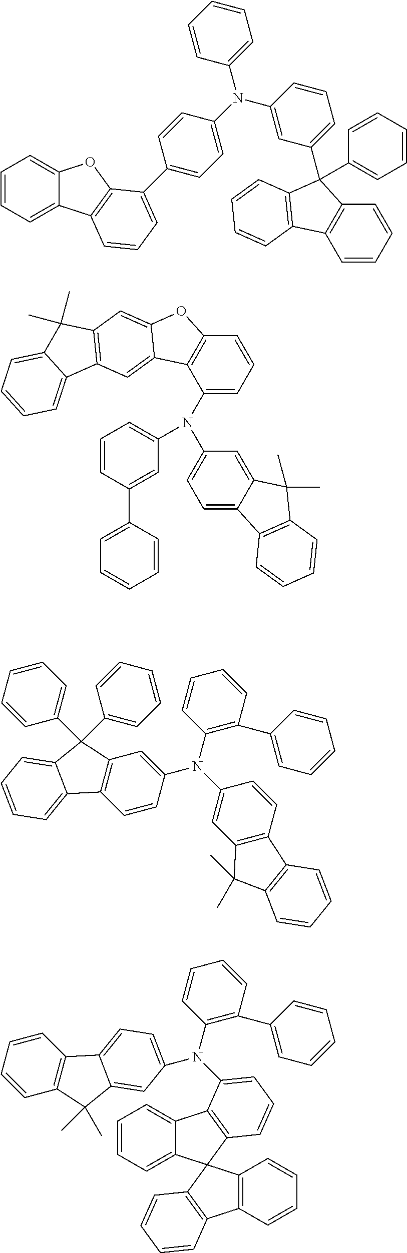 [SMILES: c1ccc(N(c2ccc(-c3cccc4c3oc3ccccc34)cc2)c2cccc(C3(c4ccccc4)c4ccccc4-c4ccccc43)c2)cc1 ; CC1(C)c2ccccc2-c2ccc(N(c3cccc(-c4ccccc4)c3)c3cccc4oc5cc6c(cc5c34)-c3ccccc3C6(C)C)cc21 ; CC1(C)c2ccccc2-c2ccc(N(c3ccc4c(c3)C(c3ccccc3)(c3ccccc3)c3ccccc3-4)c3ccccc3-c3ccccc3)cc21 ; CC1(C)c2ccccc2-c2ccc(N(c3ccccc3-c3ccccc3)c3cccc4c3-c3ccccc3C43c4ccccc4-c4ccccc43)cc21]

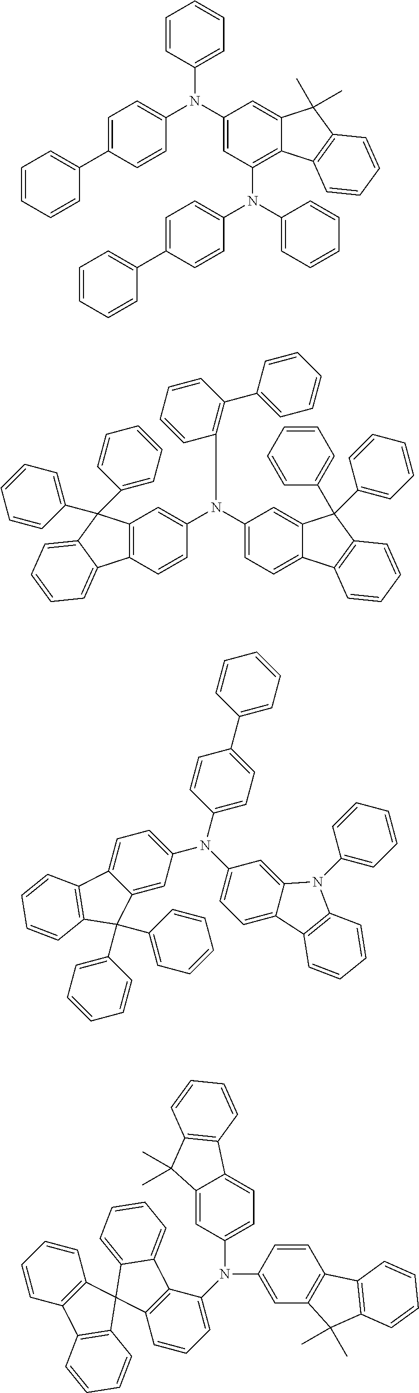 [SMILES: CC1(C)c2ccccc2-c2c(N(c3ccccc3)c3ccc(-c4ccccc4)cc3)cc(N(c3ccccc3)c3ccc(-c4ccccc4)cc3)cc21 ; c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21]

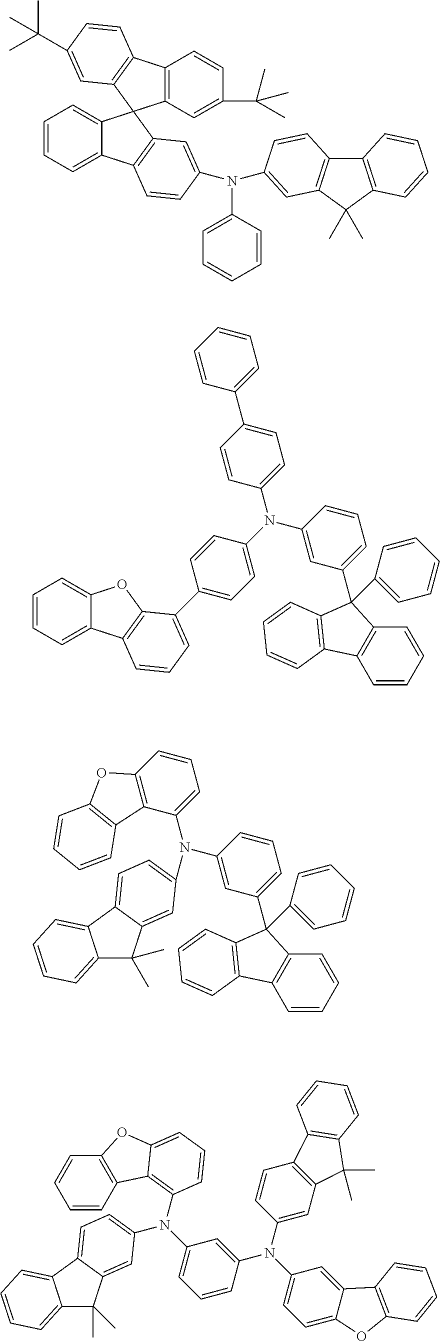 [SMILES: CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2 ; c1ccc(-c2ccc(N(c3ccc(-c4cccc5c4oc4ccccc45)cc3)c3cccc(C4(c5ccccc5)c5ccccc5-c5ccccc54)c3)cc2)cc1 ; CC1(C)c2ccccc2-c2ccc(N(c3cccc(C4(c5ccccc5)c5ccccc5-c5ccccc54)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21]

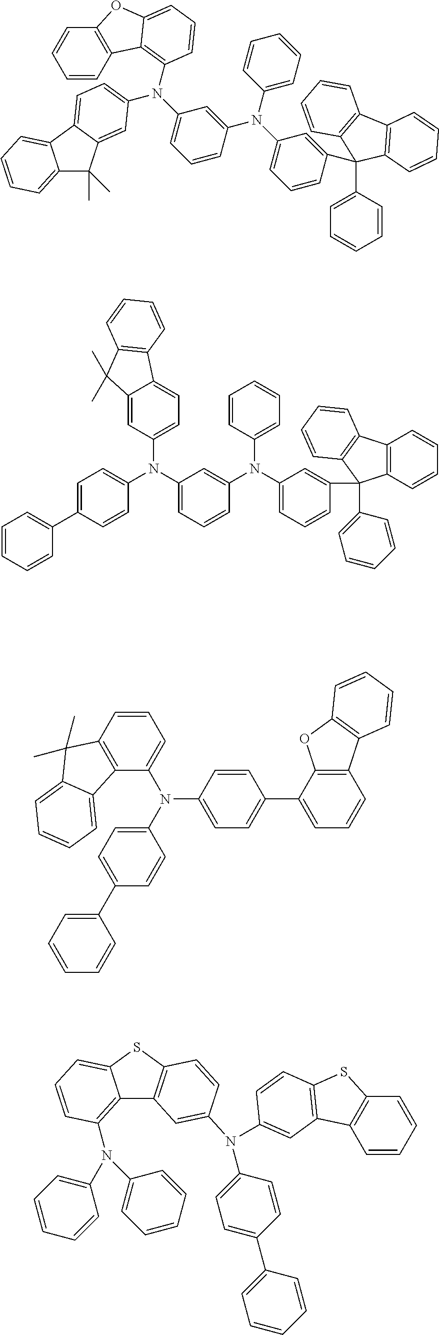 [SMILES: CC1(C)c2ccccc2-c2ccc(N(c3cccc(N(c4ccccc4)c4cccc(C5(c6ccccc6)c6ccccc6-c6ccccc65)c4)c3)c3cccc4oc5ccccc5c34)cc21 ; CC1(C)c2ccccc2-c2ccc(N(c3ccc(-c4ccccc4)cc3)c3cccc(N(c4ccccc4)c4cccc(C5(c6ccccc6)c6ccccc6-c6ccccc65)c4)c3)cc21 ; CC1(C)c2ccccc2-c2c(N(c3ccc(-c4ccccc4)cc3)c3ccc(-c4cccc5c4oc4ccccc45)cc3)cccc21 ; c1ccc(-c2ccc(N(c3ccc4sc5ccccc5c4c3)c3ccc4sc5cccc(N(c6ccccc6)c6ccccc6)c5c4c3)cc2)cc1]

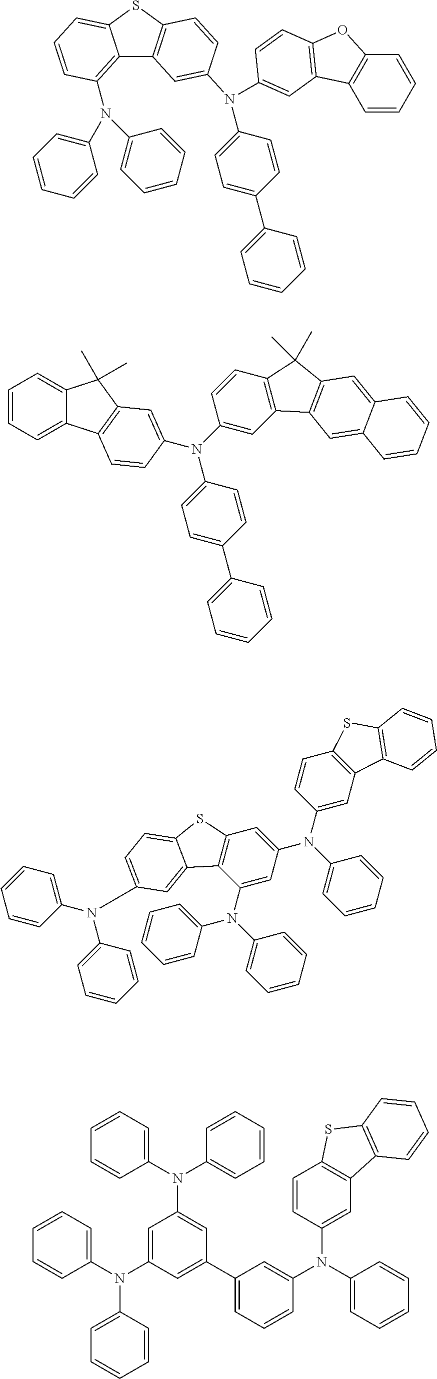

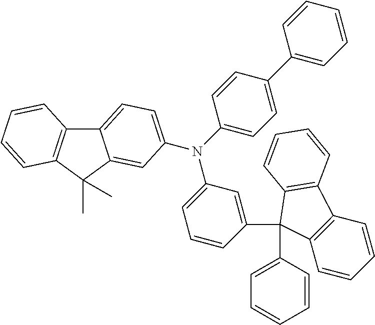 [SMILES: CC1(C)c2ccccc2-c2ccc(N(c3ccc(-c4ccccc4)cc3)c3cccc(C4(c5ccccc5)c5ccccc5-c5ccccc54)c3)cc21]

In the hole transport region 140, other suitable compounds may be used in addition to the compounds described above.

In an implementation, the charge transport region may be, e.g., the electron transport region 150.

The electron transport region 150 may further increase electron injection and/or electron mobility between the cathode 110 and the light emitting layer 130 and block holes.

In an implementation, the electron transport region 150 may include an electron transport layer between the cathode 110 and the light emitting layer 130, and an electron transport auxiliary layer between the light emitting layer 130 and the electron transport layer and a compound of Group C may be included in at least one of the electron transport layer and the electron transport auxiliary layer.

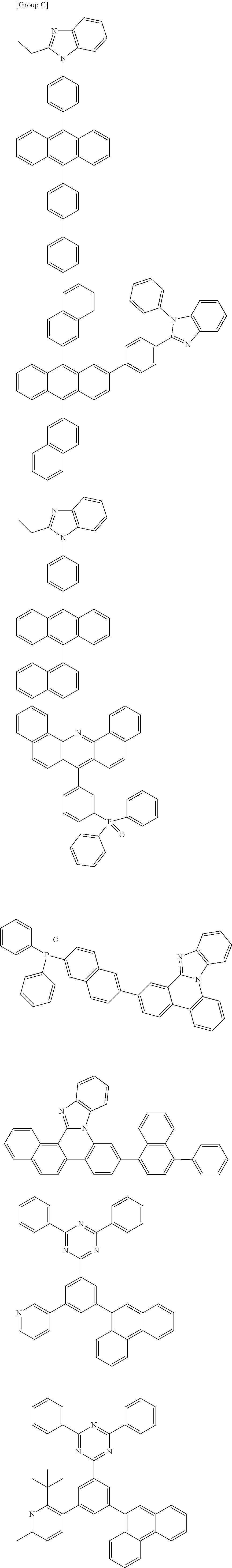

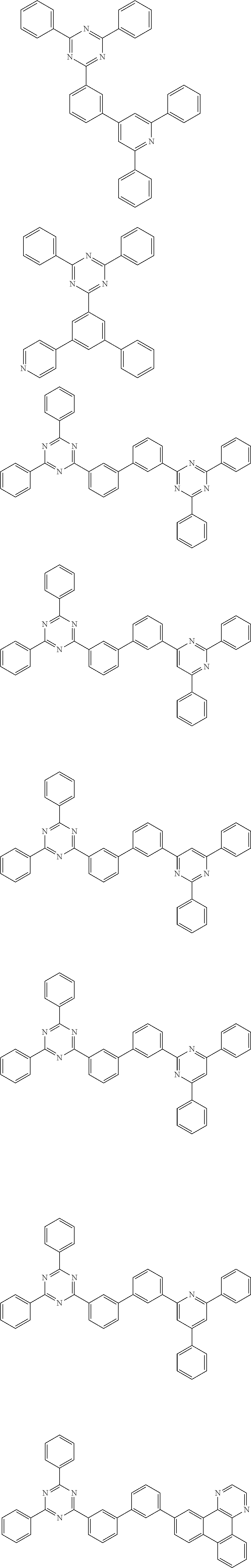

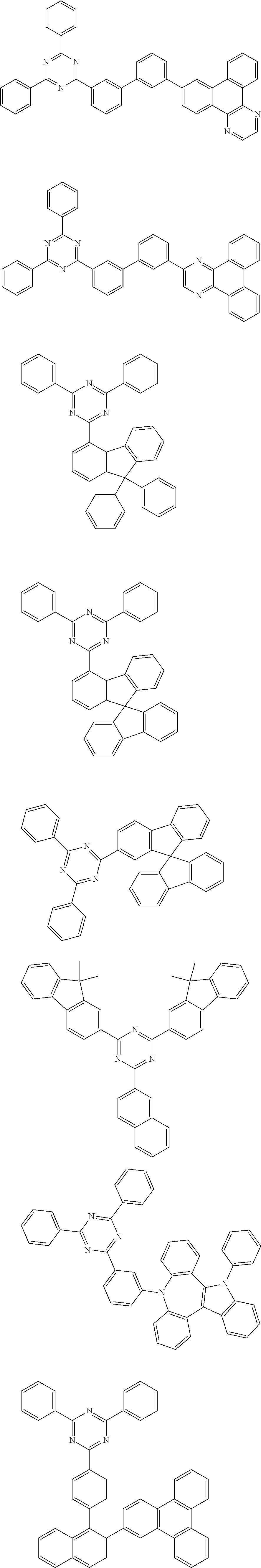

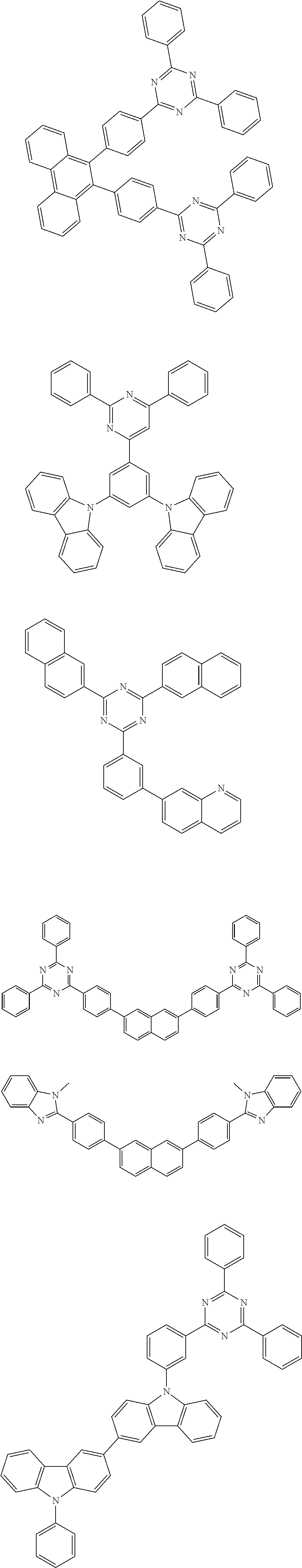

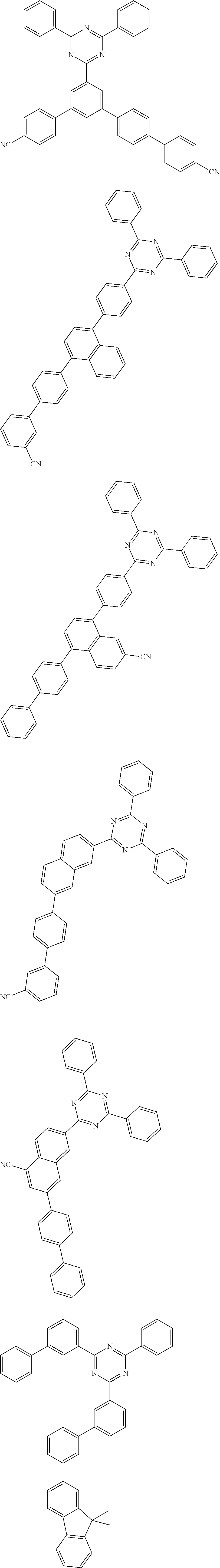

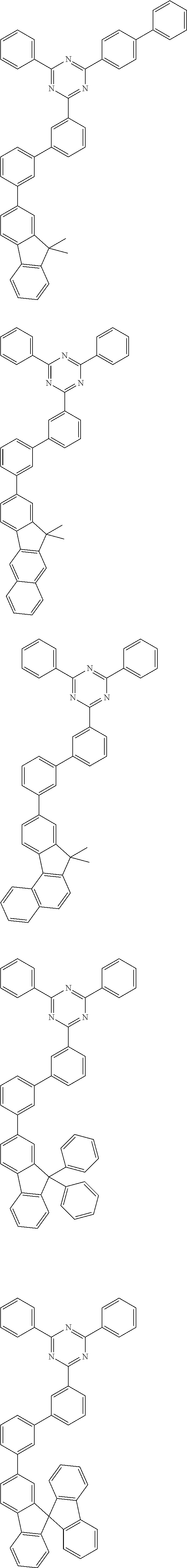

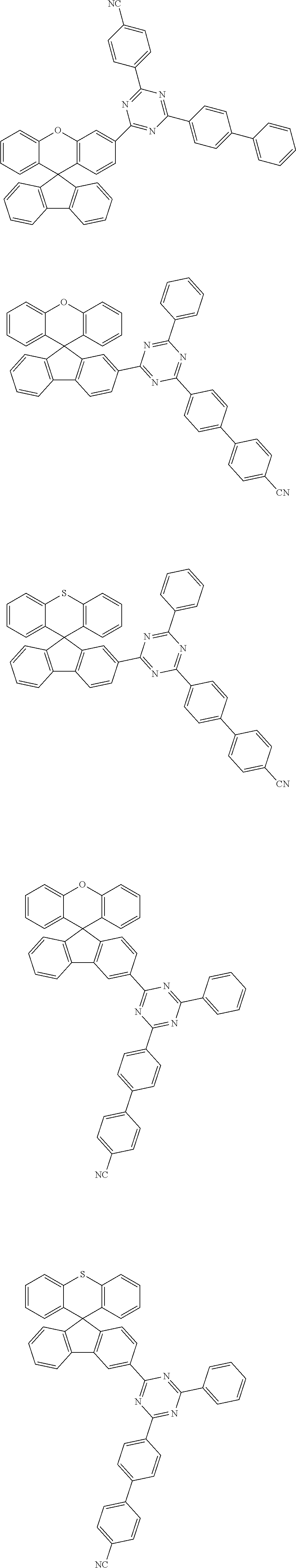 [SMILES: N#Cc1ccc(-c2nc(-c3ccc(-c4ccccc4)cc3)nc(-c3ccc4c(c3)Oc3ccccc3C43c4ccccc4-c4ccccc43)n2)cc1 ; N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1 ; N#Cc1ccc(-c2ccc(-c3nc(-c4ccccc4)nc(-c4ccc5c(c4)-c4ccccc4C54c5ccccc5Oc5ccccc54)n3)cc2)cc1 ; N#Cc1ccc(-c2ccc(-c3nc(-c4ccccc4)nc(-c4ccc5c(c4)-c4ccccc4C54c5ccccc5Sc5ccccc54)n3)cc2)cc1]

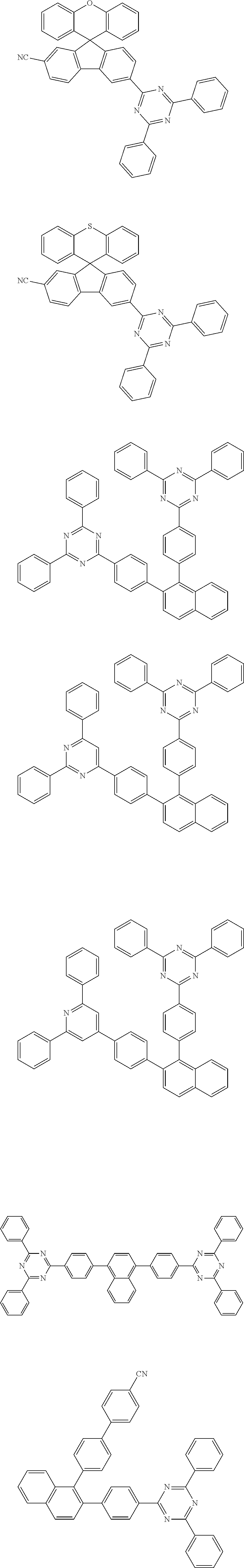

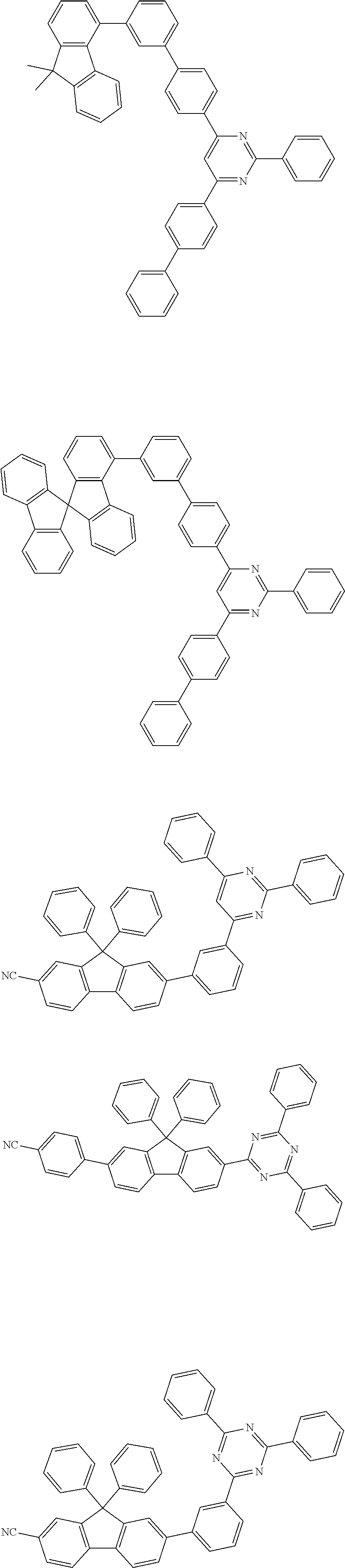 [SMILES: CC1(C)c2ccccc2-c2c(-c3cccc(-c4ccc(-c5cc(-c6ccc(-c7ccccc7)cc6)nc(-c6ccccc6)n5)cc4)c3)cccc21 ; c1ccc(-c2ccc(-c3cc(-c4ccc(-c5cccc(-c6cccc7c6-c6ccccc6C76c7ccccc7-c7ccccc76)c5)cc4)nc(-c4ccccc4)n3)cc2)cc1 ; N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment may provide an organic light emitting diode including a light emitting layer as an organic layer.

The light emitting layer may include the aforementioned composition for an organic optoelectronic device.

Another embodiment may provide an organic light emitting diode including a light emitting layer and a hole transport region as an organic layer.

The hole transport region may include the aforementioned compound for an organic optoelectronic device.

In an implementation, the hole transport auxiliary layer may include the aforementioned compound for an organic optoelectronic device.

Another embodiment may provide an organic light emitting diode including a light emitting layer and an electron transport region as an organic layer.

The organic light emitting diode according to an embodiment may include a hole transport region 140 and an electron transport region 150 in addition to the light emitting layer 130 as the organic layer 105, as shown in FIGURE.

In an implementation, the organic light emitting diode may further include an electron injection layer, a hole injection layer, or the like, in addition to the light emitting layer as the aforementioned organic layer.

The organic light emitting diode 100 may be produced by forming an anode or a cathode on a substrate, forming an organic layer using a dry film formation method, e.g., a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in examples and synthesis examples were purchased from Sigma-Aldrich Co. Ltd., TCI Inc., or Tokyo Chemical Industry as far as there is no particular comment or were synthesized by suitable methods.

(Preparation of Compound for Organic Optoelectronic Device)

### Synthesis Example 1: Synthesis of Compound 1-32

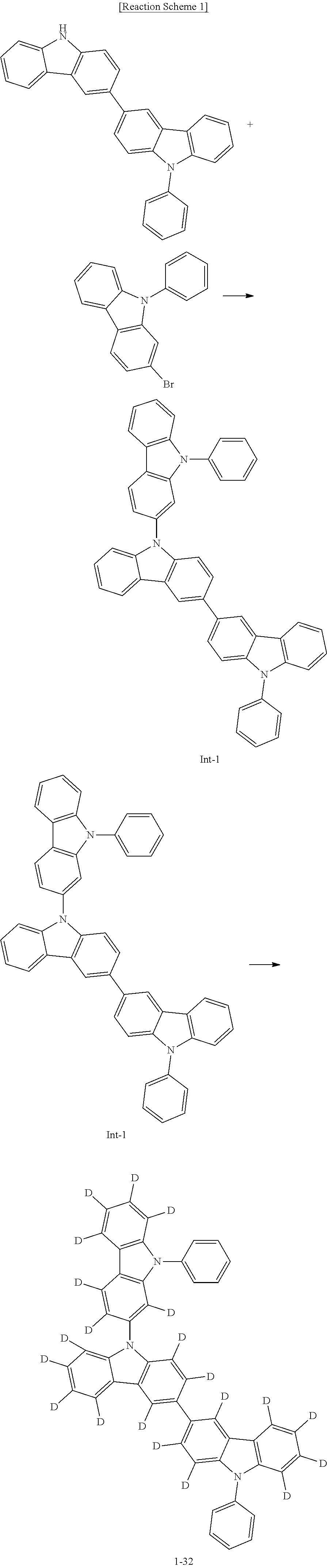

1st Step: Synthesis of Intermediate Int-1

9-phenyl-3,3′-bi-9H-carbazole (20 g, 49.0 mmol), 2-bromo-9-phenylcarbazole (15.8 g, 49.0 mmol), NaOtBu (7.1 g, 73.5 mmol), Pd₂(dba)₃ (2.2 g, 2.5 mmol), and P(t-Bu)₃ (1.5 g, 7.4 mmol) were put in a round-bottomed flask under a nitrogen atmosphere and dissolved in xylene (245 ml) and then, stirred under reflux at 120° C. for 12 hours. When a reaction was completed, an excess of distilled water is poured thereinto and then, stirred for 1 hour. A solid therein was filtered and dissolved in toluene at a high temperature. After removing moisture with MgSO₄ and filtering an organic solvent with silica gel pad, a filtrate therefrom was stirred. When a solid was formed, the solid was filtered and vacuum-dried to obtain 21.6 g (68%) of Intermediate Int-1.

2nd Step: Synthesis of Compound 1-32

Under a nitrogen condition, Intermediate Int-1 (21.6 g, 33.27 mmol), triflic acid (24.96 g, 166.35 mmol), and benzene-D₆ (174.97 g, 2079.32 mmol) were put in a round-bottomed flask and then, stirred under reflux at 50° C. for 20 hours. When a reaction was completed, D₂O (124.82 ml) was slowly poured thereinto for quenching and then, sufficiently stirred. A K₃PO₄ (aq) saturated solution was titrated to neutralize the resultant. After completing a reaction and removing an aqueous layer with a separatory funnel, an organic solvent was removed therefrom under a reduced pressure to obtain a solid. The obtained solid was dissolved in toluene at a high temperature. After removing moisture with MgSO₄ and filtering an organic solvent with silica gel pad, a filtrate therefrom was stirred. When a solid was formed, the solid was filtered and vacuum-dried to obtain 16.73 g (75%) of Compound 1-32.

### Synthesis Example 2: Synthesis of Compound 1-62

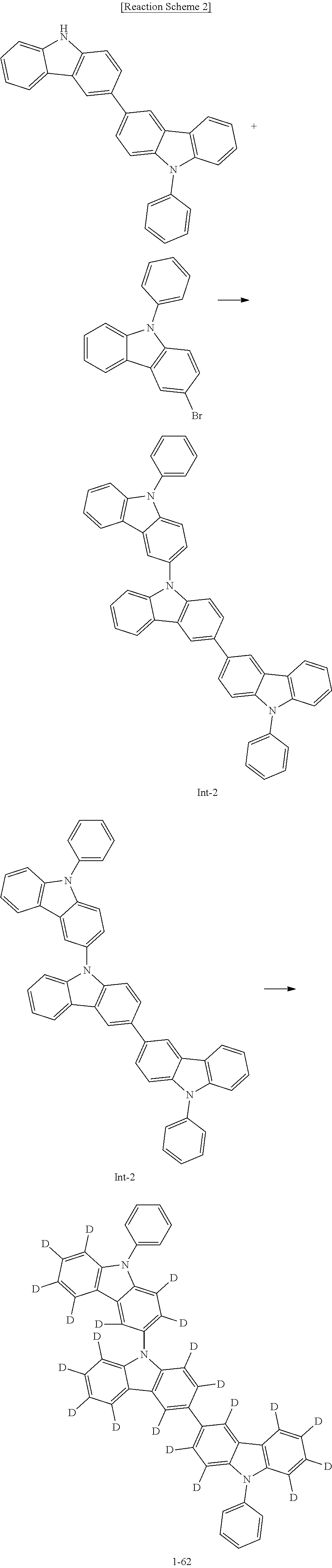

Intermediate Int-2 (22.6 g, 71%) was obtained in the same manner as in the 1^{st} step of Synthesis Example 1 except that the reactant was changed from the 2-bromo-9-phenylcarbazole to 3-bromo-9-phenylcarbazole.

2nd Step: Synthesis of Compound 1-62

Compound 1-62 (16.6 g, 71%) was obtained in the same manner as in the 2nd step of Synthesis Example 1 except that Intermediate Int-2 was used instead of Intermediate Int-1.

### Synthesis Example 3: Synthesis of Compound 1-76

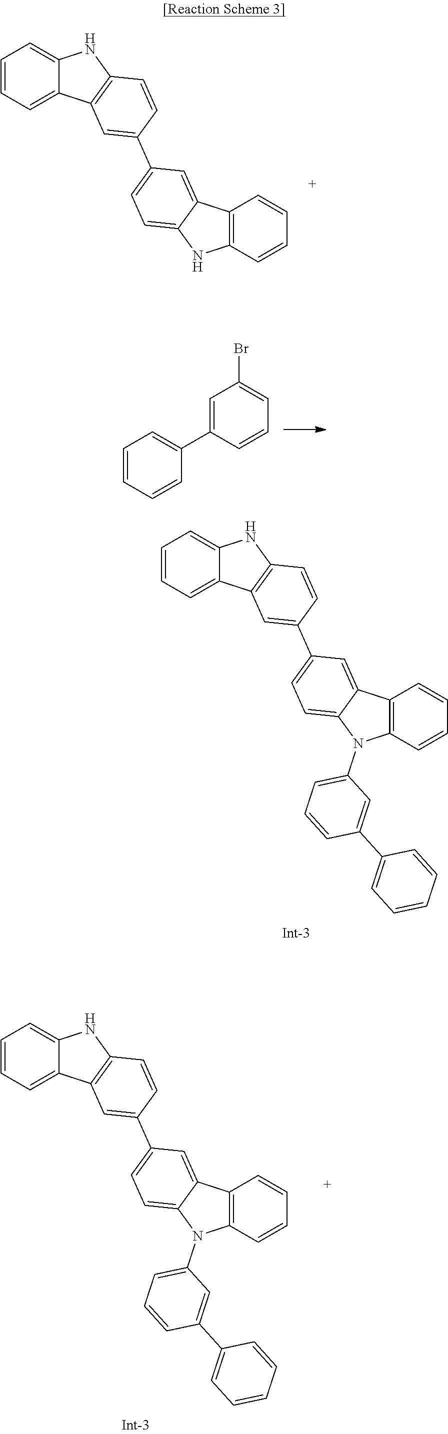 [SMILES: c1ccc2c(c1)[nH]c1ccc(-c3ccc4[nH]c5ccccc5c4c3)cc12 ; Brc1cccc(-c2ccccc2)c1 ; c1ccc(-c2cccc(-n3c4ccccc4c4cc(-c5ccc6[nH]c7ccccc7c6c5)ccc43)c2)cc1 ; c1ccc(-c2cccc(-n3c4ccccc4c4cc(-c5ccc6[nH]c7ccccc7c6c5)ccc43)c2)cc1]

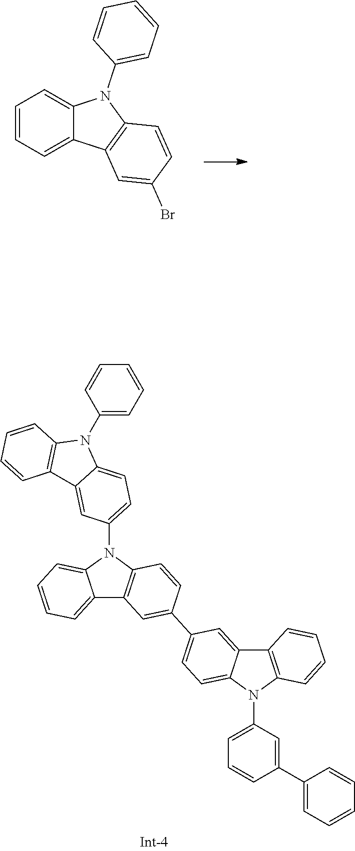 [SMILES: Brc1ccc2c(c1)c1ccccc1n2-c1ccccc1 ; c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1]

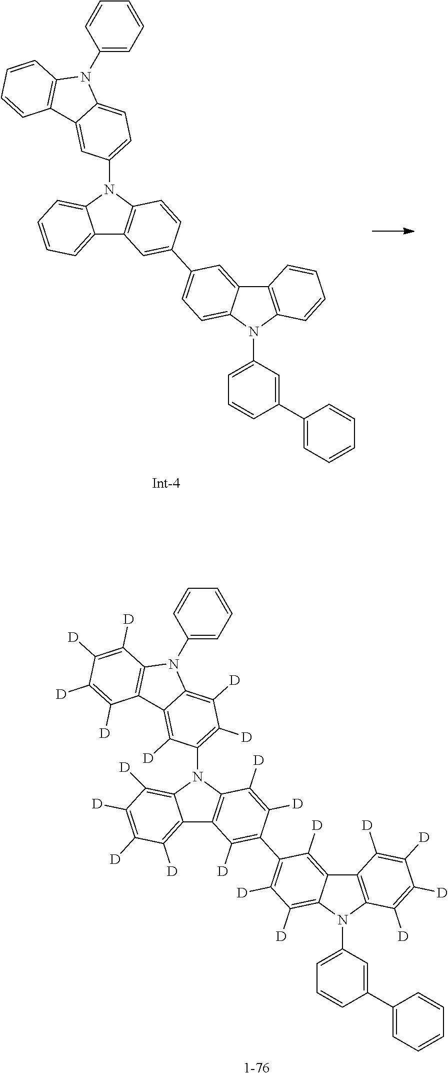 [SMILES: c1ccc(-c2cccc(-n3c4ccccc4c4cc(-c5ccc6c(c5)c5ccccc5n6-c5ccc6c(c5)c5ccccc5n6-c5ccccc5)ccc43)c2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3c([2H])c([2H])c4c(c3[2H])c3c([2H])c([2H])c([2H])c([2H])c3n4-c3ccccc3)c([2H])c([2H])c1n2-c1cccc(-c2ccccc2)c1]

1st Step: Synthesis of Intermediate Int-3

Under a nitrogen atmosphere, 3,3′biscarbazole (20 g, 60.2 mmol), 3-bromobiphenyl (7 g, 30.1 mmol), NaOtBu (8.7 g, 90.3 mmol), Pd₂(dba)₃ (2.8 g, 3.0 mmol), and P(t-Bu)₃ (1.8 g, 9.0 mmol) were put in a round-bottomed flask and dissolved in 300 ml of xylene and then, stirred under reflux at 120° C. for 12 hours. When a reaction was completed, after pouring an excess of distilled water thereinto and then, stirring the mixture for 1 hour, an aqueous layer was removed therefrom. Subsequently, 11.1 g (38%) of Intermediate Int-3 was obtained by using column chromatography (hexane:DCM (20%)).

2nd Step: Synthesis of Intermediate Int-4

Intermediate Int-4 (6.5 g, 59%) was obtained in the same manner as in the 1^{st} step of Synthesis Example 1 except that the reactants were changed from the 9-phenyl-3,3′-bi-9H-carbazole to Intermediate Int-3 and from the 2-bromo-9-phenylcarbazole to 3-bromo-9-phenylcarbazole.

3rd Step: Synthesis of Compound 1-76

Compound 1-76 (3.5 g, 52%) was obtained in the same manner as in the 2nd step of Synthesis Example 1 except that Intermediate Int-4 was used instead of Intermediate Int-1.

### Synthesis Example 4: Synthesis of Compound 1-33

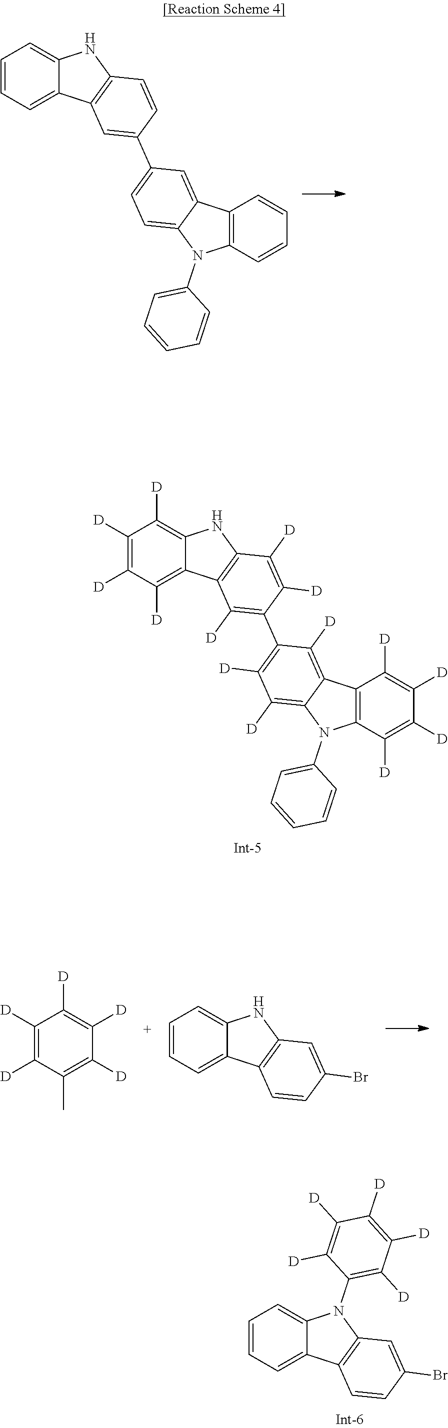 [SMILES: c1ccc(-n2c3ccccc3c3cc(-c4ccc5[nH]c6ccccc6c5c4)ccc32)cc1 ; [2H]c1c([2H])c([2H])c2c([nH]c3c([2H])c([2H])c(-c4c([2H])c([2H])c5c(c4[2H])c4c([2H])c([2H])c([2H])c([2H])c4n5-c4ccccc4)c([2H])c32)c1[2H] ; [2H]c1c([2H])c([2H])c(C)c([2H])c1[2H] ; Brc1ccc2c(c1)[nH]c1ccccc12 ; [2H]c1c([2H])c([2H])c(-n2c3ccccc3c3ccc(Br)cc32)c([2H])c1[2H]]

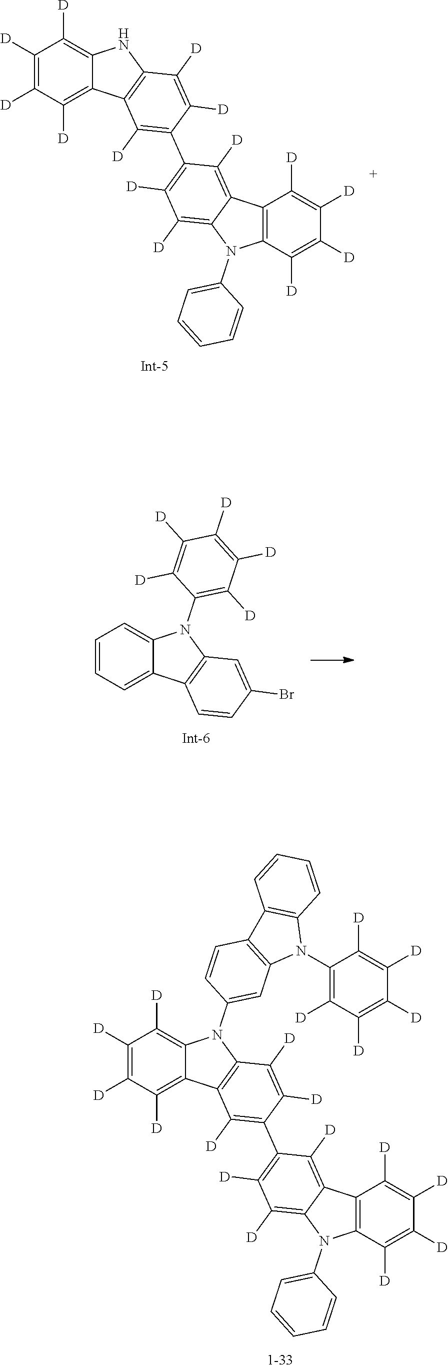 [SMILES: [2H]c1c([2H])c([2H])c2c([nH]c3c([2H])c([2H])c(-c4c([2H])c([2H])c5c(c4[2H])c4c([2H])c([2H])c([2H])c([2H])c4n5-c4ccccc4)c([2H])c32)c1[2H] ; [2H]c1c([2H])c([2H])c(-n2c3ccccc3c3ccc(Br)cc32)c([2H])c1[2H] ; [2H]c1c([2H])c([2H])c(-n2c3ccccc3c3ccc(-n4c5c([2H])c([2H])c([2H])c([2H])c5c5c([2H])c(-c6c([2H])c([2H])c7c(c6[2H])c6c([2H])c([2H])c([2H])c([2H])c6n7-c6ccccc6)c([2H])c([2H])c54)cc32)c([2H])c1[2H]]

1st Step: Synthesis of Intermediate Int-5

Under a nitrogen atmosphere, 9-phenyl-3,3′-bi-9H-carbazole (25 g, 61.2 mmol), triflic acid (45.9 g, 306.01 mmol), and benzene-D₆ (321.88 g, 3825.06 mmol) were put in a round-bottomed flask and stirred under reflux at 50° C. for 20 hours. When a reaction was completed, D₂O (229.61 ml) was slowly poured thereinto for quenching and then, sufficiently stirred. A K₃PO₄ (aq) saturated solution was titrated to neutralize the resultant. After completing a reaction and removing an aqueous layer with a separatory funnel, an organic solvent was treated through column chromatography (hexane:DCM (20%)), obtaining 15.5 g (60%) of Intermediate Int-5

2nd Step: Synthesis of Intermediate Int-6

Under a nitrogen atmosphere, iodobenzene-Ds (15 g, 71.8 mmol), 2-bromo-9H-carbazole (17.7 g, 71.8 mmol), NaOtBu (10.3 g, 107.6 mmol), Pd₂(dba)₃ (3.3 g, 3.6 mmol), and P(t-Bu)₃ (2.2 g, 10.8 mmol) were put in a round-bottomed flask and dissolved in 360 ml of xylene and then, stirred under reflux at 120° C. for 12 hours. When a reaction was completed, after pouring an excess of distilled water thereinto and then, stirring the mixture for 1 hour, an aqueous layer was removed therefrom. Subsequently, 16.4 g (70%) of Intermediate Int-6 was obtained through column chromatography (hexane:DCM (20%)).

3rd Step: Synthesis of Compound 1-33

Compound 1-33 (17.4 g, 71%) was obtained in the same manner as in the 1^{st} step of Synthesis Example 1 except that Intermediate Int-5 instead of the 9-phenyl-3,3′-bi-9H-carbazole and Intermediate Int-6 instead of the 2-bromo-9H-Carbazole were used.

### Synthesis Example 5: Synthesis of Compound 2-69

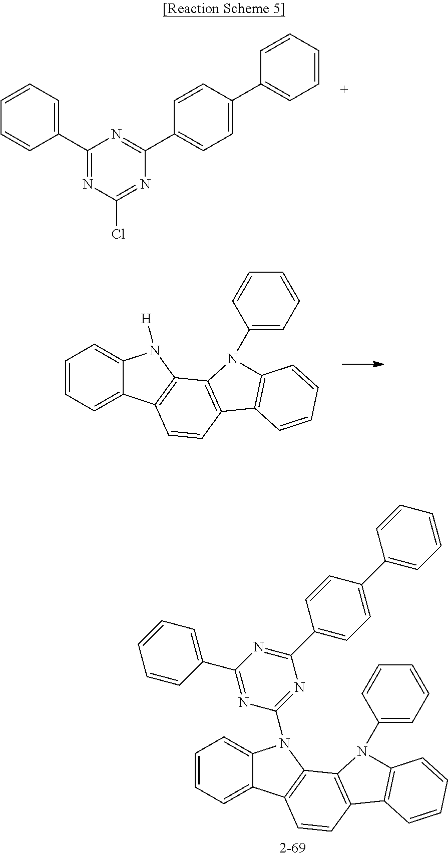 [SMILES: Clc1nc(-c2ccccc2)nc(-c2ccc(-c3ccccc3)cc2)n1 ; c1ccc(-n2c3ccccc3c3ccc4c5ccccc5[nH]c4c32)cc1 ; c1ccc(-c2ccc(-c3nc(-c4ccccc4)nc(-n4c5ccccc5c5ccc6c7ccccc7n(-c7ccccc7)c6c54)n3)cc2)cc1]

Under a nitrogen atmosphere, 2-([1,1′-biphenyl]-4-yl)-4-chloro-6-phenyl-1,3,5-triazine (15 g, 43.6 mmol), 11,12-dihydro-11-phenylindolo[2,3-a]carbazole (11.6 g, 34.9 mmol), and NaH (1.6 g, 65.4 mmol) were put in a round-bottomed flask and dissolved in 220 ml of DMF and then, stirred at room temperature for 12 hours. When a reaction was completed, an excess of distilled water was poured thereinto and then, stirred for 1 hour. A solid therein was filtered and dissolved in monochlorobenzene (MCB) at a high temperature. After removing moisture with MgSO₄ and filtering an organic solvent with silica gel pad, a filtrate therefrom was stirred. When a solid was formed, the solid was filtered and vacuum-dried, obtaining 20.4 g (73%) of Compound 2-69.

### Synthesis Example 6: Synthesis of Compound 3

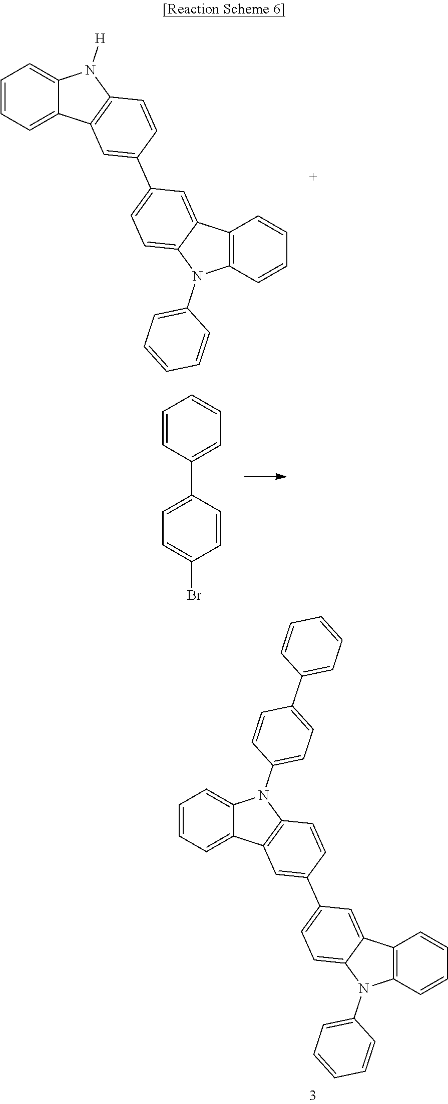

7.6 g (42%) of Compound 3 was synthesized in the same manner as in the 1^{se}t step of Synthesis Example 1 except that 4-bromo-1,1′-biphenyl was used instead of the 2-bromo-9H-carbazole and then, purified through column chromatography (hexane:DCM (20%)).

### Comparative Synthesis Example 1: Synthesis of Compound C-1

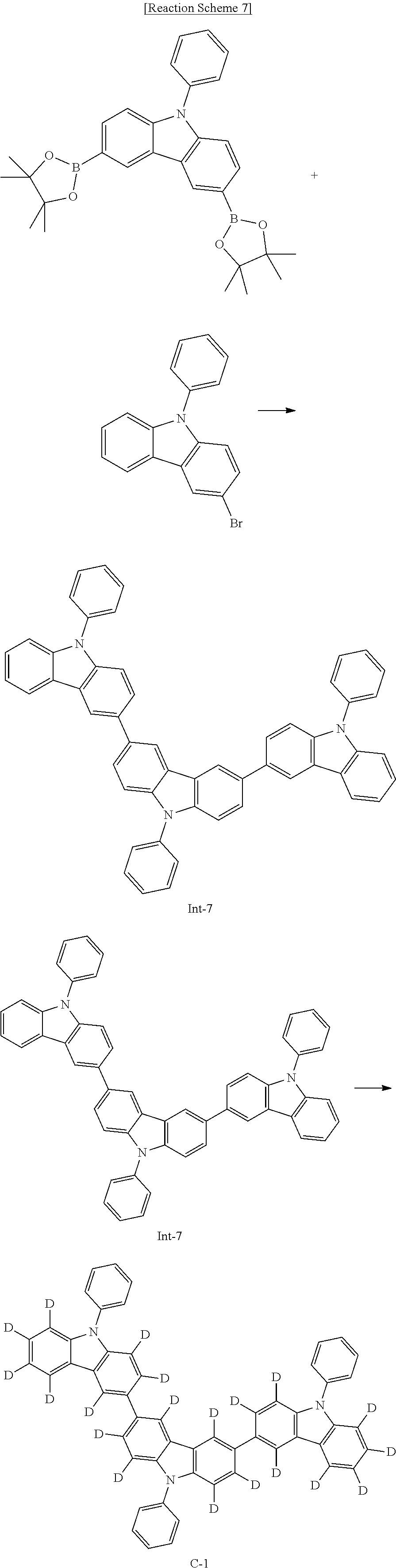

1st Step: Synthesis of Intermediate Int-7

Under a nitrogen atmosphere, 9-phenyl-3,6-bis (4,4,5,5-tetramethyl-1,3,2-dioxaborolan-2-yl)-9H-carbazole (20 g, 40.4 mmol), 3-bromo-9-phenylcarbazole (32.5 g, 101 mmol), K₂CO₃ (16.7 g, 121.2 mmol), and Pd(PPh₃)₄ (2.3 g, 2 mmol) were put in a round-bottomed flask and dissolved in 135 ml of THE and 70 ml of distilled water and then, stirred under reflux at 60° C. for 12 hours. When a reaction was completed, after removing an aqueous layer, an organic solvent was removed therefrom under a reduced pressure, obtaining a solid. The solid was dissolved in MCB at a high temperature.

After removing moisture with MgSO₄ and filtering an organic solvent with silica gel pad, a filtrate therefrom was stirred. When a solid was formed, the solid was filtered and vacuum-dried, obtaining 18.2 g (62%) of Intermediate Int-7.

2nd Step: Synthesis of Compound C-1

Compound C-1 (13.3 g, 71%) was obtained in the same manner as in the 2nd step of Synthesis Example 1 except that Intermediate Int-7 was used instead of Intermediate Int-1.

### Comparative Synthesis Example 2: Synthesis of Compound C-2

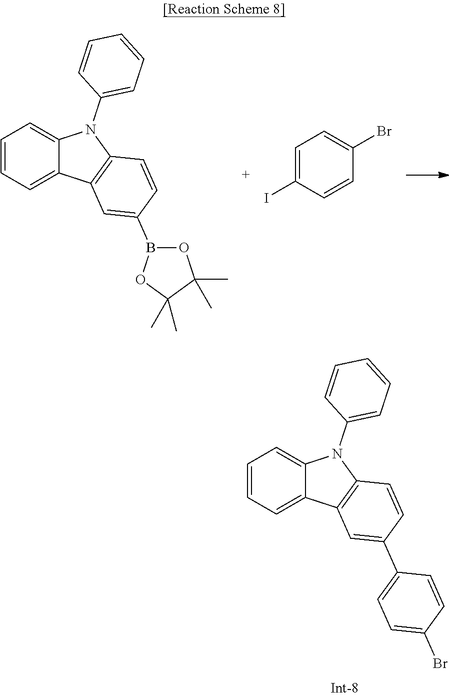

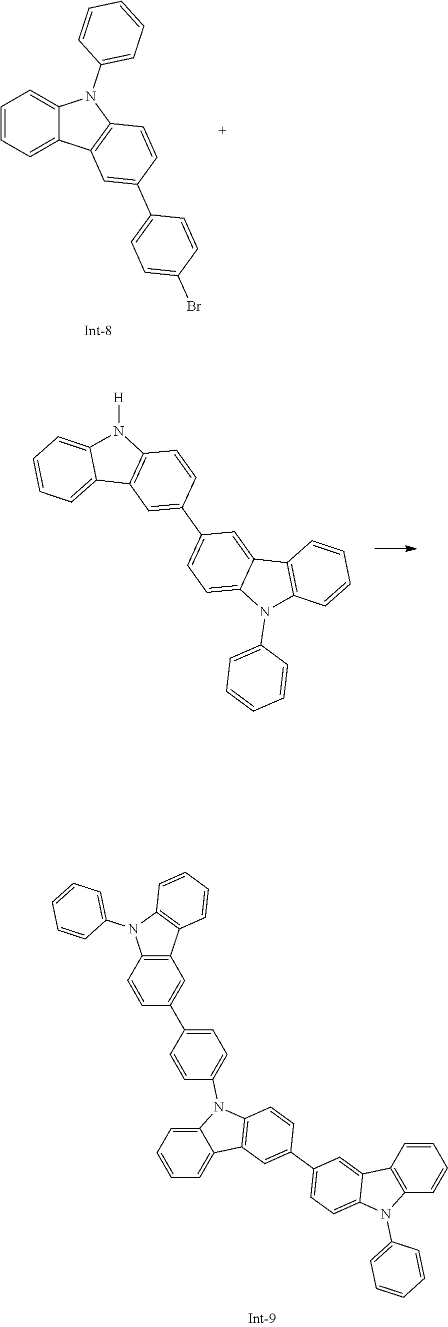 [SMILES: Brc1ccc(-c2ccc3c(c2)c2ccccc2n3-c2ccccc2)cc1 ; c1ccc(-n2c3ccccc3c3cc(-c4ccc5[nH]c6ccccc6c5c4)ccc32)cc1 ; c1ccc(-n2c3ccccc3c3cc(-c4ccc(-n5c6ccccc6c6cc(-c7ccc8c(c7)c7ccccc7n8-c7ccccc7)ccc65)cc4)ccc32)cc1]

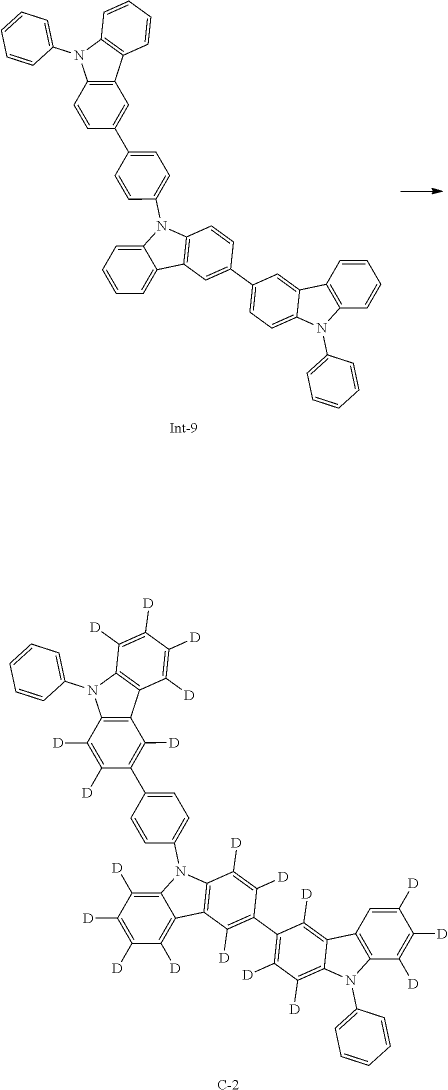

1st Step: Synthesis of Intermediate Int-8

Intermediate Int-8 (11.6 g, 54%) was obtained in the same manner as in the 1^{st} step of Comparative Synthesis Example 1 except that 9-phenyl-3-(4,4,5,5-tetramethyl-1,3,2-dioxaborolan-2-yl)carbazole instead of the 9-phenyl-3,6-bis(4,4,5,5-tetramethyl-1,3,2-dioxaborolan-2-yl)-9H-carbazole and 1-bromo-4-iodobenzene instead of the 3-bromo-9-phenylcarbazole were used.

2nd Step: Synthesis of Intermediate Int-9

Intermediate Int-9 (26.6 g, 73%) was obtained in the same manner as in the 1^{st} step of Synthesis Example 1 except that Intermediate Int-8 was used instead of the 2-bromo-9H-carbazole.

3rd Step: Synthesis of Compound C-2

Compound C-2 (20 g, 73%) was obtained in the same manner as in the 2^{nd} step of Synthesis Example 1 except that Intermediate Int-9 was used instead of Intermediate Int-1.

(Manufacture of Organic Light Emitting Diode)

### Example 1

A glass substrate coated with ITO (indium tin oxide) was ultrasonically washed with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound A doped with 1% NDP-9 (Novaled GmbH) was vacuum-deposited on the ITO substrate to form a 100 Å-thick hole injection layer, and Compound A was deposited on the hole injection layer to a thickness of 1,350 Å to form a hole transport layer. Compound 1-32 of Synthesis Example 1 was deposited on the hole transport layer to a thickness of 350 Å to form a hole transport auxiliary layer. On the hole transport auxiliary layer, a mixture of Compound 2-69 obtained in Synthesis Example 5 and Compound 3 obtained in Synthesis Example 6 in a weight ratio of 3:7 was used as a host, and was doped with 10 wt % of PhGD as a dopant to form a 400 Å-thick light emitting layer by vacuum deposition. Subsequently, Compound C was deposited to form a 50 Å-thick electron transport auxiliary layer on the light emitting layer, and Compound D and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

The structure was ITO/Compound A (1% NDP-9 doping, 100 Å)/Compound A (1,350 Å)/Compound 1-32 (350 Å)/EML[90 wt % of host (Compound 2-69:Compound 3=3:7 w/w):10 wt % of PhGD] (400 Å)/Compound C (50 Å)/Compound D:LiQ (300 Å)/LiQ (15 Å)/Al (1,200 Å).

Compound A: N-(biphenyl-4-yl)-9,9-dimethyl-N-(4-(9-phenyl-9H-carbazol-3-yl)phenyl)-9H-fluoren-2-amine

Compound B: N,N-bis(9,9-dimethyl-9H-fluoren-4-yl)-9,9-spirobi(fluorene)-2-amine

Compound C: 2-(3-(3-(9,9-dimethyl-9H-fluoren-2-yl)phenyl)phenyl)-4,6-diphenyl-1,3,5-triazine

Compound D: 2-(Biphenyl-4-yl)-4-(9,9-diphenyl-9H-fluoren-4-yl)-6-phenyl-1,3,5-triazine

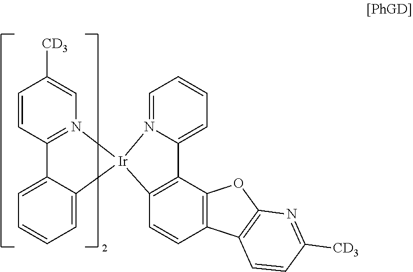

### Example 2

An organic light emitting diode was manufactured in the same manner as Example 1 except that Compound 1-62 of Synthesis Example 2 was used instead of Compound 1-32.

### Comparative Example 1

An organic light emitting diode was manufactured in the same manner as Example 1 except that Compound C-1 of Comparative Synthesis Example 1 was used instead of Compound 1-32.

### Example 3

A glass substrate coated with ITO (indium tin oxide) was ultrasonically washed with distilled water. After washing with the distilled water, the glass substrate was ultrasonically washed with isopropyl alcohol, acetone, or methanol, and dried and then, moved to a plasma cleaner, cleaned by using oxygen plasma for 10 minutes, and moved to a vacuum depositor. This prepared ITO transparent electrode was used as an anode, Compound A doped with 1% NDP-9 (Novaled GmbH) was vacuum-deposited on the ITO substrate to form a 100 Å-thick hole injection layer, and Compound A was deposited on the hole injection layer to a thickness of 1,350 Å to form a hole transport layer. Compound B was deposited to be a thickness of 350 Å on the hole transport layer to form a hole transport auxiliary layer. On the hole transport auxiliary layer, a mixture of Compound 1-32 obtained in Synthesis Example 1 and Compound 2-69 obtained in Synthesis Example 5 in a weight ratio of 7:3 was used as a host, and was doped with 10 wt % of PhGD as a dopant to form a 400 Å-thick light emitting layer by vacuum deposition. Subsequently, Compound C was deposited to form a 50 Å-thick electron transport auxiliary layer on the light emitting layer, and Compound D and LiQ were simultaneously vacuum-deposited in a weight ratio of 1:1 to form a 300 Å-thick electron transport layer. On the electron transport layer, LiQ and Al were sequentially vacuum-deposited to be 15 Å-thick and 1,200 Å-thick, manufacturing an organic light emitting diode.

The structure was ITO/Compound A (1% NDP-9 doping, 100 Å)/Compound A (1,350 Å)/Compound B (350 Å)/EML [90 wt % of host (Compound 1-32:Compound 2−69=7:3 w/w):10 wt % of PhGD] (400 Å)/Compound C (50 Å) /Compound D:LiQ (300 Å)/LiQ (15 Å)/Al (1,200 Å).

### Example 4

An organic light emitting diode was manufactured in the same manner as Example 3 except that Compound 1-62 of Synthesis Example 2 was used instead of Compound 1-32.

### Comparative Example 2

An organic light emitting diode was manufactured in the same manner as Example 3 except that Compound C-1 of Comparative Synthesis Example 1 was used instead of Compound 1-32.

### Comparative Example 3

An organic light emitting diode was manufactured in the same manner as in Example 3 except that Compound C-2 of Comparative Synthesis Example 2 was used instead of Compound 1-32.

### Comparative Example 4

An organic light emitting diode was manufactured in the same manner as in Example 3 except that Compound Int-1 of Synthesis Example 1 was used instead of Compound 1-32.

### Comparative Example 5

An organic light emitting diode was manufactured in the same manner as in Example 3 except that Compound Int-2 of Synthesis Example 2 was used instead of Compound 1-32.

Evaluation

The driving voltage and life-span characteristics of the organic light emitting diodes according to Examples 1 to 4 and Comparative Examples 1 to 5 were evaluated.

Specific measurement methods were as follows, and the results are shown in Tables 1 and 2.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value is divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Current Efficiency

Current efficiency (cd/A) at the same current density (10 mA/cm²) is calculated by using the luminance and current density from the items (1) and (2) and voltages.

(4) Driving Voltage

A driving voltage of each diode was measured by using a current-voltage meter (Keithley 2400) at 15 mA/cm².

(5) Power Efficiency

The power efficiency value was calculated from Equation 1, and the relative values based on the power efficiency of Comparative Example 1 were calculated and shown in Table 1.

Power efficiency (lm/W)=[Current efficiency (cd/A)/Driving voltage (V)]*Π [Equation 1]

(Π means the ratio of circumference)

(6) Measurement of Life-span

The luminance (cd/m²) was maintained at 24,000 cd/m², and the time at which the current efficiency (cd/A) decreased to 97% was measured to obtain results.

The relative values obtained by converting the life-span of T97 of Example 4 into 100% are shown in Table 2.

TABLE 1

| No. | Hole transport auxiliary layer | Power efficiency ratio (%) |
|---|---|---|
| Example 1 | 1-32 | 114% |
| Example 2 | 1-62 | 110% |
| Comparative Example 1 | C-1 | 100% |

Referring to Table 1, the organic light emitting diodes manufactured by using the compound represented by the above Chemical Formula 1 as a hole transport auxiliary layer material particularly realized high efficiency characteristics.

TABLE 2

| No. | First host | Second host | T97 life-span ratio (%) |
|---|---|---|---|
| Example 3 | 1-32 | 2-69 | 117% |
| Example 4 | 1-62 | 2-69 | 100% |
| Comparative Example 2 | C-1 | 2-69 | 13% |
| Comparative Example 3 | C-2 | 2-69 | 71% |
| Comparative Example 4 | Int-1 | 2-69 | 90% |
| Comparative Example 5 | Int-2 | 2-69 | 76% |

Referring to Table 2, the organic light emitting diodes manufactured by using the compound represented by the above Chemical Formula 1 as a host material of a light emitting layer particularly realized long life-span characteristics. In particular, compared with the diodes to which different compounds having the same or similar skeleton to that of Chemical Formula 1 but differing in a bonding position of carbazole and deuterium substitution or not were applied, the organic light emitting diodes to which the compound represented by the above Chemical Formula 1 was applied exhibited a clear increase in life-span.

One or more embodiments may provide a compound for an organic optoelectronic device capable of implementing an organic optoelectronic device having high efficiency and a long life-span.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. A compound for an organic optoelectronic device, the compound being represented by Chemical Formula 1: 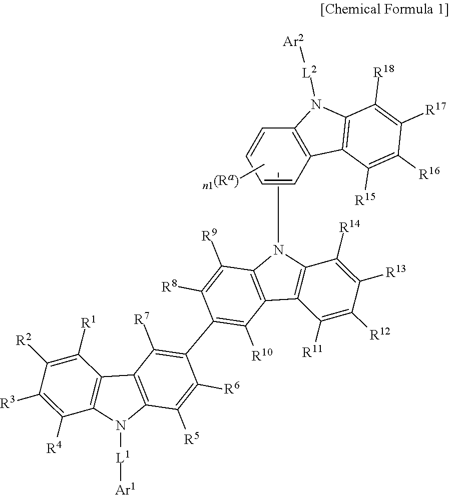
wherein, in Chemical Formula 1,
R^{a} and R¹ to R¹⁸ are each independently hydrogen, deuterium, a halogen, a substituted or unsubstituted C1 to C30 alkyl group, or a substituted or unsubstituted C6 to C30 aryl group,
n1 is an integer of 1 to 3,
Ar¹ and Ar² are each independently a substituted or unsubstituted C1 to C30 alkyl group, a substituted or unsubstituted C6 to C30 aryl group, or a substituted or unsubstituted C2 to C30 heterocyclic group, and
L¹ and L² are each independently a single bond, a substituted or unsubstituted C6 to C30 arylene group, or a substituted or unsubstituted C2 to C30 heterocyclic group,
provided that said compound meets one or more of the following:
at least one of R^{a} and R¹ to R¹⁸ is deuterium;
at least one of R^{a} and R¹ to R¹⁸ is a C1 to C30 alkyl group substituted with at least one deuterium or a C6 to C30 aryl group substituted with at least one deuterium; or
at least one of Ar¹ and Ar² is a C1 to C30 alkyl group substituted with at least one deuterium, a C6 to C30 aryl group substituted with at least one deuterium, or a C2 to C30 heterocyclic group substituted with at least one deuterium.

2. The compound for an organic optoelectronic device as claimed in claim 1, wherein:
Chemical Formula 1 is represented by one of Chemical Formula 1-I to Chemical Formula 1-IV: 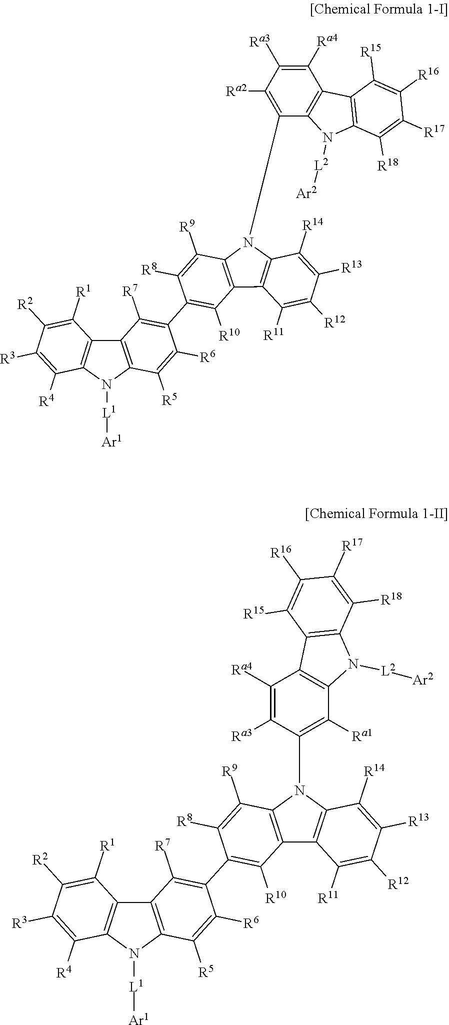 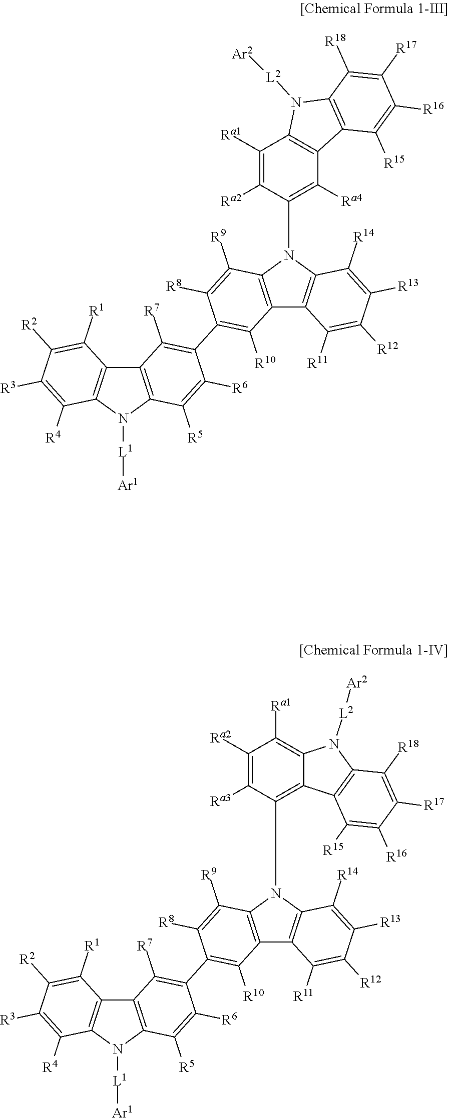
in Chemical Formula 1-I to Chemical Formula 1-IV,
R¹ to R¹⁸, Ar¹, Ar², L¹, and L² are defined the same as those of Chemical Formula 1, and
R^{a1} to R^{a4} are each independently defined the same as R^{a} of Chemical Formula 1.

3. The compound for an organic optoelectronic device as claimed in claim 1, wherein:
Chemical Formula 1 is represented by Chemical Formula 1A: 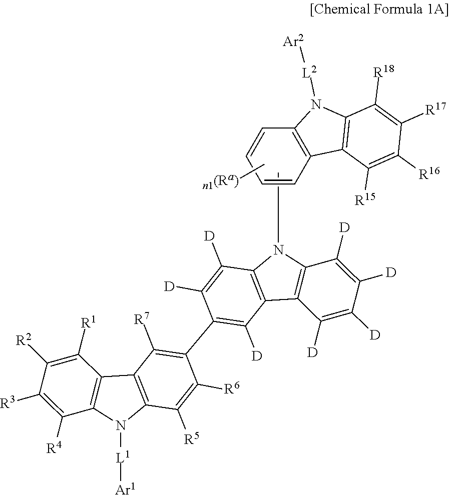
in Chemical Formula 1A,
R^{a}, R¹ to R⁷, and R¹⁵ to R¹⁸ are each independently hydrogen, deuterium, a halogen, a substituted or unsubstituted C1 to C30 alkyl group, or a substituted or unsubstituted C6 to C30 aryl group,
n1 an integer of 1 to 3,
Ar¹ and Ar² are each independently a substituted or unsubstituted C1 to C30 alkyl group or a substituted or unsubstituted C6 to C30 aryl group, and
L¹ and L² are each independently a single bond or a substituted or unsubstituted C6 to C30 arylene group.

4. The compound for an organic optoelectronic device as claimed in claim 1, wherein:
Chemical Formula 1 is represented by Chemical Formula 1B: 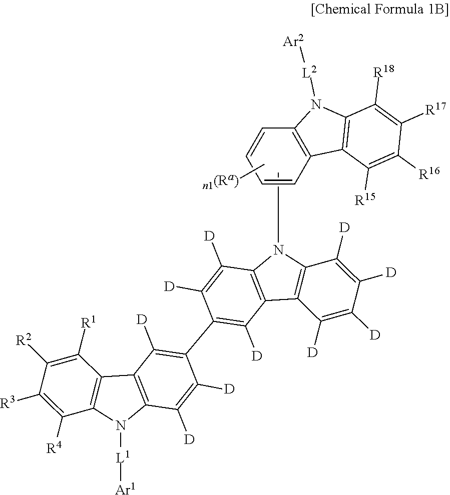
in Chemical Formula 1B,
R^{a}, R¹ to R⁴, and R¹⁸ to R¹⁸ are each independently hydrogen, deuterium, a halogen, a substituted or unsubstituted C1 to C30 alkyl group, or a substituted or unsubstituted C6 to C30 aryl group,
n1 is an integer of 1 to 3,
Ar¹ and Ar² are each independently a substituted or unsubstituted C1 to C30 alkyl group or a substituted or unsubstituted C6 to C30 aryl group, and
L¹ and L² are each independently a single bond or a substituted or unsubstituted C6 to C30 arylene group.

5. The compound for an organic optoelectronic device as claimed in claim 1, wherein:
Chemical Formula 1 is represented by Chemical Formula 1C: 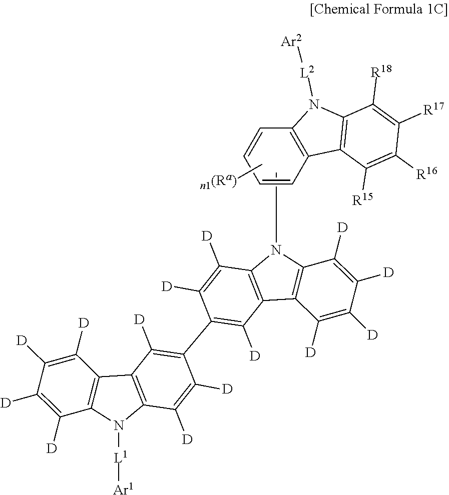
in Chemical Formula 1C,
R^{a} and R¹⁵ to R¹⁸ are each independently hydrogen, deuterium, a halogen, a substituted or unsubstituted C1 to C30 alkyl group, or a substituted or unsubstituted C6 to C30 aryl group,
n1 is an integer of 1 to 3,
Ar¹ and Ar² are each independently a substituted or unsubstituted C1 to C30 alkyl group or a substituted or unsubstituted C6 to C30 aryl group, and
L¹ and L² are each independently a single bond or a substituted or unsubstituted C6 to C30 arylene group.

6. The compound for an organic optoelectronic device as claimed in claim 1, wherein:
at least one of R^{a} and R¹ to R¹⁸ is deuterium;
at least one of R^{a} and R¹ to R¹⁸ is a C1 to C10 alkyl group substituted with at least one deuterium or a C6 to C20 aryl group substituted with at least one deuterium; or
at least one of Ar¹ and Ar² is a C1 to C10 alkyl group substituted with at least one deuterium or a C6 to C20 aryl group substituted with at least one deuterium.

7. The compound for an organic optoelectronic device as claimed in claim 6, wherein:
the C6 to C20 aryl group substituted with at least one deuterium is a group of Group I: 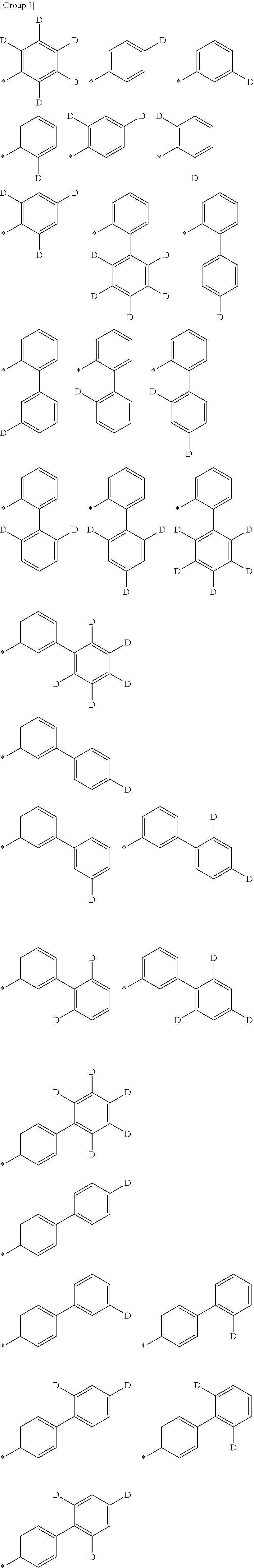
in Group I, * is a linking point.

8. The compound for an organic optoelectronic device as claimed in claim 1, wherein the compound is a compound of Group 1:

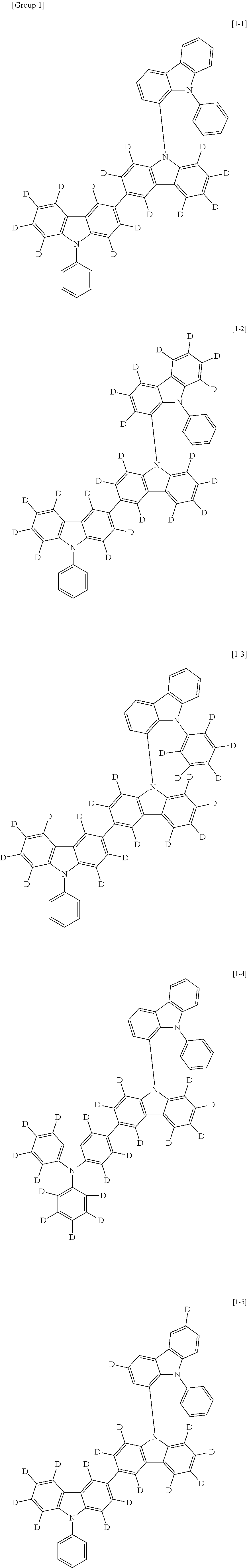

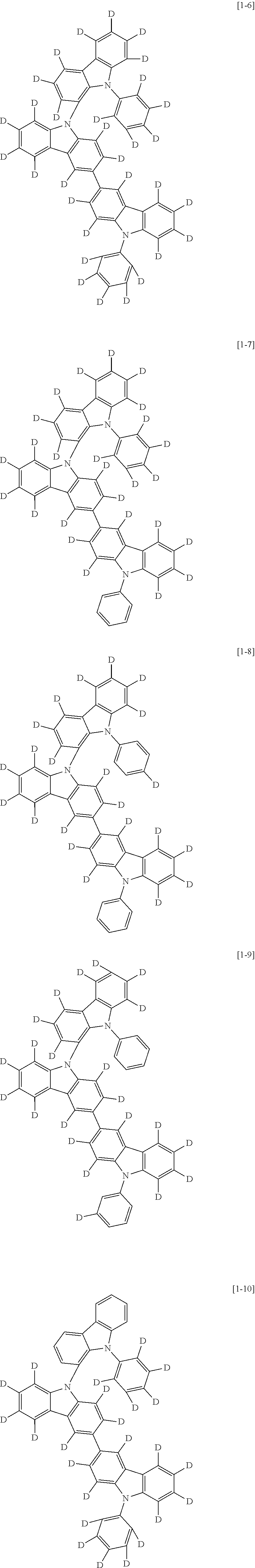

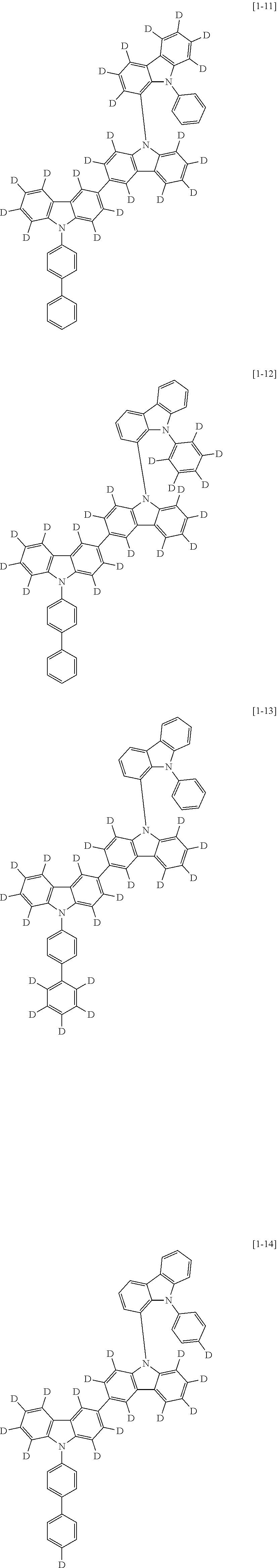

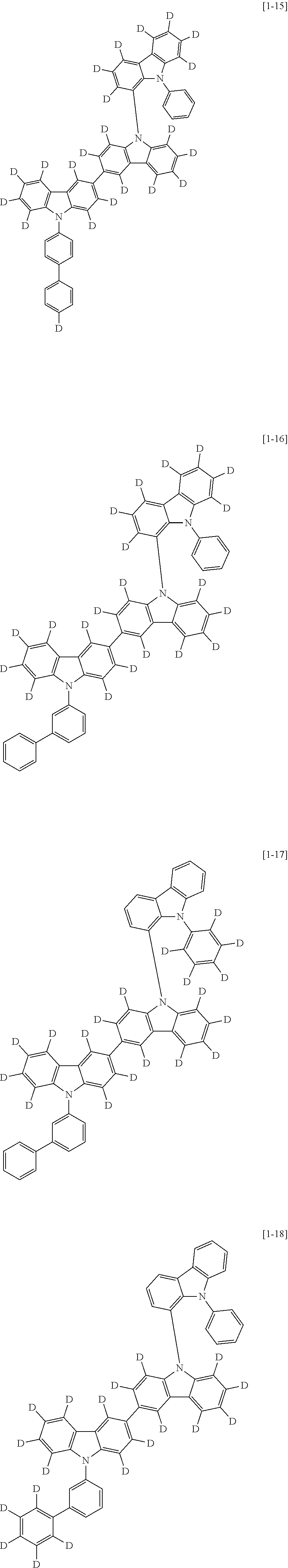

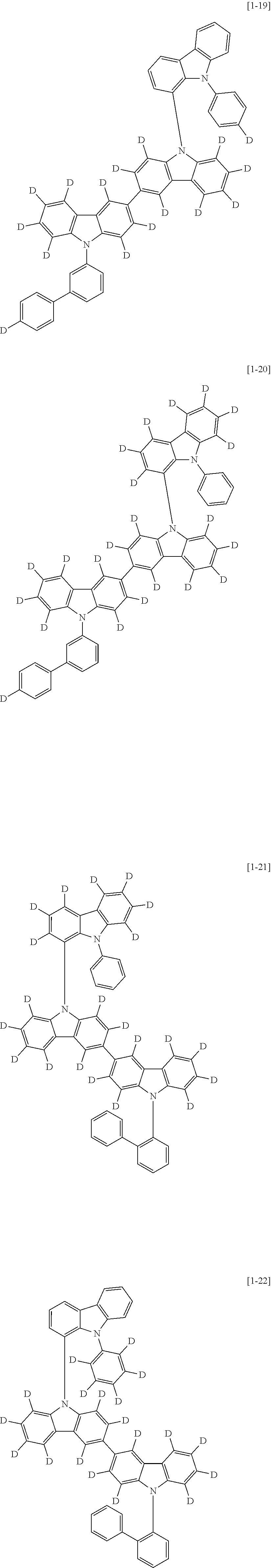

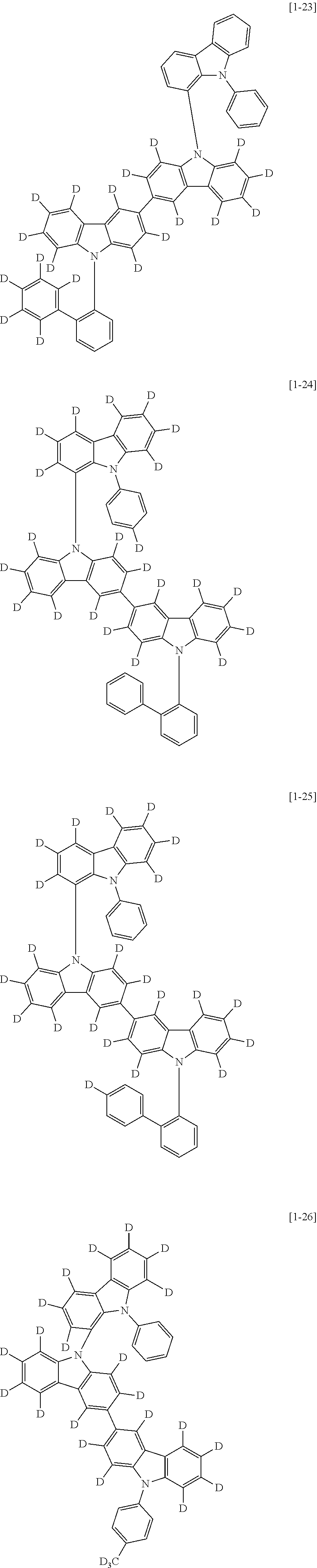

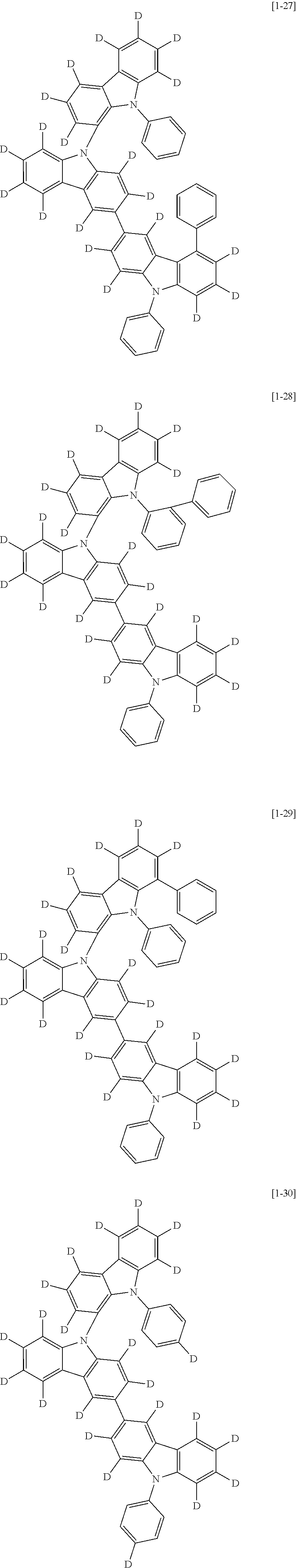

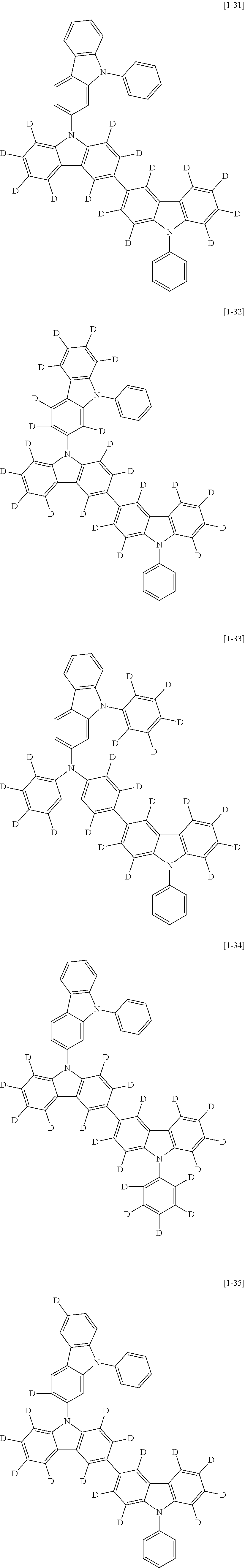

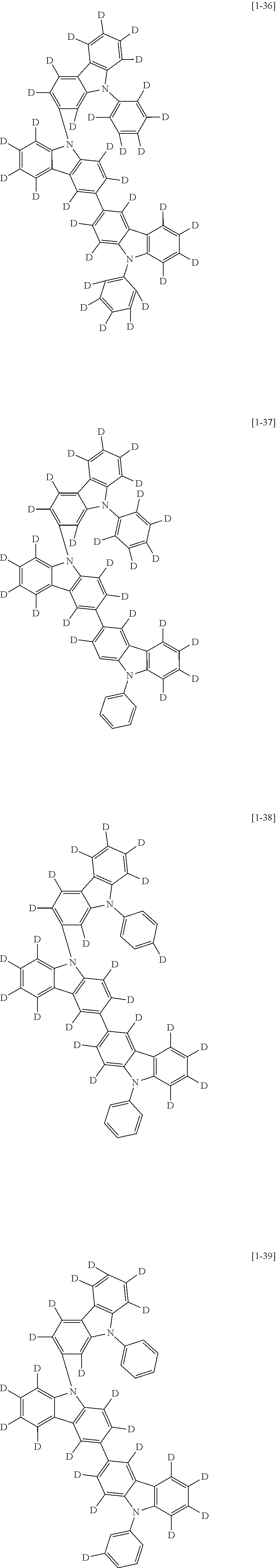

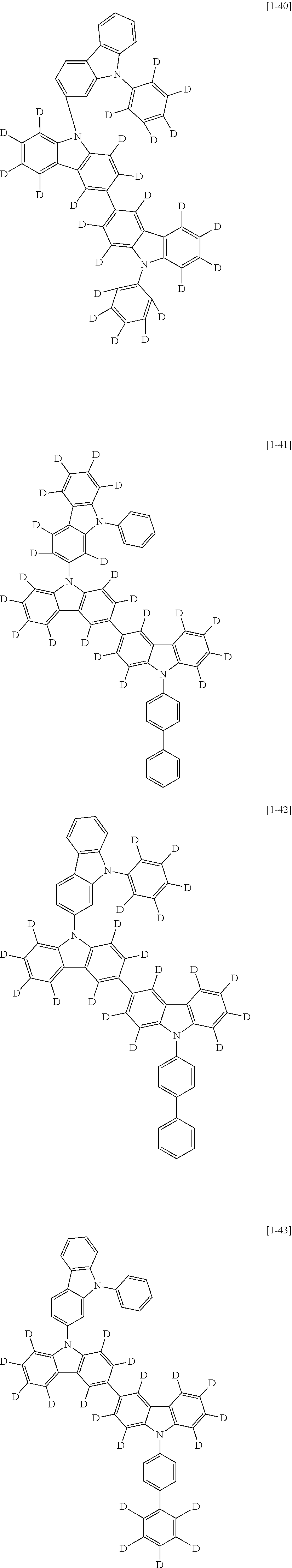

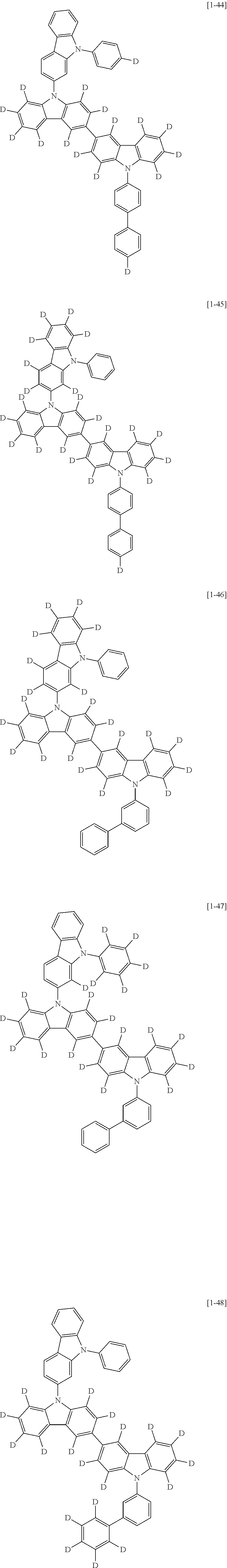

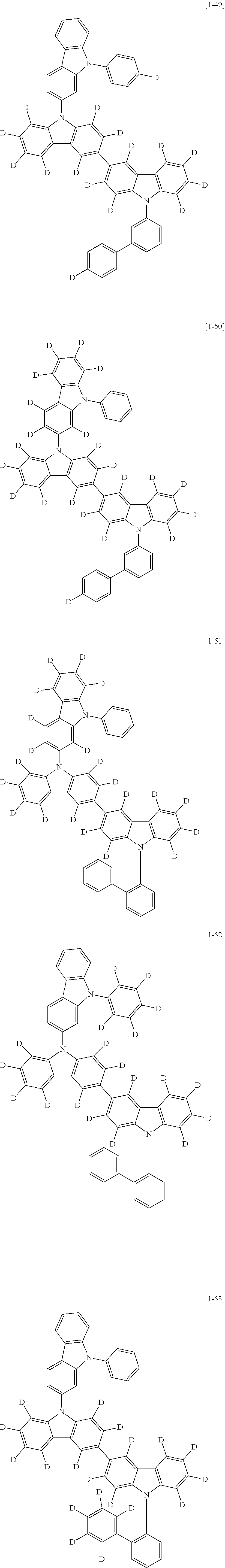

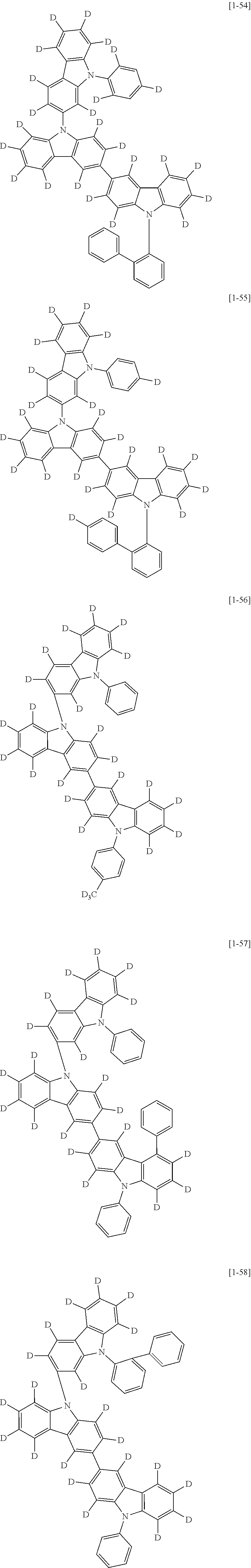

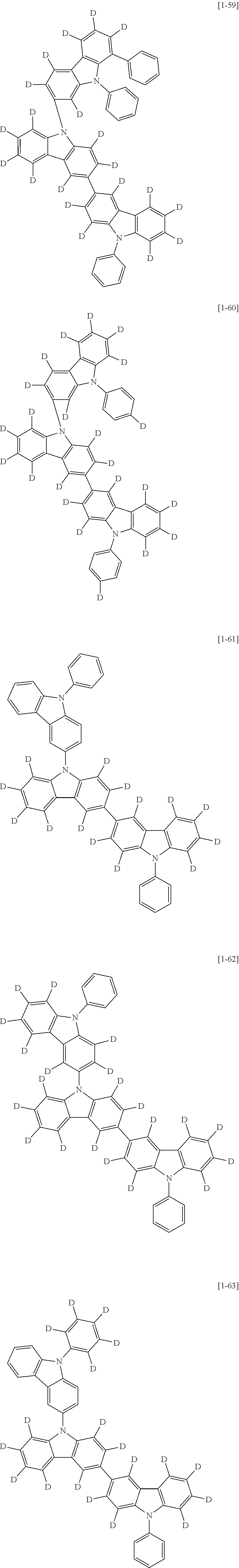

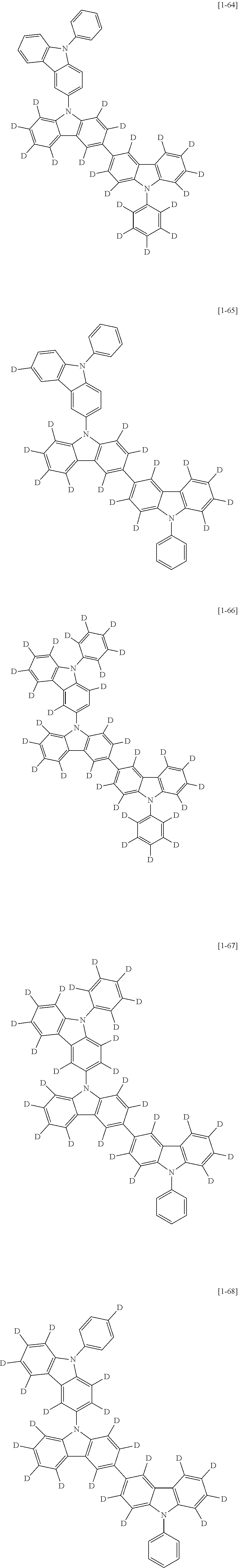

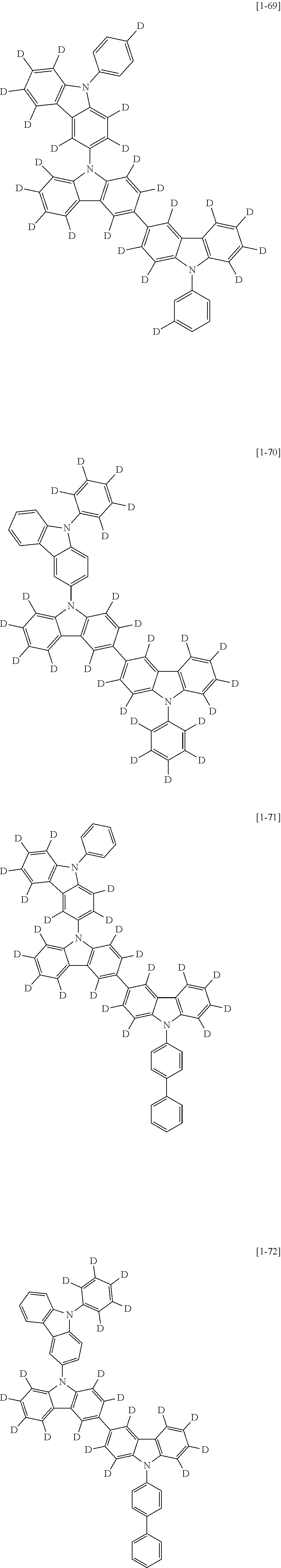

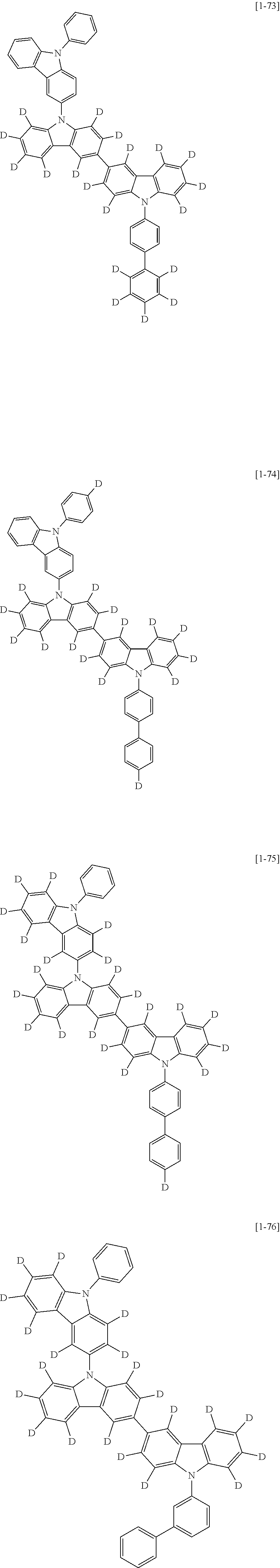

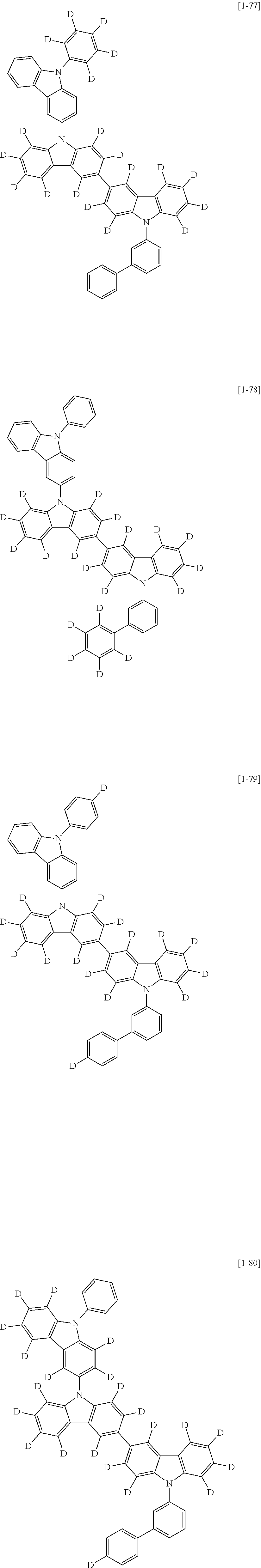

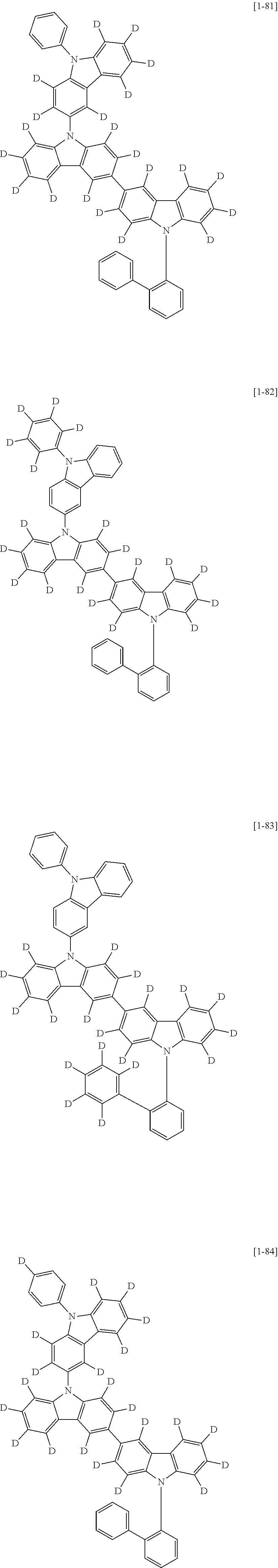

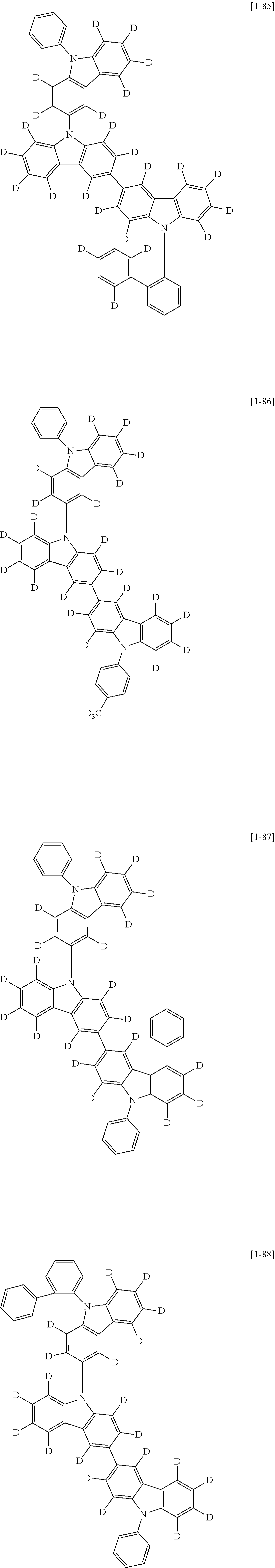

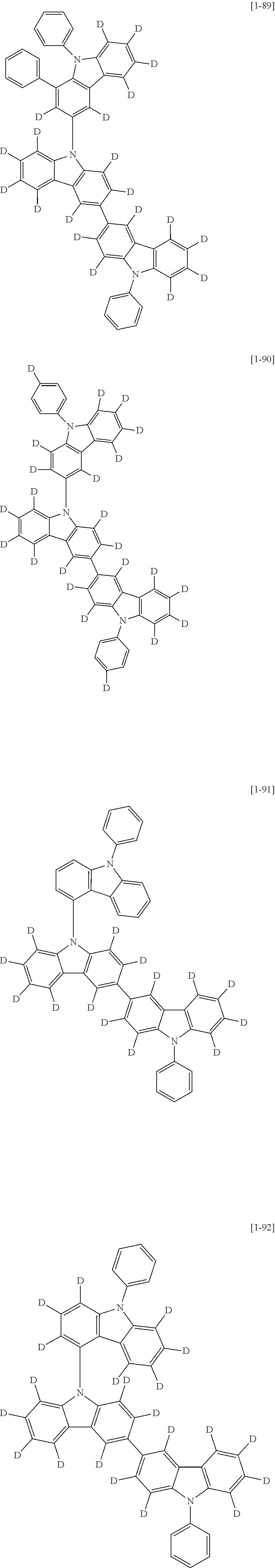

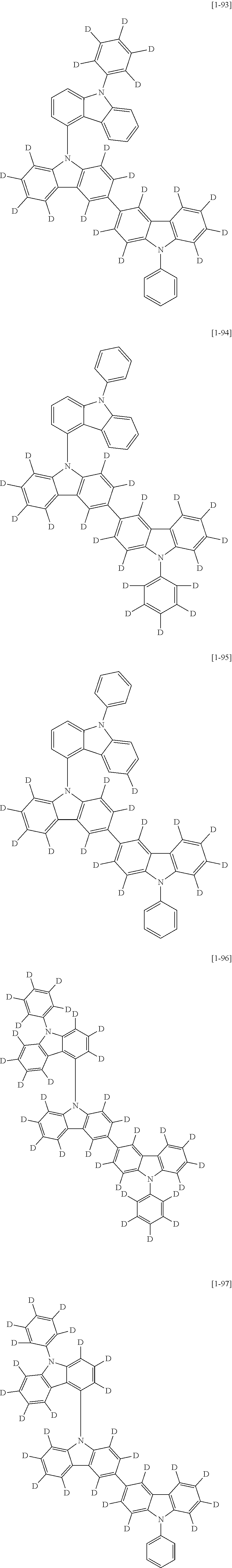

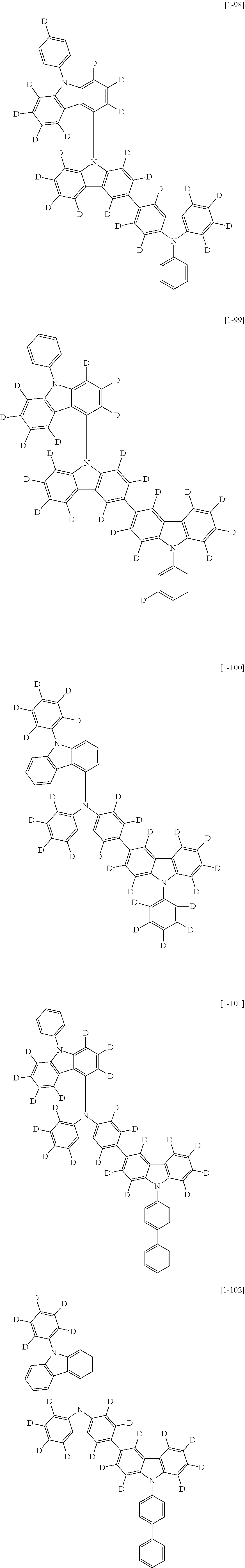

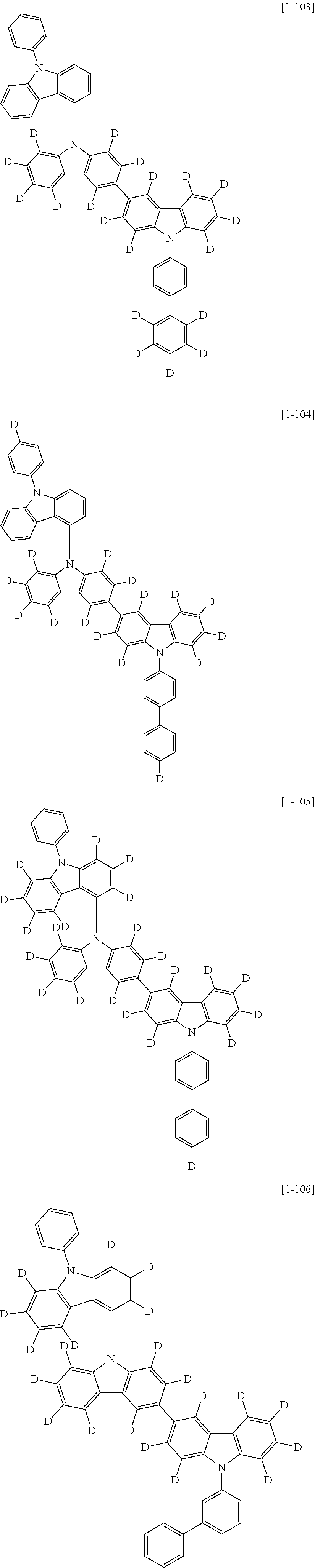

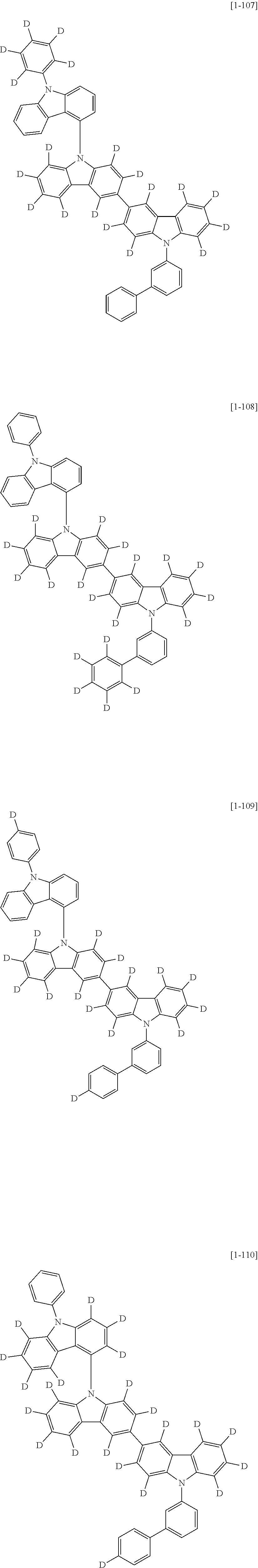

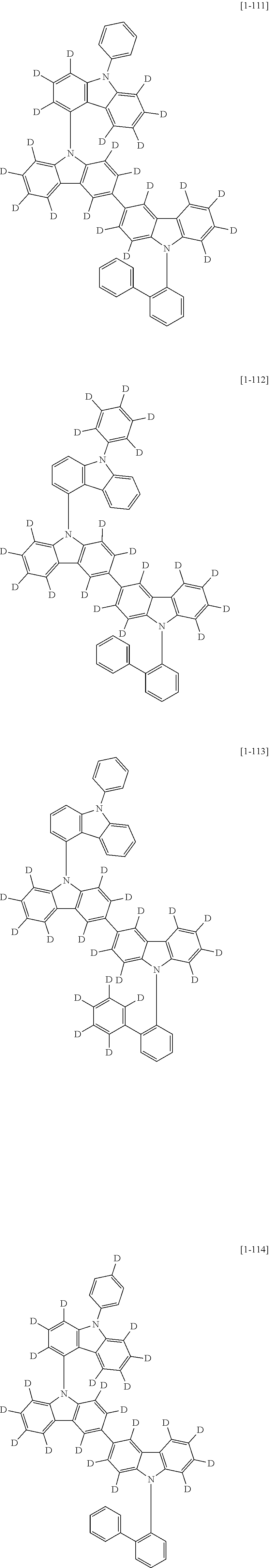

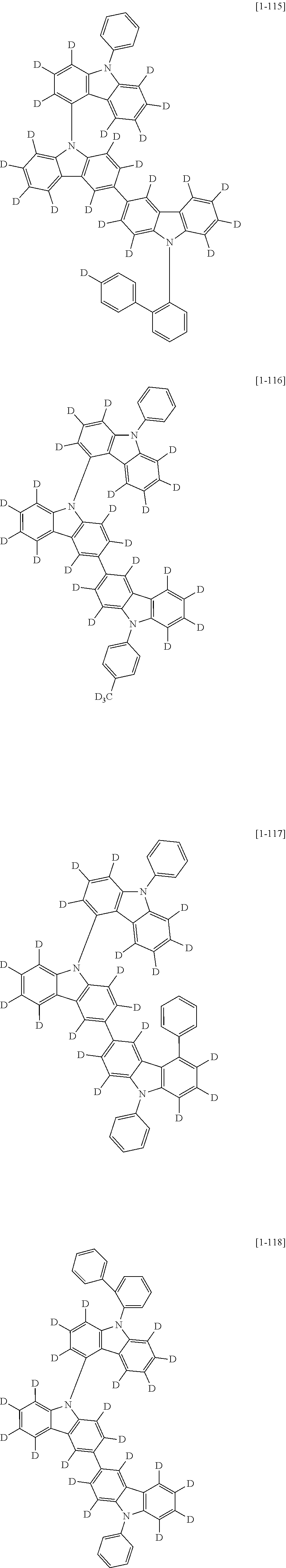

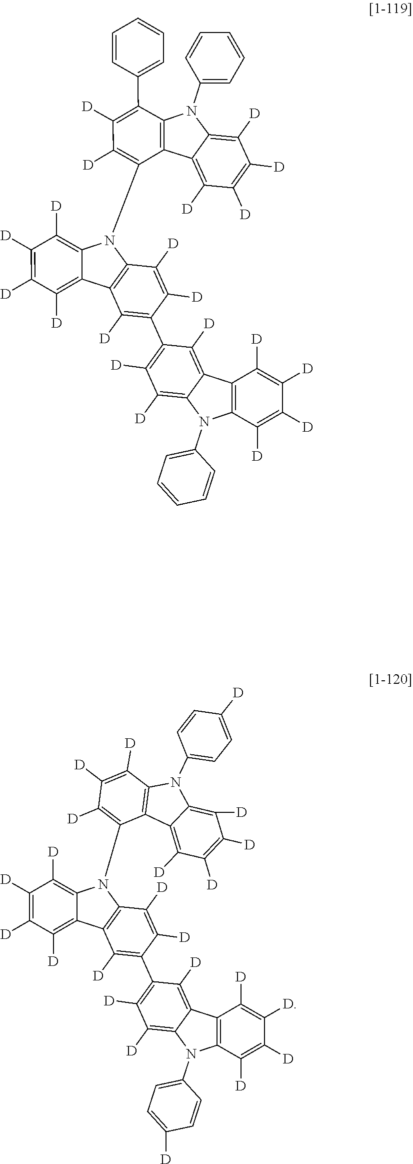

9. A composition for an organic optoelectronic device, the composition comprising:
a first compound; and
a second compound,
wherein:
the first compound is the compound for an organic optoelectronic device as claimed in claim 1, and
the second compound is a compound represented by Chemical Formula 2: 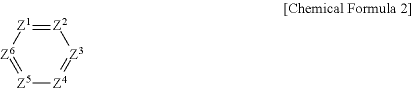
in Chemical Formula 2,
Z¹ is N or C-L³-R¹⁹,
Z² is N or C-L⁴-R²⁰,
Z³ is N or C-L⁵-R²¹,
Z⁴ is N or C-L⁶-R²²,
Z⁵ is N or C-L⁷-R²³,
Z⁶ is N or C-L⁸-R²⁴,
provided that at least two of Z¹ to Z⁶ are N,
L³ to L⁸ are each independently a single bond, a substituted or unsubstituted C6 to C20 arylene group, a substituted or unsubstituted C2 to C20 heterocyclic group, or a combination thereof,
R¹⁹ to R²⁴ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof,
R¹⁹ to R²⁴ are each separately present or adjacent ones thereof are linked to each other to provide a substituted or unsubstituted aliphatic monocyclic ring, a substituted or unsubstituted aliphatic polycyclic ring, a substituted or unsubstituted aromatic monocyclic ring, a substituted or unsubstituted to form an aromatic polycyclic ring, a substituted or unsubstituted heteroaromatic monocyclic ring, or a substituted or unsubstituted heteroaromatic polycyclic ring, and
when R¹⁹ to R²⁴ are each separately present, at least one of R¹⁹ to R²⁴ is a substituted or unsubstituted C10 to C30 aryl group or a substituted or unsubstituted C2 to C30 heterocyclic group.

10. The composition for an organic optoelectronic device as claimed in claim 9, wherein:
the second compound is represented by Chemical Formula 2-IB: 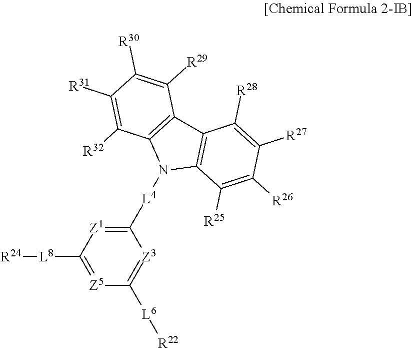
in Chemical Formula 2-IB,
Z¹, Z³, and Z⁵ are each independently N or CH, provided that at least two of Z¹, Z³, and Z⁵ are N,
L⁴, L⁶, and L⁸ are each independently a single bond, a substituted or unsubstituted C6 to C20 arylene group, a substituted or unsubstituted C2 to C20 heterocyclic group, or a combination thereof,
R²² and R²⁴ are each independently a substituted or unsubstituted C6 to C30 aryl group, a substituted or unsubstituted C2 to C30 heterocyclic group, or a combination thereof,
R²⁵ to R³² are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group, or a combination thereof, and
R²⁵ to R³² are each separately present or adjacent ones thereof are linked to each other to provide a substituted or unsubstituted aliphatic monocyclic ring, a substituted or unsubstituted aliphatic polycyclic ring, a substituted or unsubstituted aromatic monocyclic ring, a substituted or unsubstituted to form an aromatic polycyclic ring, a substituted or unsubstituted heteroaromatic monocyclic ring, or a substituted or unsubstituted heteroaromatic polycyclic ring.

11. The composition for an organic optoelectronic device as claimed in claim 10, wherein:
the second compound is represented by one of Chemical Formula 2-IB-1 to Chemical Formula 2-IB-6: 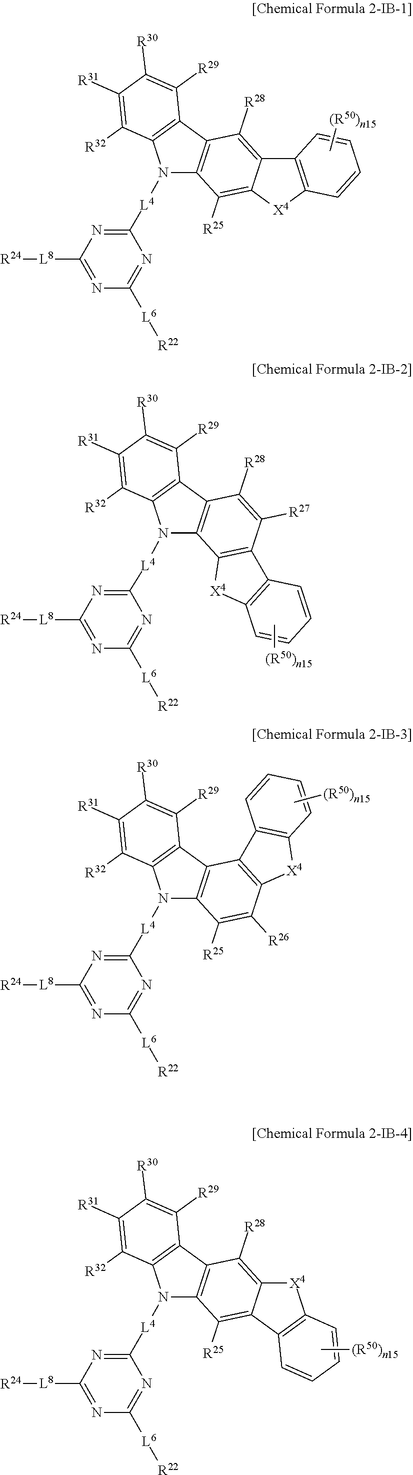 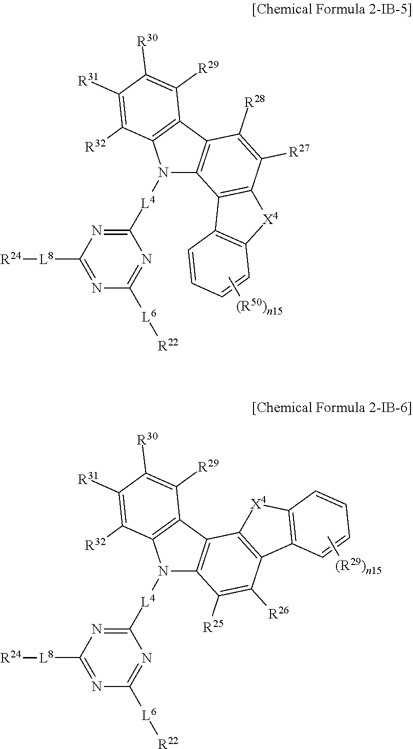
in Chemical Formula 2-IB-1 to Chemical Formula 2-IB-6,
X⁴ is O, S, CR^{j}R^{k}, or N-L¹⁴-Ar³,
n15 is an integer of 1 to 4,
L⁴, L⁶, L⁸, and L¹⁴ are each independently a single bond, a substituted or unsubstituted C6 to C20 arylene group, a substituted or unsubstituted C2 to C20 heterocyclic group, or a combination thereof,
R^{j} and R^{k} are each independently a substituted or unsubstituted C1 to C20 alkyl group, a substituted or unsubstituted C6 to C30 aryl group, or a combination thereof,
Ar³, R²², and R²⁴ are each independently a substituted or unsubstituted C6 to C30 aryl group, a substituted or unsubstituted C2 to C30 heterocyclic group, or a combination thereof, and
R²⁵ to R³² and R⁵⁰ are each independently hydrogen, deuterium, a substituted or unsubstituted C1 to C30 alkyl group, a substituted or unsubstituted C6 to C30 aryl group, a substituted or unsubstituted C2 to C30 heterocyclic group, a substituted or unsubstituted silyl group, a substituted or unsubstituted amine group, halogen, a cyano group or a combination thereof.

12. An organic optoelectronic device, comprising:
an anode and a cathode facing each other,
at least one organic layer between the anode and the cathode,
wherein the at least one organic layer includes the compound for an organic optoelectronic device as claimed in claim 1.

13. The organic optoelectronic device as claimed in claim 12, wherein:
the at least one organic layer includes a light emitting layer, a hole transport layer between the anode and the cathode, and a hole transport auxiliary layer between the light emitting layer and the hole transport layer, and
the hole transport auxiliary layer includes the compound for an organic optoelectronic device.

14. A display device comprising the organic optoelectronic device as claimed in claim 12.

15. An organic optoelectronic device, comprising:
an anode and a cathode facing each other,
at least one organic layer between the anode and the cathode,
wherein the at least one organic layer includes the composition for an organic optoelectronic device as claimed in claim 9.

16. The organic optoelectronic device as claimed in claim 15, wherein:
the at least one organic layer includes a light emitting layer, and
the light emitting layer includes the composition for an organic optoelectronic device.

17. A display device comprising the organic optoelectronic device as claimed in claim 15.
